(19) United States
(12) Patent Application Publication
(10) Pub. No.: US 2020/0227658 A1
(43) Pub. Date: Jul. 16, 2020

(54) ORGANIC COMPOUND AND ORGANIC ELECTROLUMINESCENCE DEVICE USING THE SAME

(71) Applicants: FENG-WEN YEN, Taipei (TW); TSUN-YUAN HUANG, CHIAYI (TW)

(72) Inventors: FENG-WEN YEN, Taipei (TW); TSUN-YUAN HUANG, CHIAYI (TW)

(73) Assignee: LUMINESCENCE TECHNOLOGY CORPORATION, HSINCHU (TW)

(21) Appl. No.: 16/248,743

(22) Filed: Jan. 15, 2019

(51) Int. Cl. H01L 51/00; C07D 307/92; C09K 11/06; C07D 405/10; C07D 409/10; C07F 7/08; C07D 307/77; C09K 11/02

(52) U.S. Cl. CPC H01L 51/0073; C07D 307/92; C09K 11/06; C07D 405/10; H01L 51/0072; C07D 409/10; H01L 51/5016; H01L 51/0067; H01L 51/0094; C07F 7/0807; C07D 307/77; C09K 11/025; H01L 51/0074

(57) ABSTRACT

The present invention discloses an organic compound represented by the following formula (1) and an organic electroluminescence device using the organic compound as the phosphorescent host material, the fluorescent host material, or the fluorescent dopant material. The organic compound may lower a driving voltage or power consumption, or may increase a current efficiency or half-life of the organic electroluminescence device.

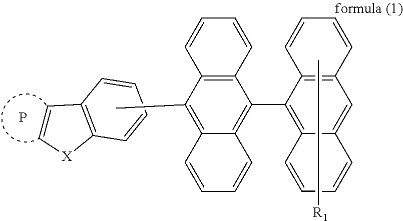

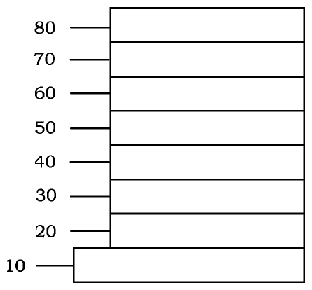

The same definition as described in the present invention.

### FIELD OF INVENTION

The present invention relates to a novel organic compound and, more particularly, to an organic electroluminescence device using the organic compound.

### BACKGROUND OF THE INVENTION

An organic electroluminescence (organic EL) device is an organic light-emitting diode (OLED) in which the light emitting layer is a film made from organic compounds, which emits light in response to the electric current. The light emitting layer containing the organic compound is sandwiched between two electrodes. The organic EL device is applied to flat panel displays due to its high illumination, low weight, ultra-thin profile, self-illumination without back light, low power consumption, wide viewing angle, high contrast, simple fabrication methods and rapid response time.

Typically, the organic EL device is composed of organic material layers sandwiched between two electrodes. The organic material layers include, e.g., hole injection layer(HIL), hole transporting layer (HTL), emitting layer (EML), electron transporting layer (ETL), and electron injection layer (EIL). The basic mechanism of organic EL involves the injection, transport, and recombination of carriers as well as exciton formation for emitting light. When an external voltage is applied across the organic EL device, electrons and holes are injected from the cathode and the anode, respectively. Electrons will be injected from the cathode into a LUMO (lowest unoccupied molecular orbital) and holes will be injected from the anode into a HOMO (highest occupied molecular orbital). Subsequently, the electrons recombine with holes in the light emitting layer to form excitons, which then deactivate to emit light. When luminescent molecules absorb energy to achieve an excited state, the exciton may either be in a singlet state or a triplet state, depending on how the spins of the electrons and holes have been combined. It is well known that the excitons formed under electrical excitation typically include 25% singlet excitons and 75% triplet excitons. In the fluorescence materials, however, the electrically generated energy in the 75% triplet excitons will be dissipated as heat for decay from the triplet state is spin forbidden. Therefore, a fluorescent electroluminescence device has only 25% internal quantum efficiency, which leads to the theoretically highest external quantum efficiency (EQE) of only 5% due to only -20% of the light out-coupling efficiency of the device. In contrast to fluorescent electroluminescence devices, phosphorescent organic EL devices make use of spin-orbit interactions to facilitate intersystem crossing between singlet and triplet states, thus obtaining emission from both singlet and triplet states and the internal quantum efficiency of electroluminescence devices from 25% to 100%.

However, there is still a need for improvement in the case of use of those organic materials in an organic EL device of some prior art displays, for example, in relation to the half-life time, current efficiency or driving voltage of the organic EL device.

### SUMMARY OF THE INVENTION

Accordingly, an object of the invention is to provide an organic compound and an organic EL device using the same, which can exhibit improved luminance, current efficiency, or half-life time.

Another object of the invention is to provide an organic compound and an organic EL device using the same, which may lower a driving voltage or increasing a current efficiency or half-life time of the organic EL device.

Still another object of the present invention is to provide an organic compound, which can be used as a phosphorescent host material, a fluorescent host material, or a fluorescent dopant material in the emitting layer, and/or an electron transporting material in an organic EL device to improve the power consumption, luminance, current efficiency, or life time.

According to the present invention, one organic compound which may be used in an organic EL device is disclosed. The organic compound may be represented by the following formula (1):

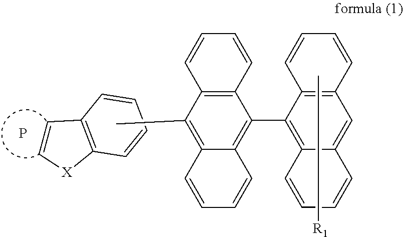

wherein X may be a divalent bridge selected from the group consisting of O, S, and SiR₂R₃. P may represent a substituted or unsubstituted fused ring hydrocarbons unit having two, three or four rings. The fused ring hydrocarbons unit may be, for example, a polycyclic aromatic hydrocarbons (PAHs) unit. R₁ to R₃ may independently represent a hydrogen atom, a methyl group, a halide (e.g., fluoride), a substituted or unsubstituted alkyl group having 1 to 30 (e.g., 1, 3, 6, 8 or 12) carbon atoms, a substituted or unsubstituted alkoxy group having 1 to 30 (e.g., 1, 6 or 8) carbon atoms, a substituted or unsubstituted aryl group having 6 to 30 carbon atoms, a substituted or unsubstituted aralkyl group having 7 to 30 (e.g., 8 or 9) carbon atoms, or a substituted or unsubstituted heteroaryl group having 3 to 30 carbon atoms. The heteroaryl group may comprise, for example, a heteroaromatic PAHs unit having two, three, four, five or six rings. The heteroaromatic PAHs may contain an oxygen atom, a sulfur atom or one, two or three N atoms.

The substituted aryl group may be an aryl group substituted by an alkoxy group or by a methyl or ethyl substituted heteroaromatic PAHs unit having two rings. The two-rings heteroaromatic PAHs may contain two N atoms.

R₁ may also represent a phenyl group, a naphthyl group, a dibenzofuranyl group, a benzo[b]naphtho[2,3-d]furanyl group, an isopropyl-benzo[b]naphtho[2,1-d]furanyl group, a carbazole group, a N-phenylcarbazole group, a trifluoromethyl group, a cumene (isopropylbenzene) group, a phenyl-phenylpyrimidine group, a biphenyl-phenylpyrimidine group, a diphenyl-triazine group or a 4,6-diphenyl-1,3,5-triazine group.

P may represent a polycyclic aromatic hydrocarbons (PAHs) unit having two, three, four or five rings. P may comprise, for example, a naphthyl group, an anthracenyl group, a phenanthrenyl group, a triphenylenyl group or a pyrenyl group. Each of the groups may be substituted by, for example, an isopropyl group, an isobutyl group or a hexyl group.

The present invention may further disclose an organic compound which may be used in an organic EL device is disclosed.

The present invention may further disclose another organic compound which may be used in an organic EL device. The organic compound may be represented by the following formula (2):

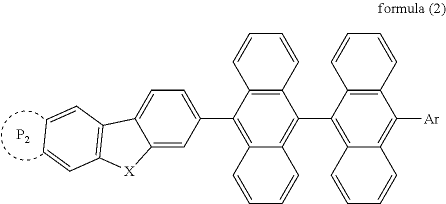

Wherein Ar may represent a non-heteroaryl group having 3 to 30 carbon atoms. The non-heteroaryl group may be a phenyl group, a naphthyl group or a phenathrenyl group. P₂ may represent a substituted or unsubstituted fused ring hydrocarbons unit having one, two or three rings. The fused ring hydrocarbons unit may be, for example, a PAHs unit. The PAHs unit may be a phenyl group, a naphthyl group or a phenathrenyl group.

An organic EL device of the present invention may comprise an organic compound of formula (2) as a host material to emit a blue light, thereby increasing a current efficiency to about 7.1-7.3 cd/A, lowering a driving voltage to about 3.1-3.2 V, or increasing a half-life time to about 300-340 hours.

An organic EL device of the present invention comprises an organic compound of formula (1) as a dopant material to collocate with, for example, a host material H1 to emit a green light, thereby lowering a driving voltage to about 5.2-5.4 V, increasing a current efficiency to about 51.3-52.4 cd/A, or increasing a half-life time to about 480-500 hours. The organic EL device of may comprise an organic compound of formula (1) as a dopant material to collocate with a host material H2 to emit a green light, thereby lowering a driving voltage to about 5.1-5.3 V, increasing a current efficiency to about 52.1-53.9 cd/A, or increasing a half-life time to about 500-520 hours.

The organic EL device of may comprise an organic compound of formula (1) as a dopant material to collocate with a host material H1 to emit a blue light, thereby lowering a driving voltage to about 3.4-4.4 V, increasing a current efficiency to about 5.0-6.5 cd/A, or increasing a half-life time to about 130-280 hours.

Alternatively, an organic EL device of may comprise an organic compound of formula (1) as a dopant material to collocate with a host material H1 to emit a blue light, thereby lowering a driving voltage to about 3.4-4.4 V, increasing a current efficiency to about 5.0-6.5 cd/A, or increasing a half-life time to about 130-280 hours. The organic EL device of may comprise an organic compound of formula (1) as a dopant material to collocate with a host material H2 to emit a blue light, thereby lowering a driving voltage to about 3.3-4.2 V, increasing a current efficiency to about 5.1-6.9 cd/A, or increasing a half-life time to about 190-340 hours.

An organic EL device of the present invention may comprise an organic compound of formula (1) as a host material to collocate with a dopant material D1 to emit a blue light, thereby lowering a driving voltage to about 3.1-4.2 V, increasing a current efficiency to about 4.7-7.3 cd/A, or increasing a half-life time to about 160-340 hours.

### BRIEF DESCRIPTION OF THE DRAWINGS

The FIGURE is a schematic view showing an organic EL device according to an embodiment of the present invention.

### DESCRIPTION OF THE PREFERRED EMBODIMENTS

What probed into the invention is the organic compound and organic EL device using the organic compound. Detailed descriptions of the production, structure and elements will be provided as follows such that the invention can be fully understood. Obviously, the application of the invention is not confined to specific details familiar to those skilled in the art. On the other hand, the common elements and procedures that are known to everyone are not described in details to avoid unnecessary limits of the invention. Some preferred embodiments of the present invention will now be described in greater detail as follows. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In one embodiment of the present invention, an organic compound which can be used as the phosphorescent host material, the fluorescent host material, or the fluorescent dopant material of the light emitting layer of a light emitting layer for an organic EL device is disclosed. The organic compound is represented by the following formula (1):

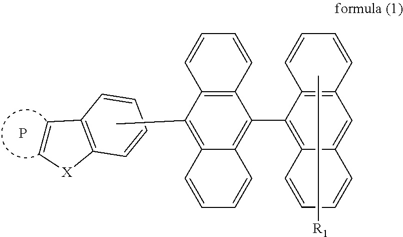

wherein X is a divalent bridge selected from the group consisting of O, S, and SiR₂R₃, P may represent a substituted or unsubstituted fused ring hydrocarbon unit having two, three or four rings. R₁ to R₃ may independently represent a hydrogen atom, a methyl group, a halide (e.g., fluoride), a substituted or unsubstituted alkyl group having 1 to 30 carbon atoms, a substituted or unsubstituted alkoxy group having 1 to 30 carbon atoms, a substituted or unsubstituted aryl group having 6 to 30 carbon atoms, a substituted or unsubstituted aralkyl group having 7 to 30 carbon atoms, or a substituted or unsubstituted heteroaryl group having 3 to 30 carbon atoms. In certain embodiments, the substituted alkyl group, the substituted alkoxy group, the substituted aryl group, the substituted aralkyl group, or the substituted heteroaryl group is substituted by a halide, an alkyl group, an alkoxy group, or an aryl group.

The organic compound may be one of the following compounds:

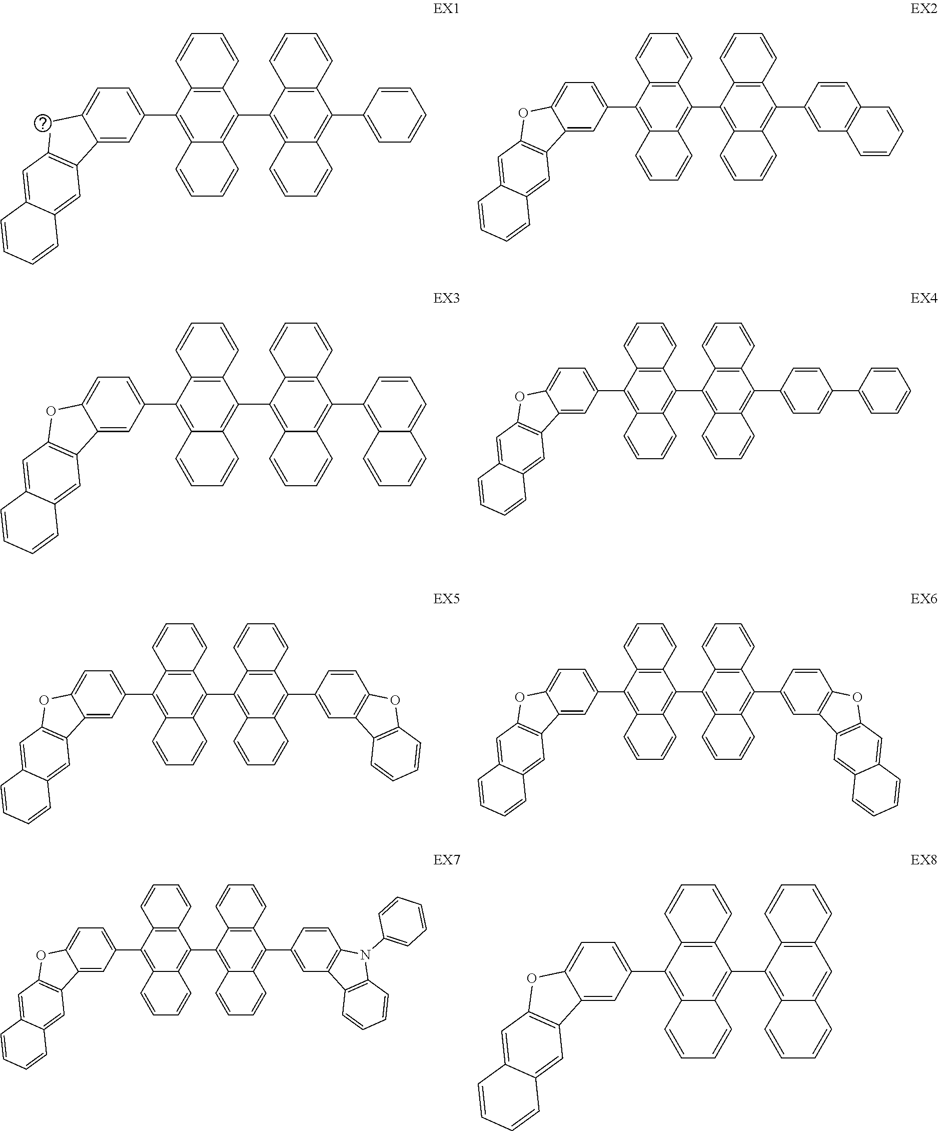

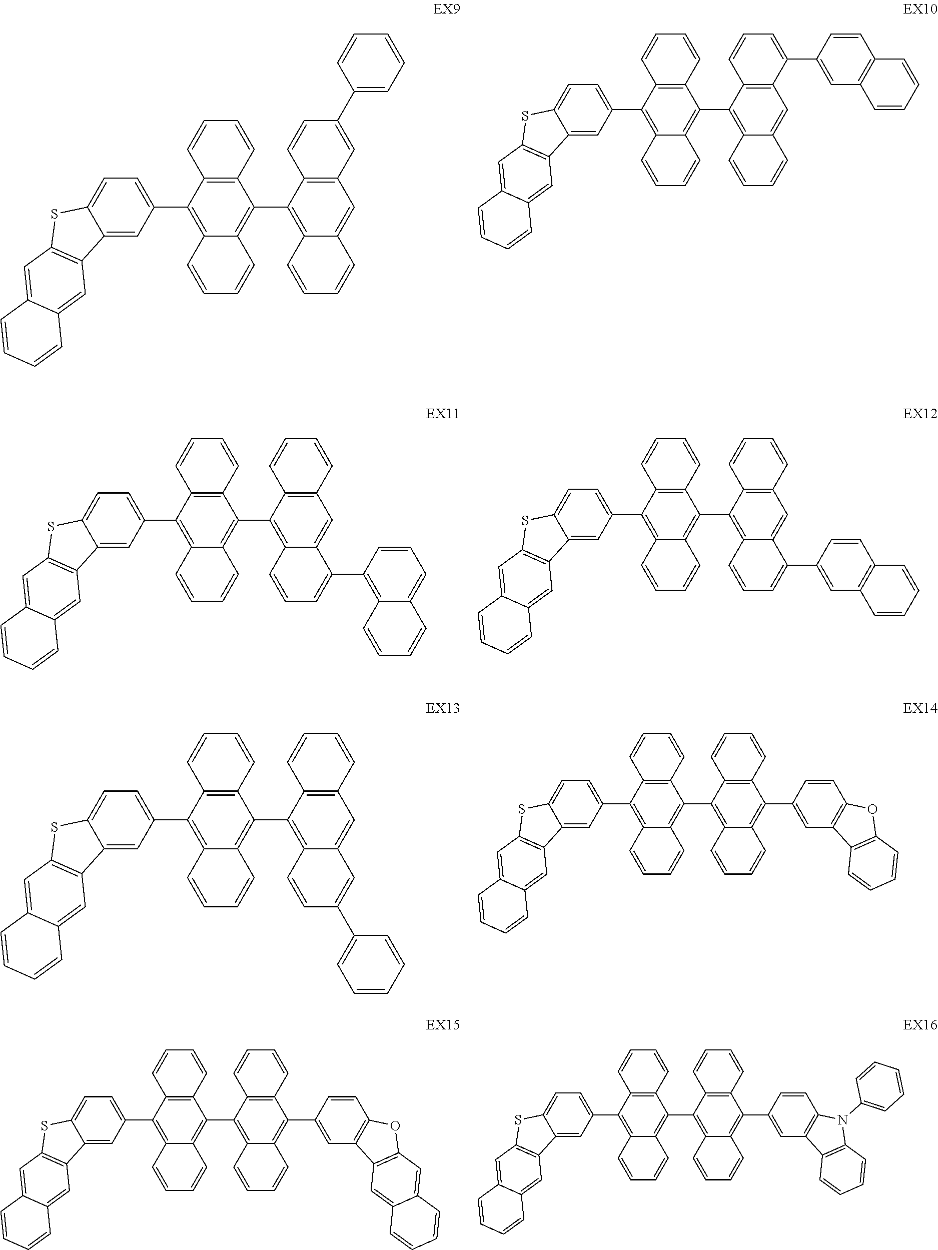

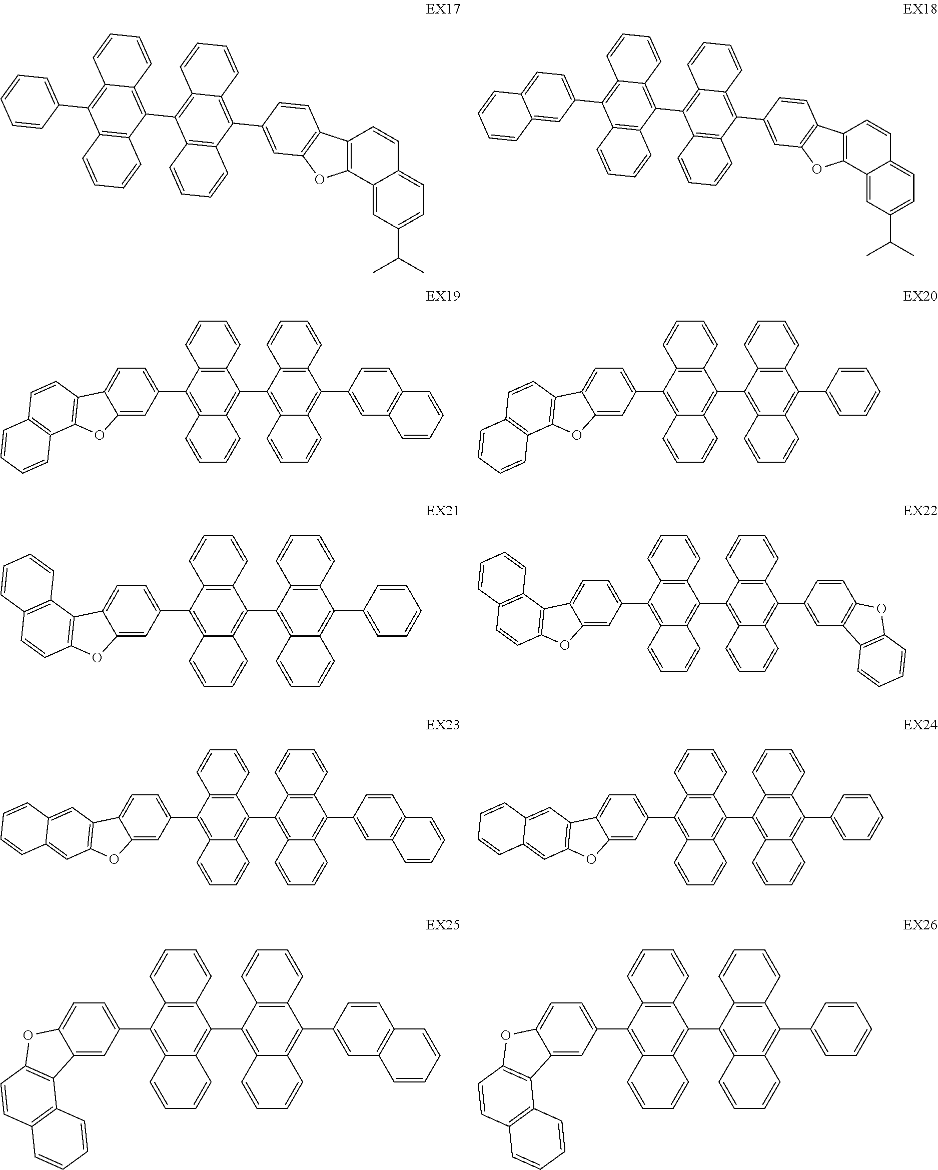

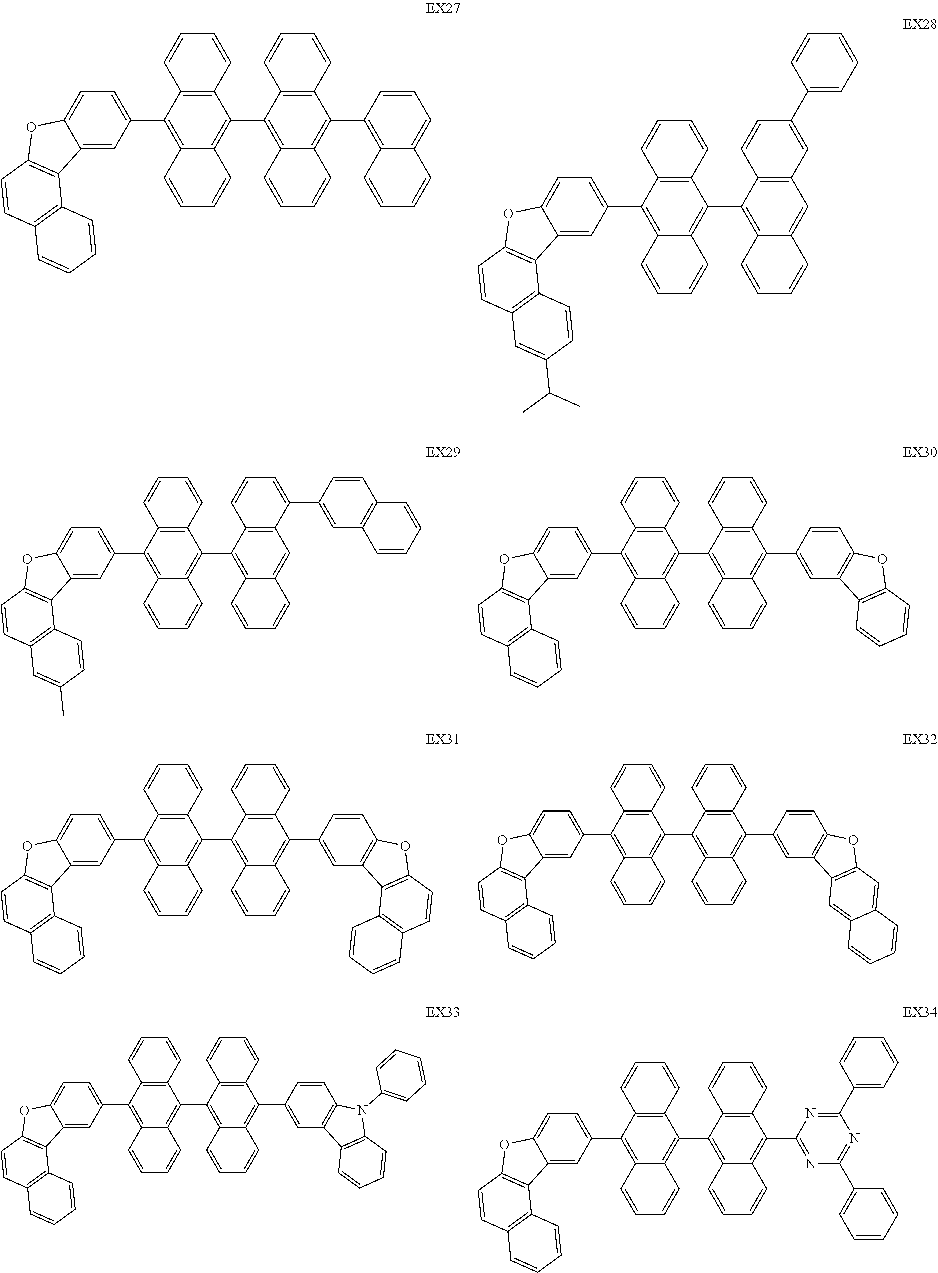

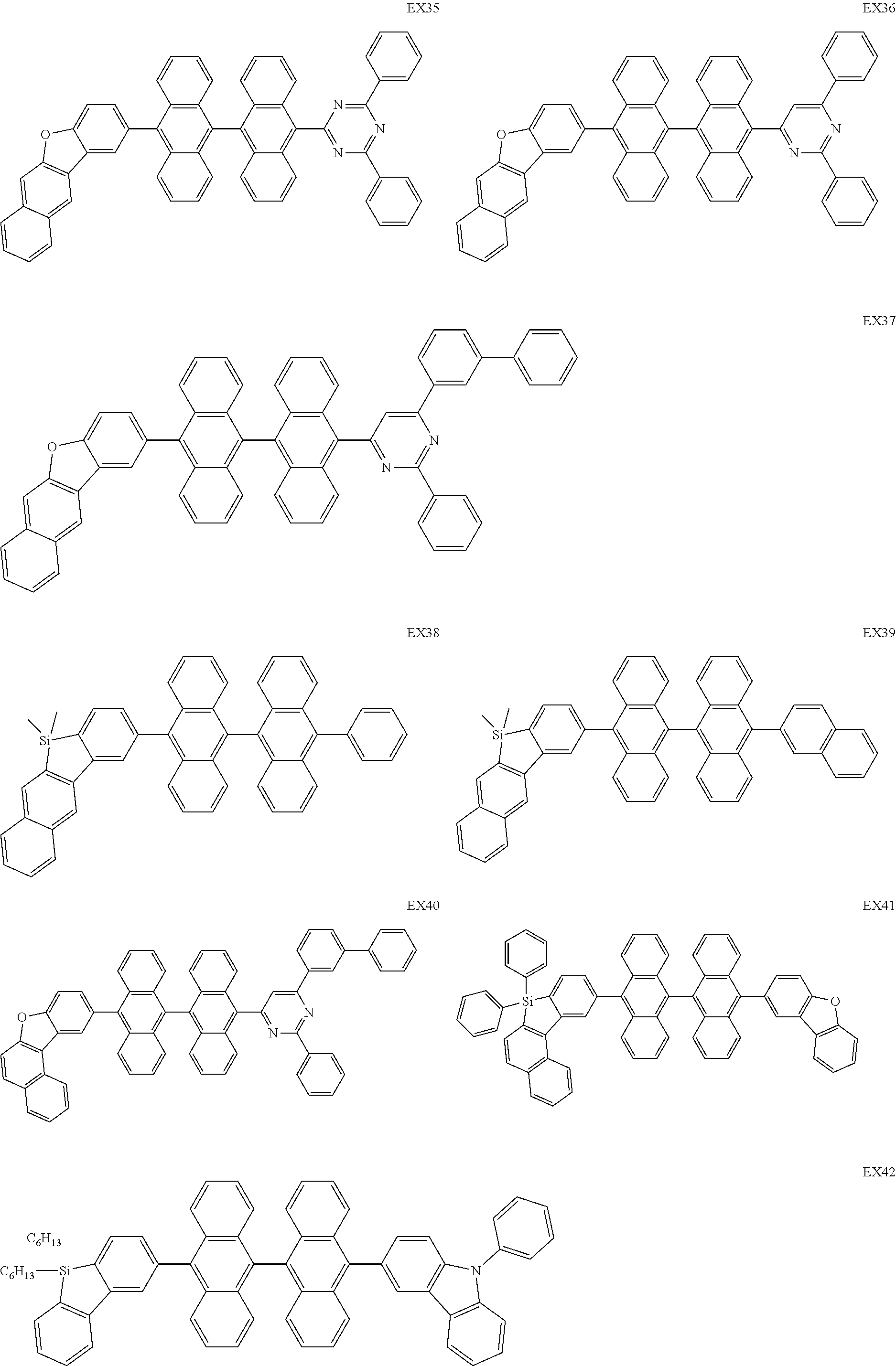

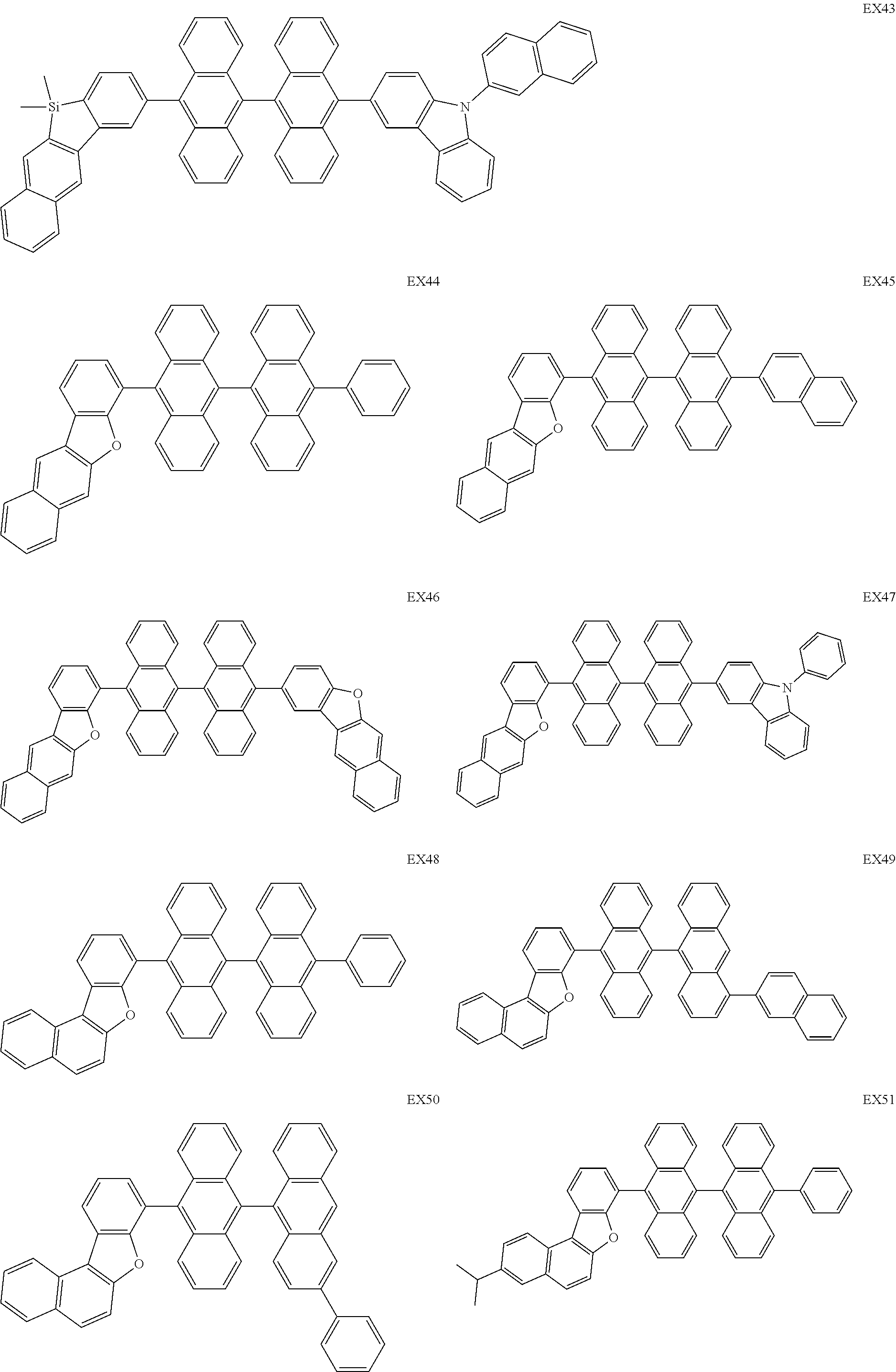

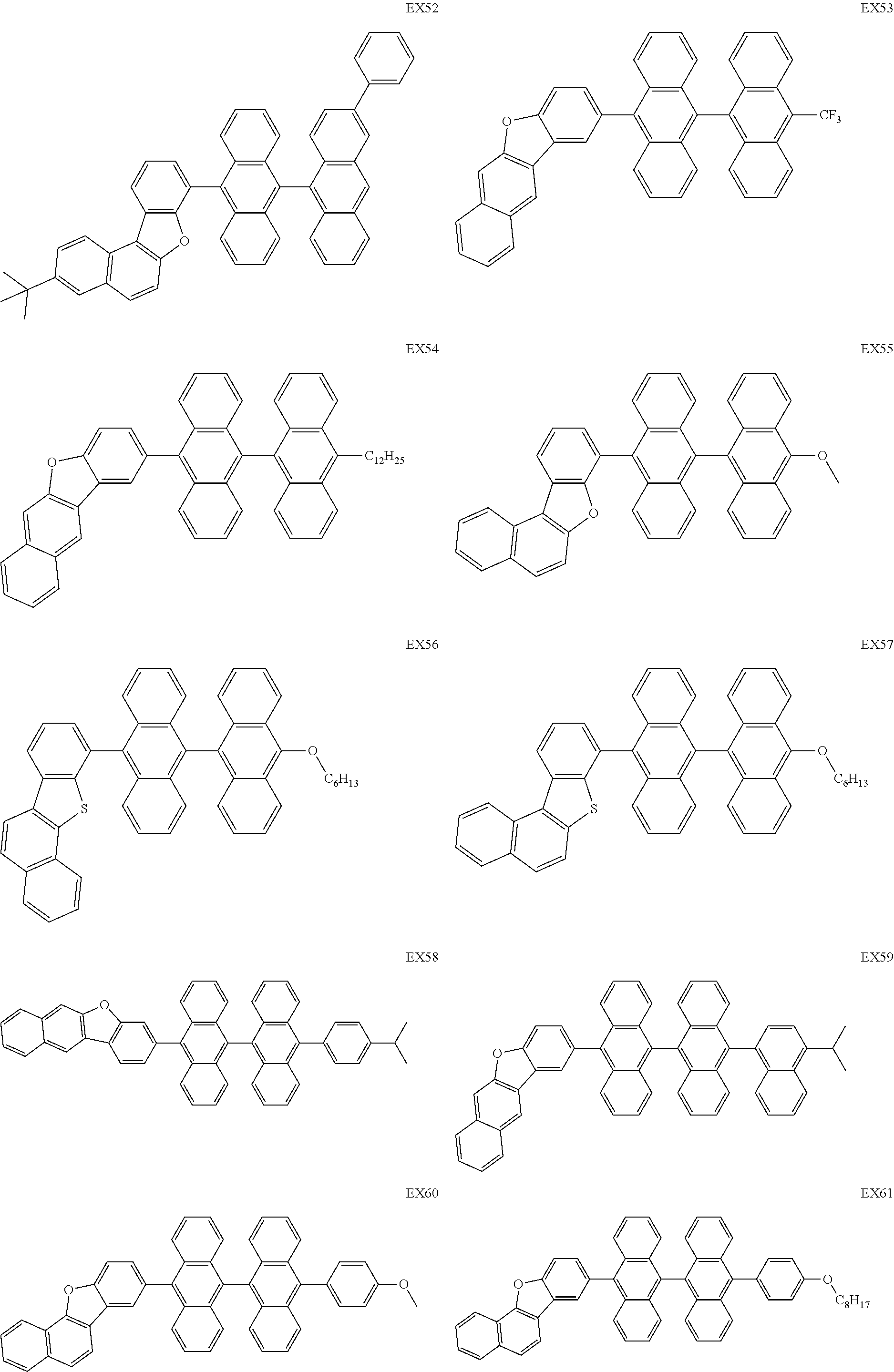

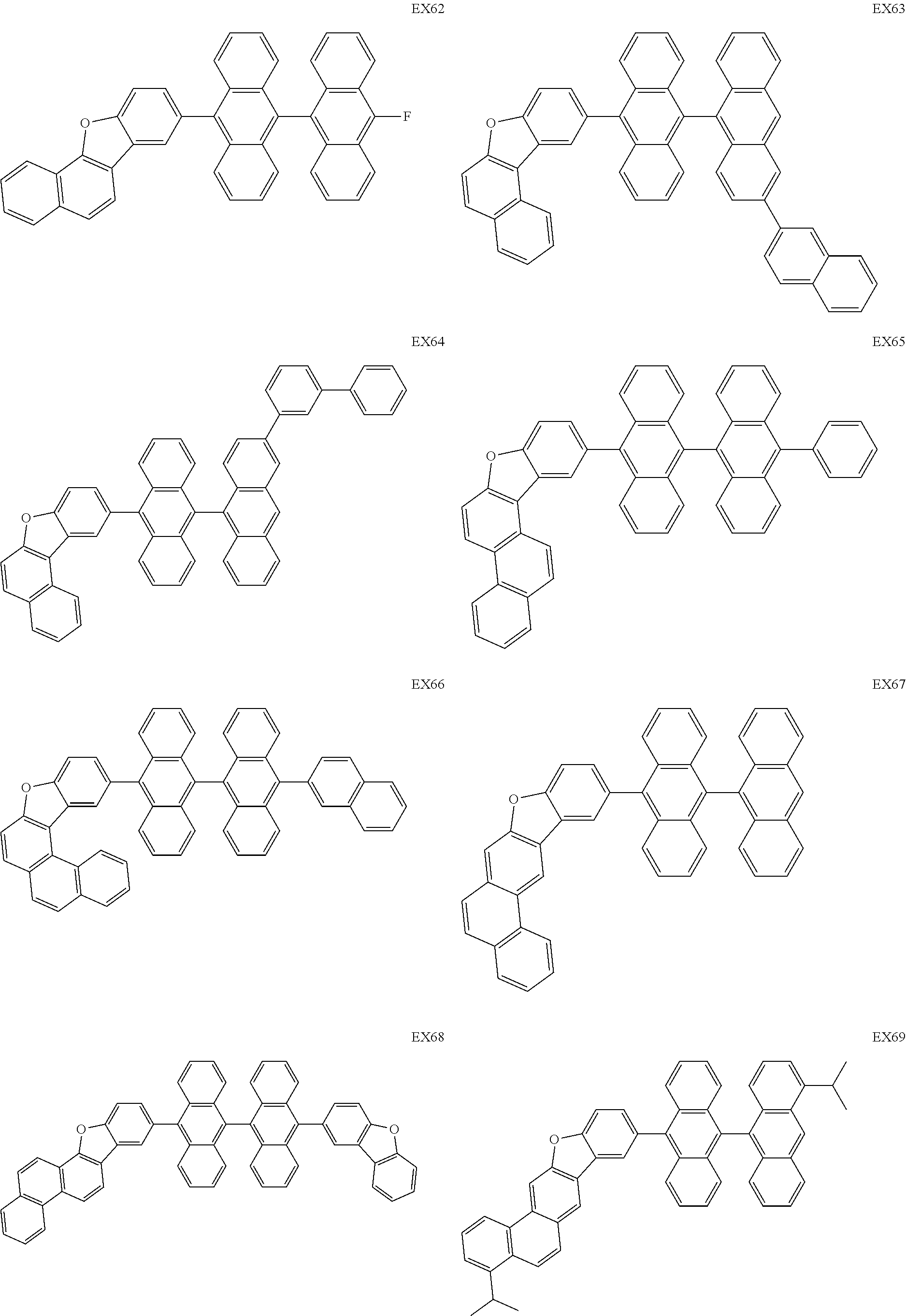

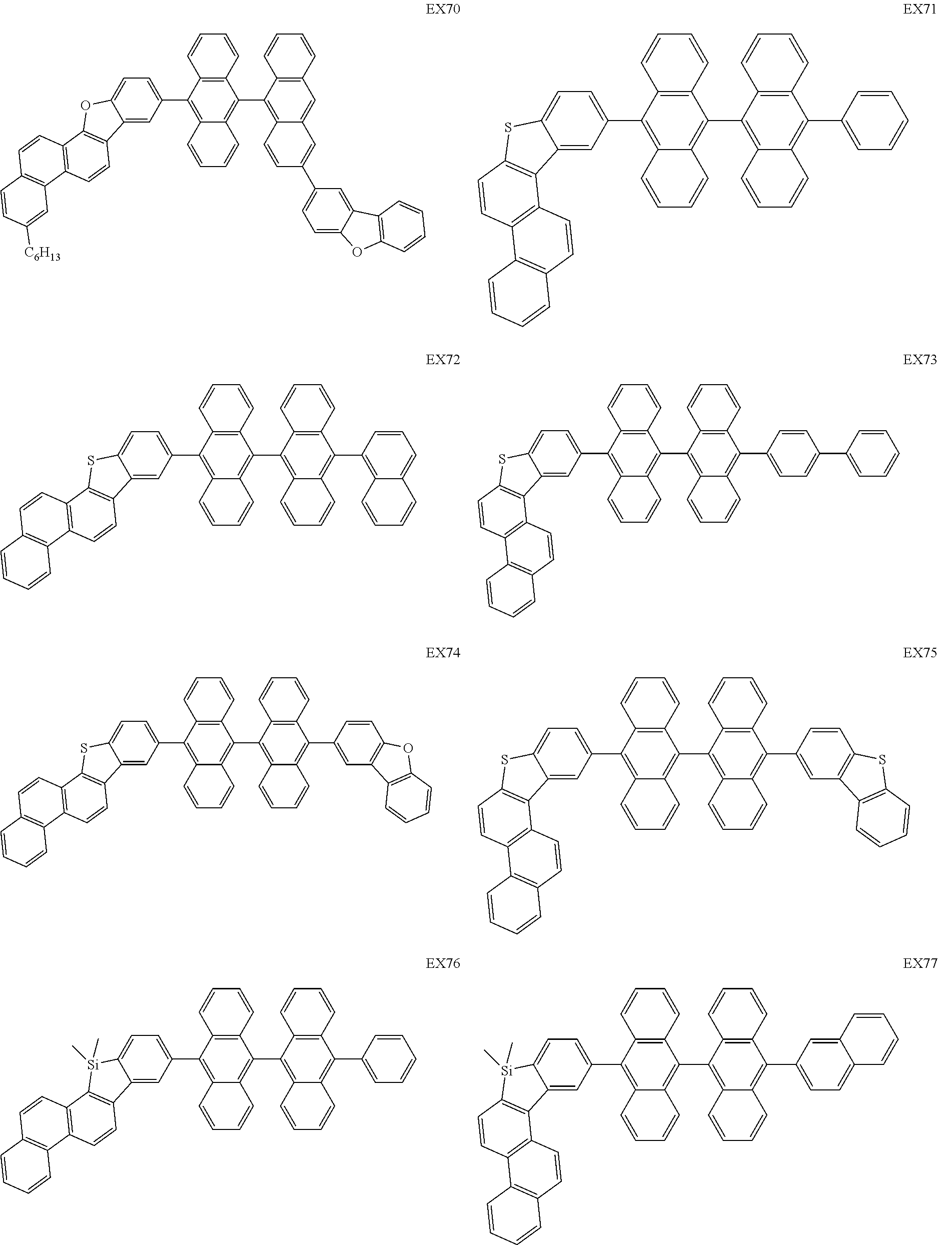

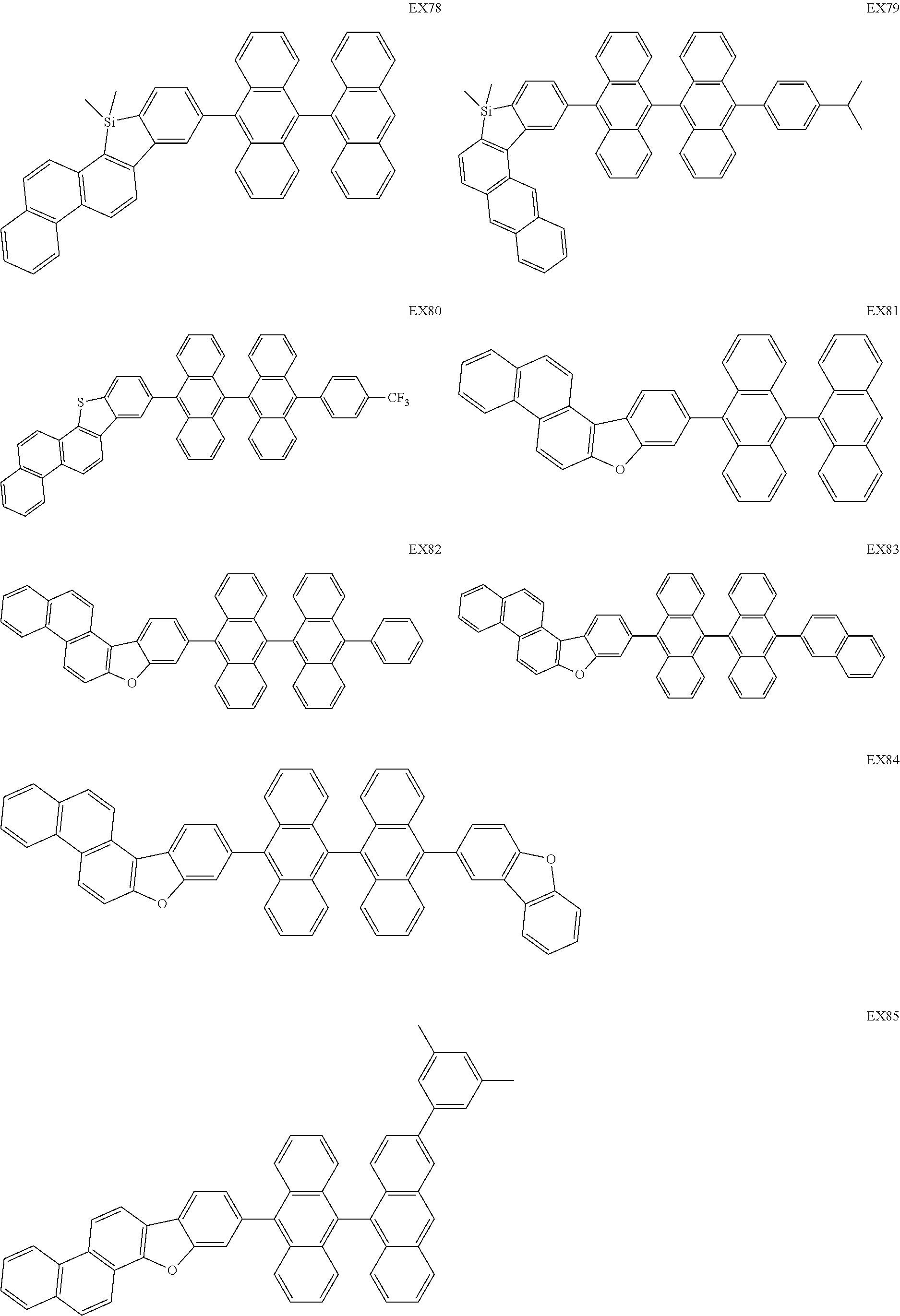

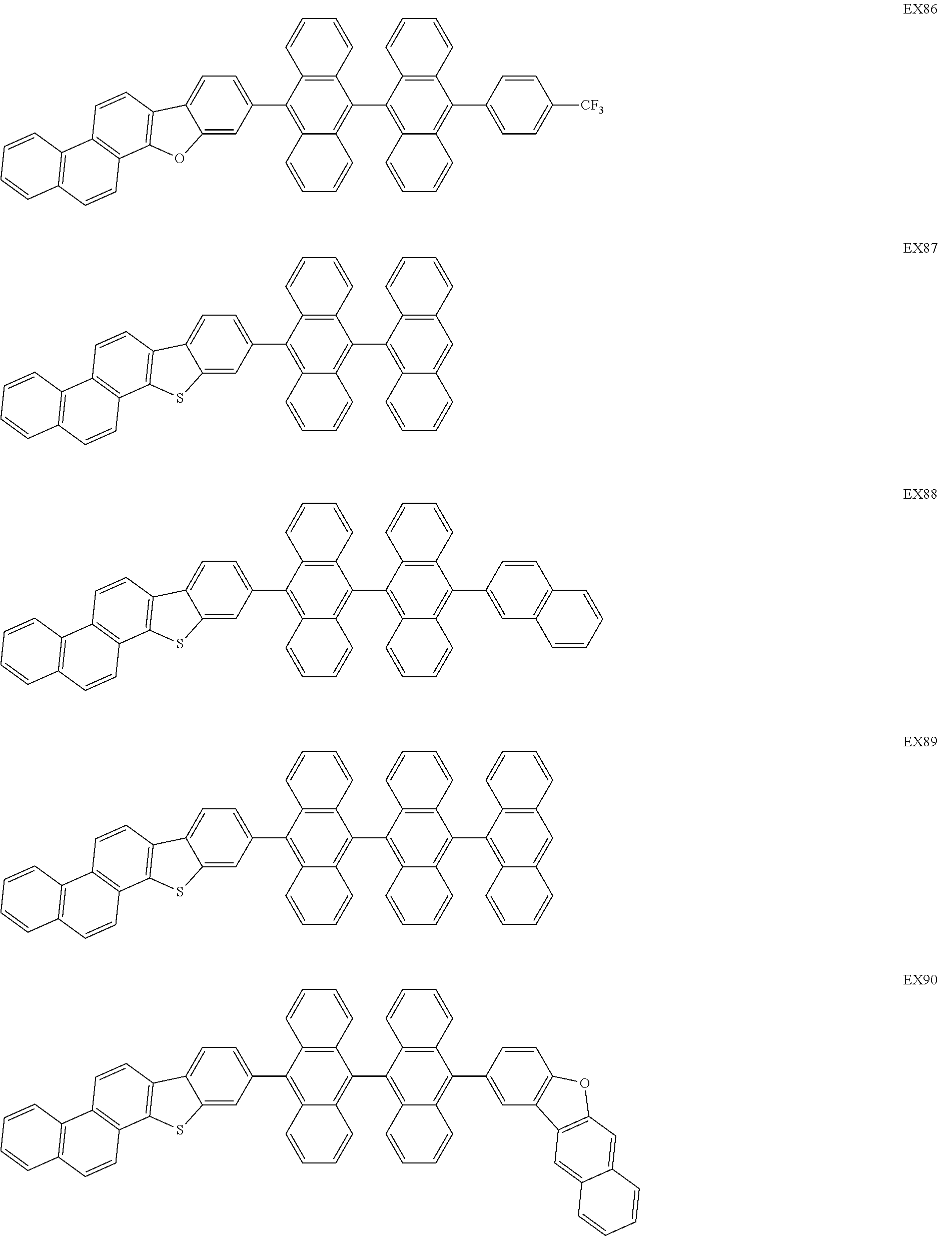 [SMILES: FC(F)(F)c1ccc(-c2c3ccccc3c(-c3c4ccccc4c(-c4ccc5c(c4)oc4c5ccc5c6ccccc6ccc54)c4ccccc34)c3ccccc23)cc1 ; c1ccc2c(-c3c4ccccc4c(-c4ccc5c(c4)sc4c5ccc5c6ccccc6ccc54)c4ccccc34)c3ccccc3cc2c1 ; c1ccc2cc(-c3c4ccccc4c(-c4c5ccccc5c(-c5ccc6c(c5)sc5c6ccc6c7ccccc7ccc65)c5ccccc45)c4ccccc34)ccc2c1 ; c1ccc2c(-c3c4ccccc4c(-c4c5ccccc5c(-c5ccc6c(c5)sc5c6ccc6c7ccccc7ccc65)c5ccccc45)c4ccccc34)c3ccccc3cc2c1 ; c1ccc2cc3c(cc2c1)oc1ccc(-c2c4ccccc4c(-c4c5ccccc5c(-c5ccc6c(c5)sc5c6ccc6c7ccccc7ccc65)c5ccccc45)c4ccccc24)cc13]

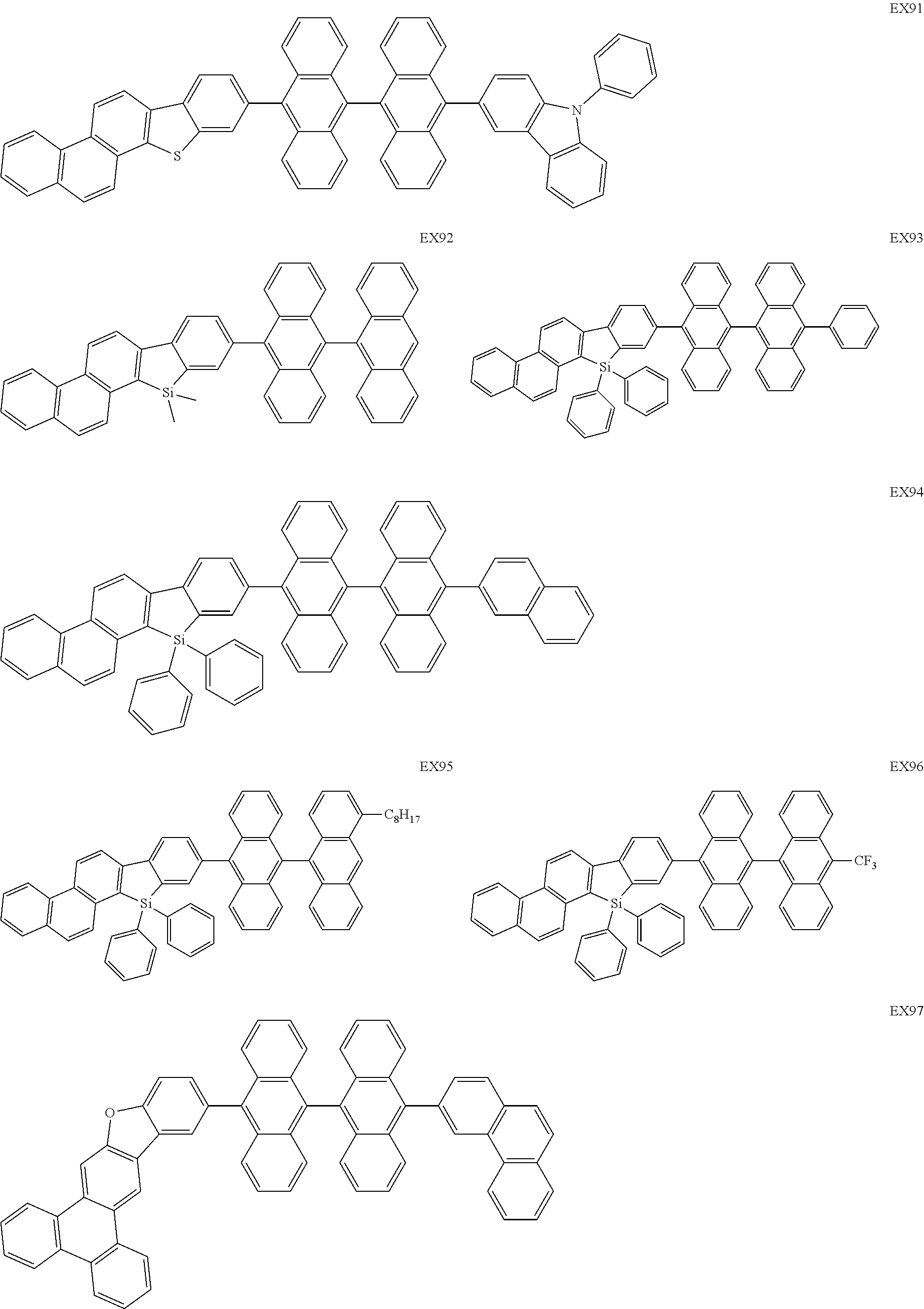

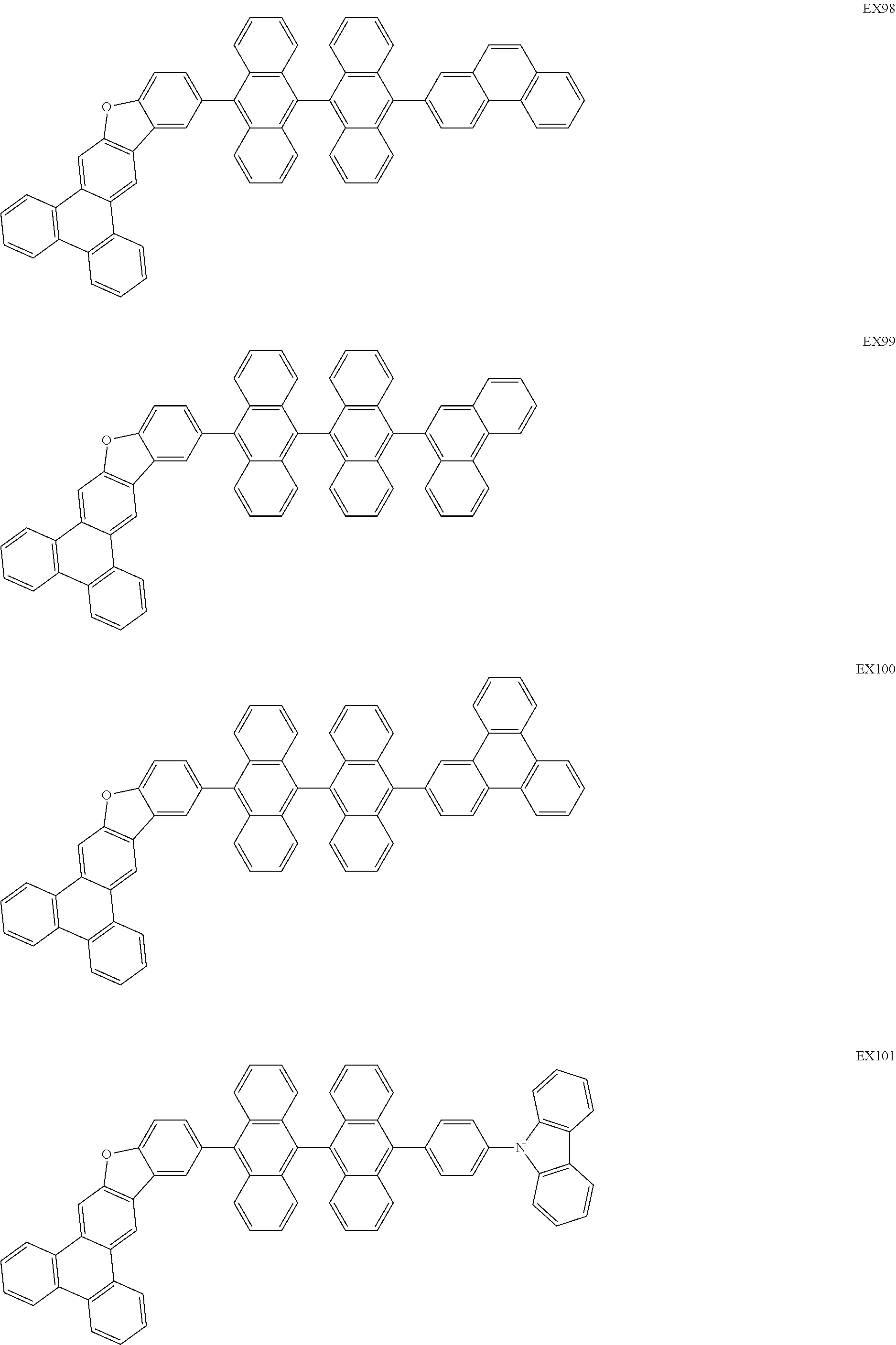 [SMILES: c1ccc2c(c1)ccc1cc(-c3c4ccccc4c(-c4c5ccccc5c(-c5ccc6oc7cc8c9ccccc9c9ccccc9c8cc7c6c5)c5ccccc45)c4ccccc34)ccc12 ; c1ccc2c(c1)cc(-c1c3ccccc3c(-c3c4ccccc4c(-c4ccc5oc6cc7c8ccccc8c8ccccc8c7cc6c5c4)c4ccccc34)c3ccccc13)c1ccccc12 ; c1ccc2c(-c3c4ccccc4c(-c4ccc5c6ccccc6c6ccccc6c5c4)c4ccccc34)c3ccccc3c(-c3ccc4oc5cc6c7ccccc7c7ccccc7c6cc5c4c3)c2c1 ; c1ccc2c(-c3c4ccccc4c(-c4ccc5oc6cc7c8ccccc8c8ccccc8c7cc6c5c4)c4ccccc34)c3ccccc3c(-c3ccc(-n4c5ccccc5c5ccccc54)cc3)c2c1]

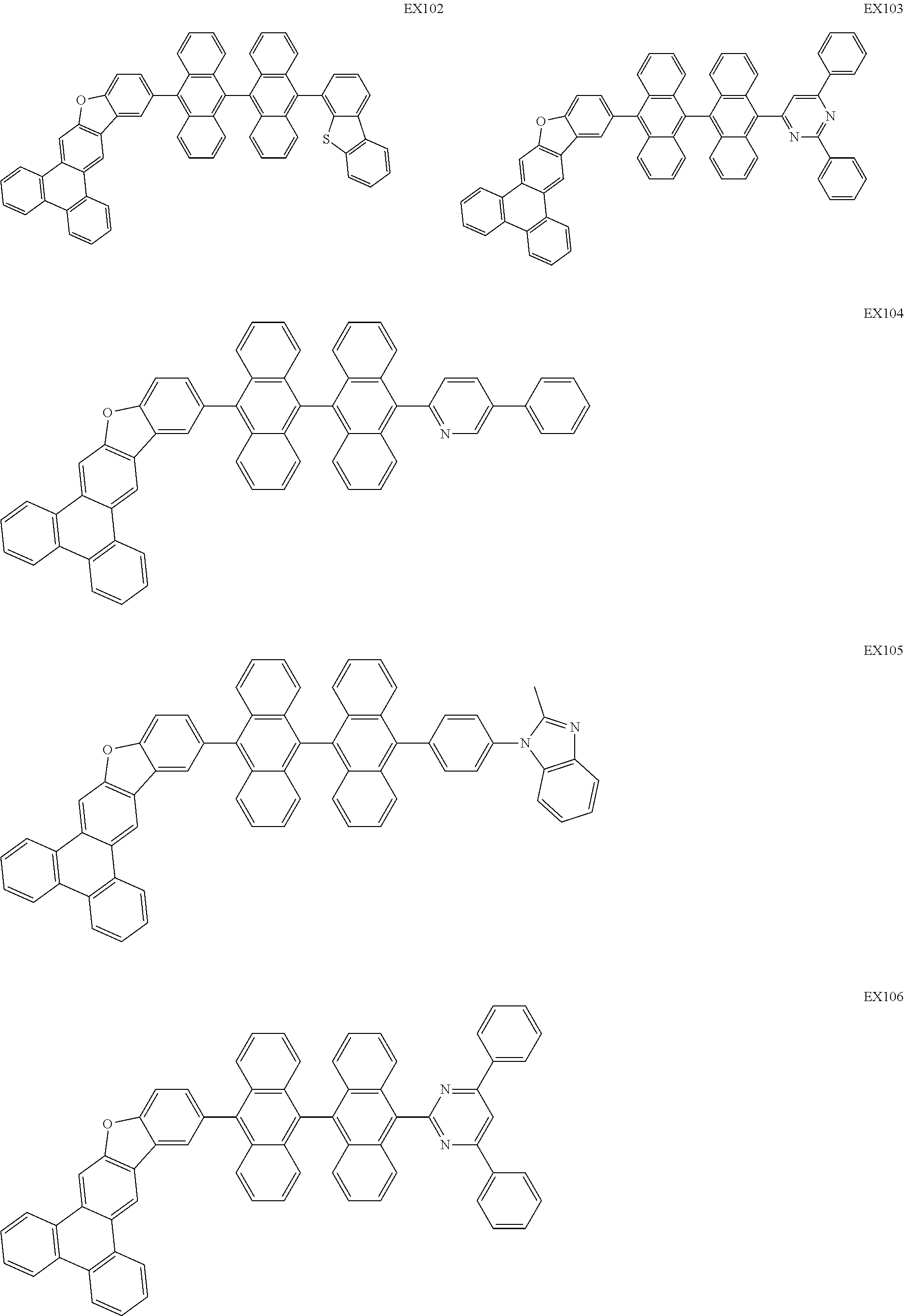

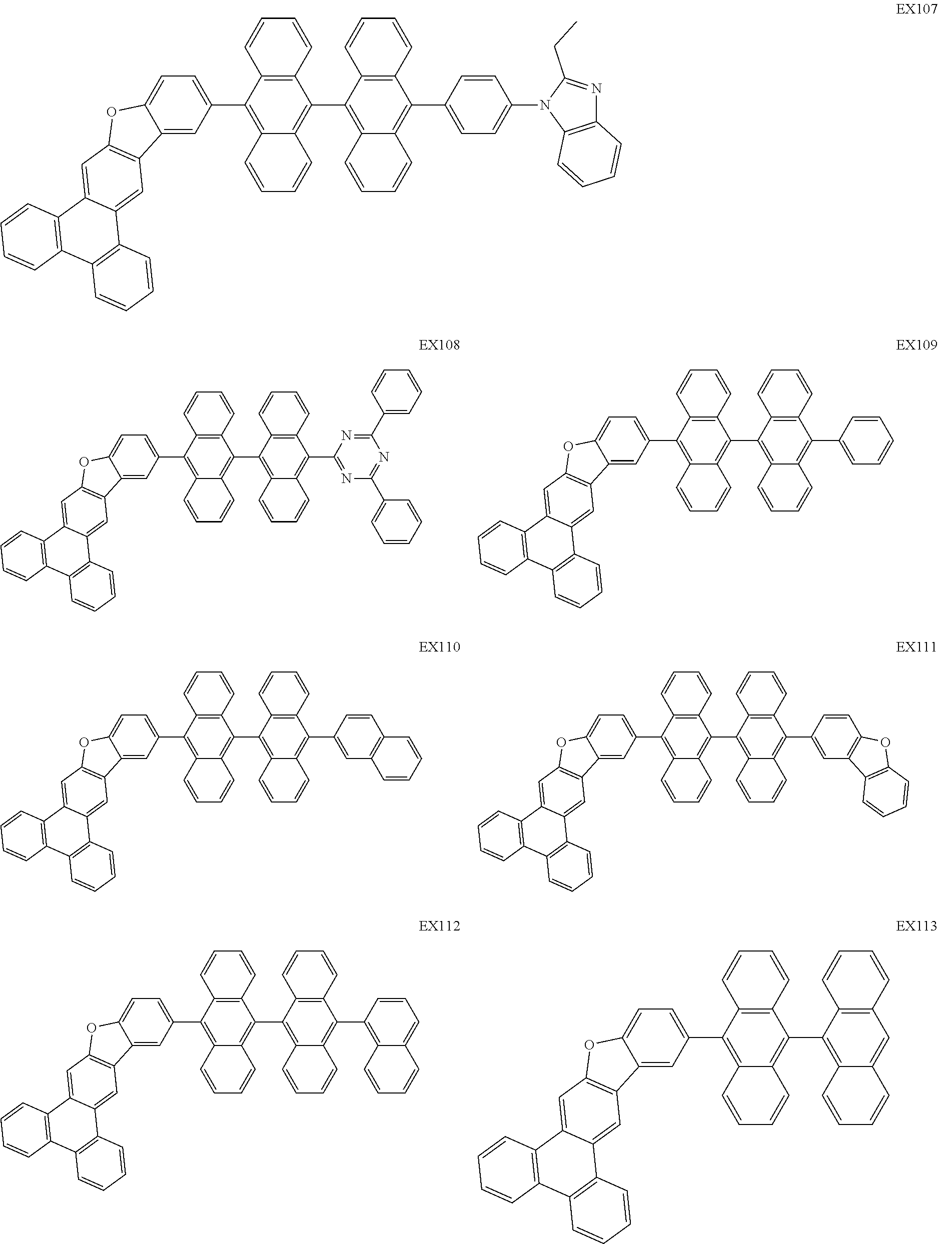

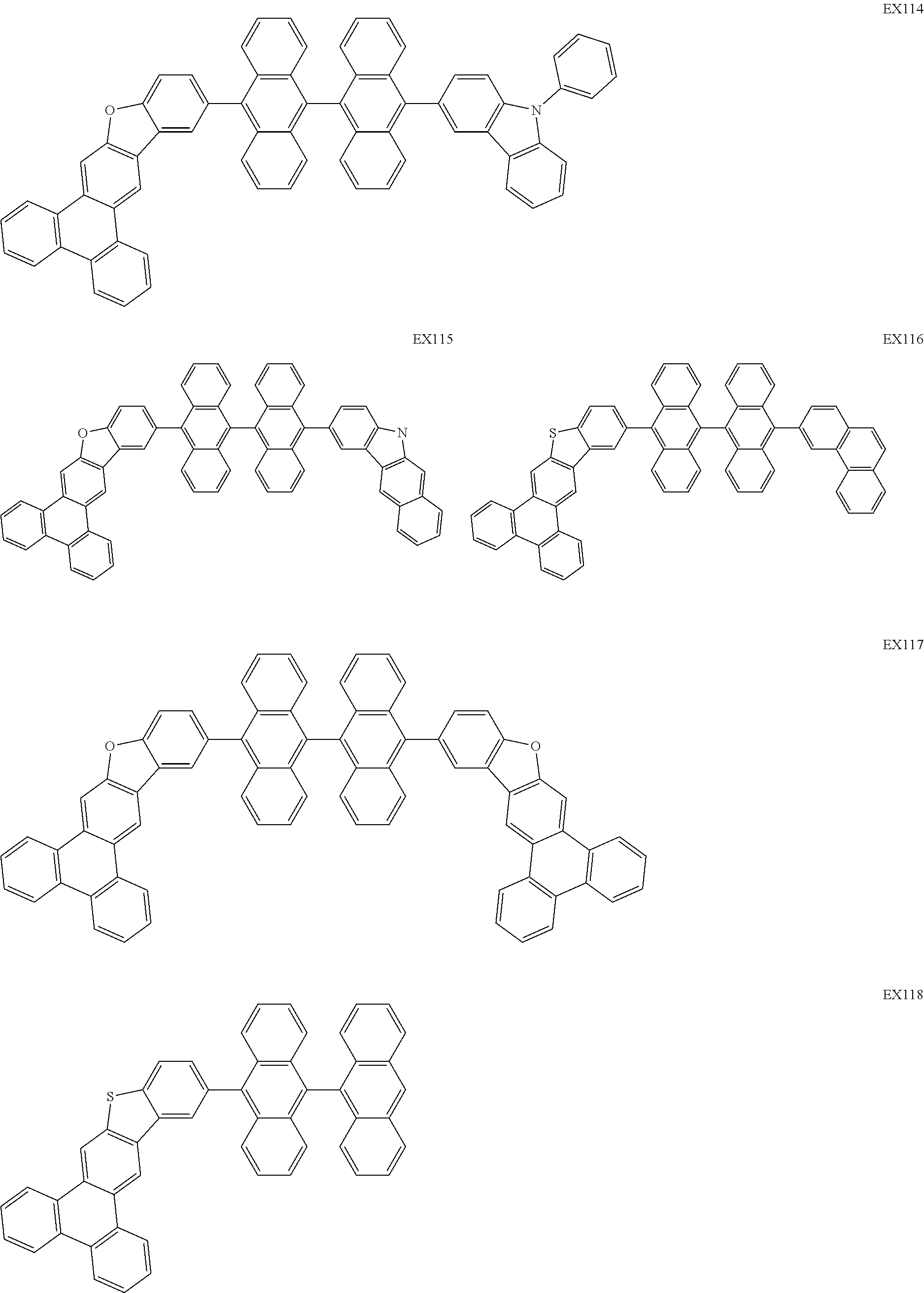

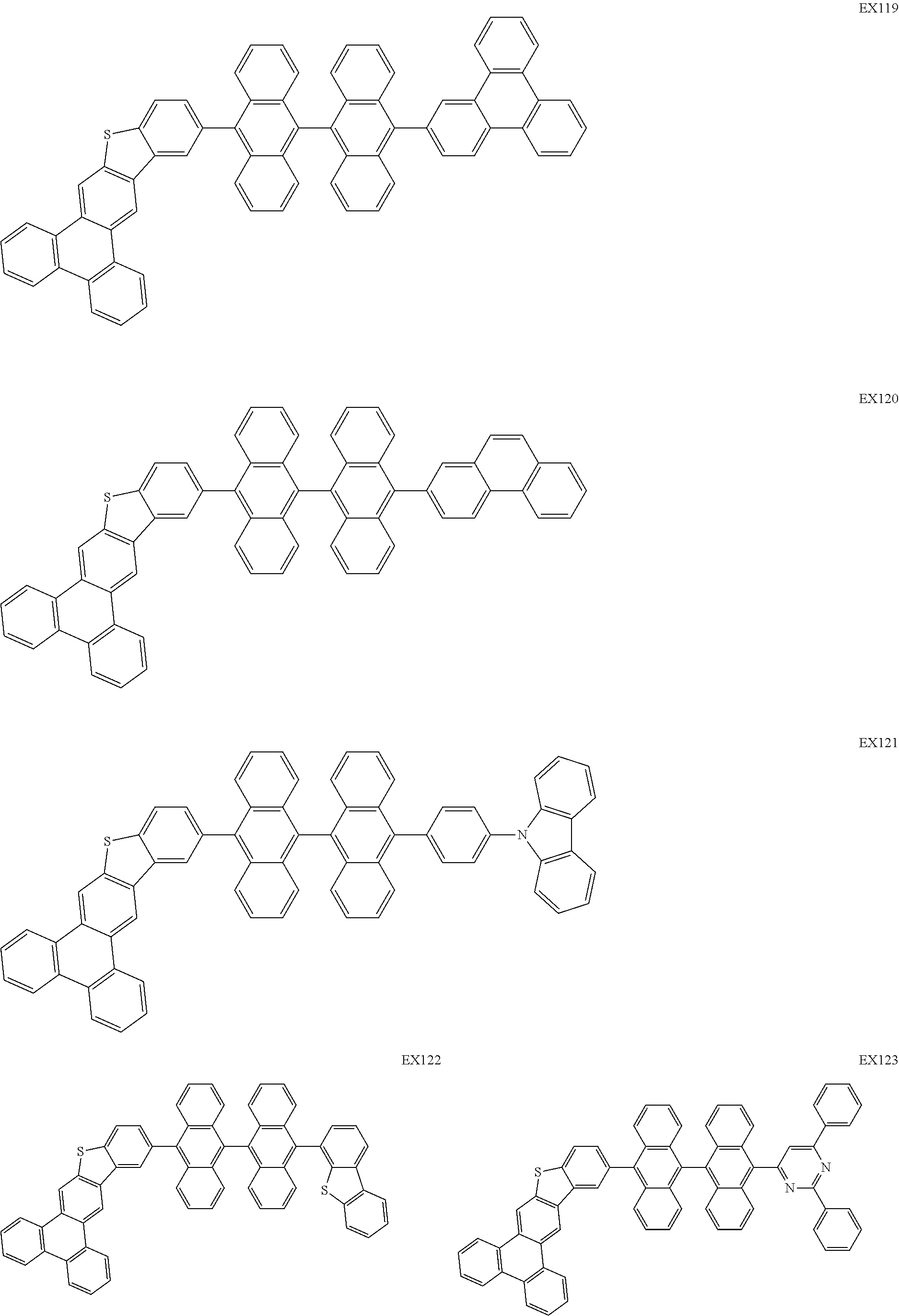

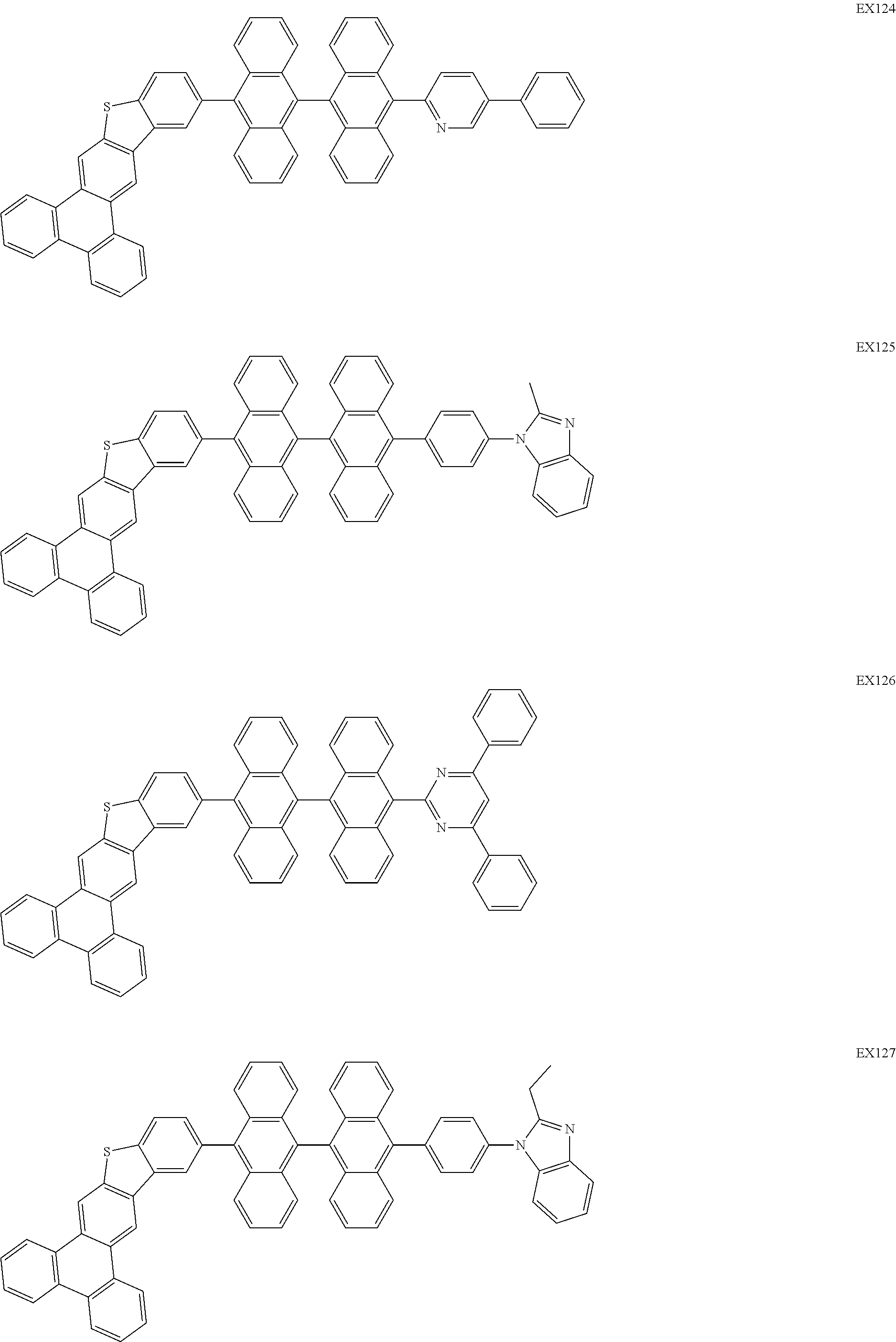 [SMILES: c1ccc(-c2ccc(-c3c4ccccc4c(-c4c5ccccc5c(-c5ccc6sc7cc8c9ccccc9c9ccccc9c8cc7c6c5)c5ccccc45)c4ccccc34)nc2)cc1 ; Cc1nc2ccccc2n1-c1ccc(-c2c3ccccc3c(-c3c4ccccc4c(-c4ccc5sc6cc7c8ccccc8c8ccccc8c7cc6c5c4)c4ccccc34)c3ccccc23)cc1 ; c1ccc(-c2cc(-c3ccccc3)nc(-c3c4ccccc4c(-c4c5ccccc5c(-c5ccc6sc7cc8c9ccccc9c9ccccc9c8cc7c6c5)c5ccccc45)c4ccccc34)n2)cc1 ; CCc1nc2ccccc2n1-c1ccc(-c2c3ccccc3c(-c3c4ccccc4c(-c4ccc5sc6cc7c8ccccc8c8ccccc8c7cc6c5c4)c4ccccc34)c3ccccc23)cc1]

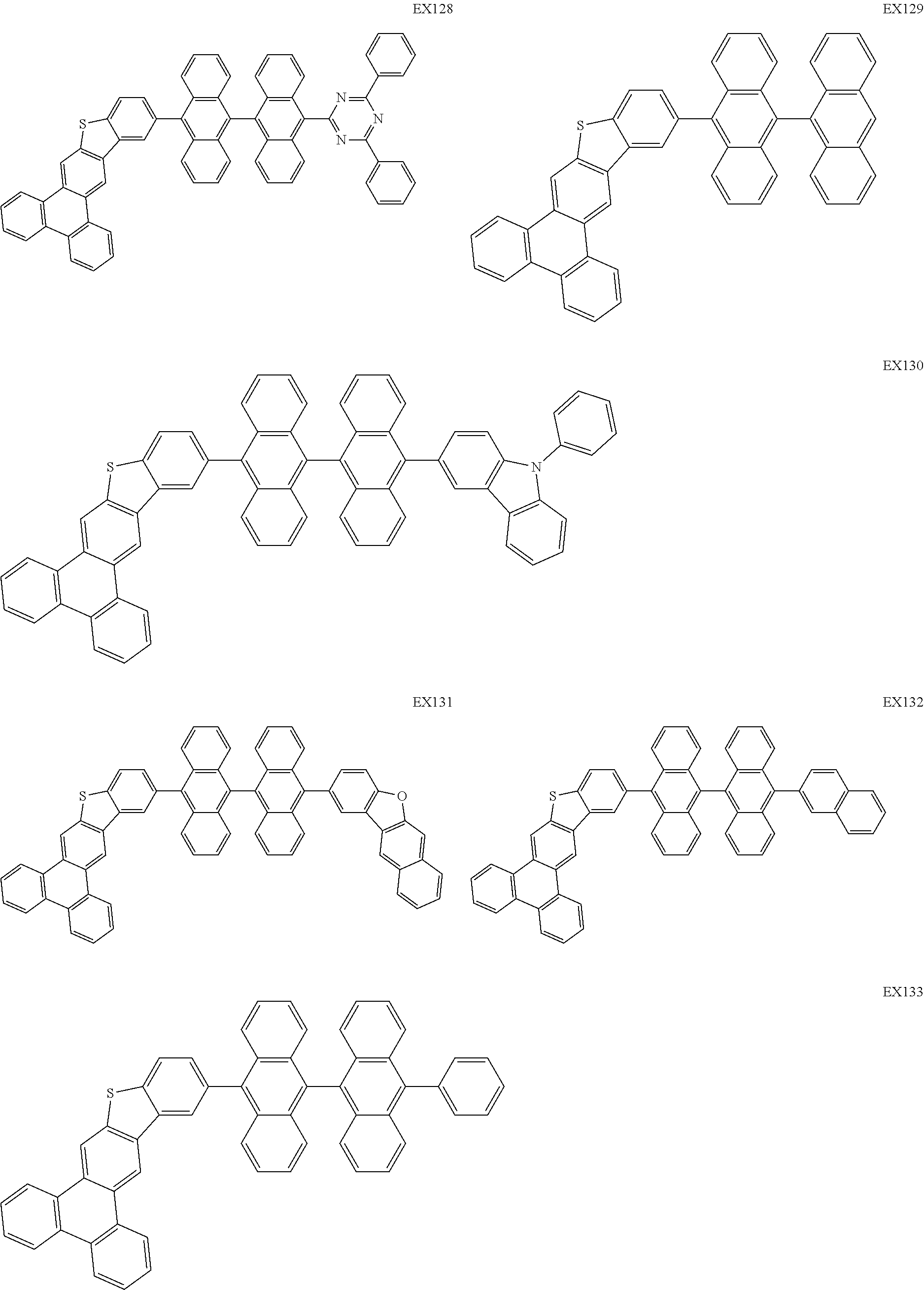

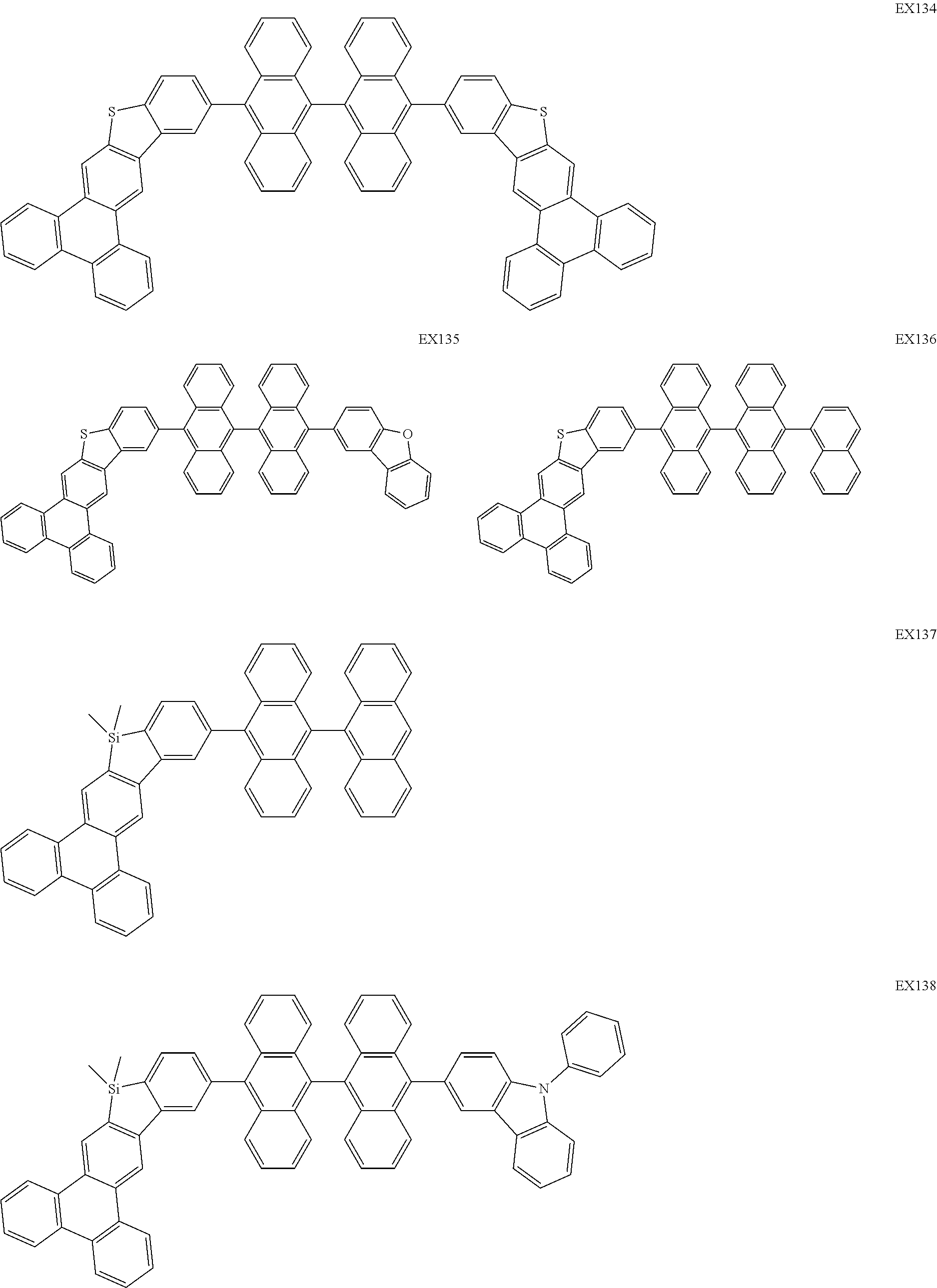

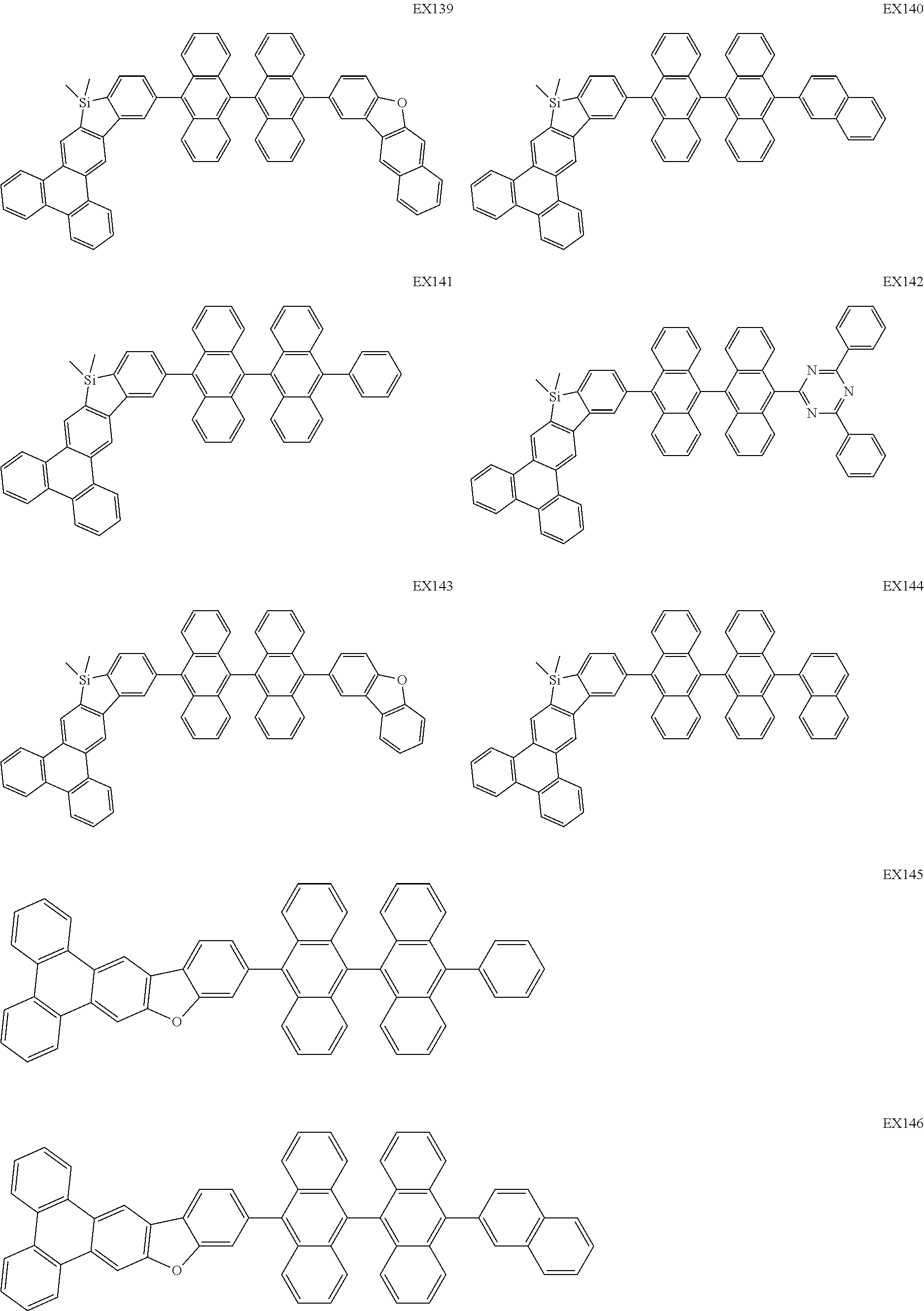

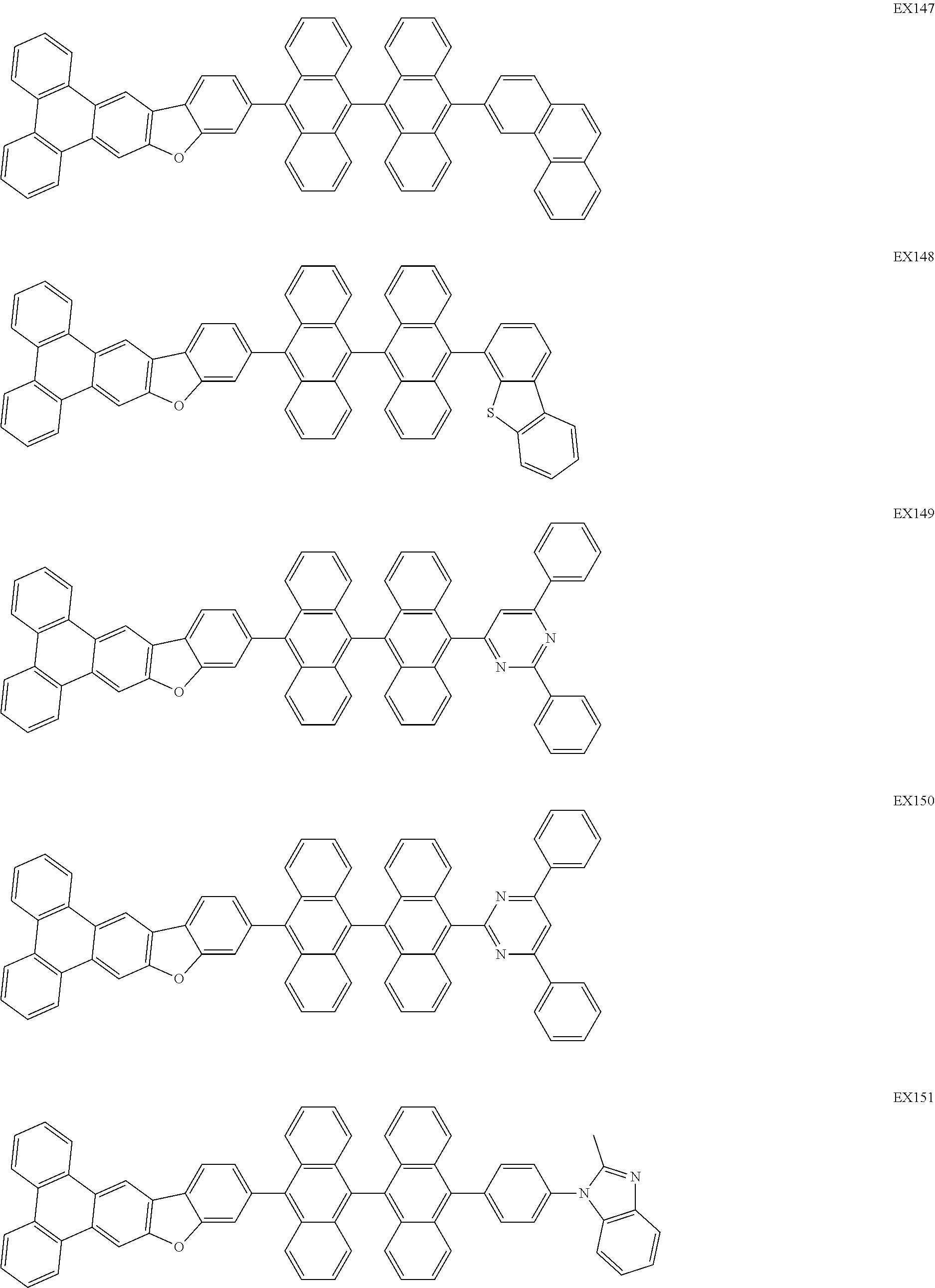

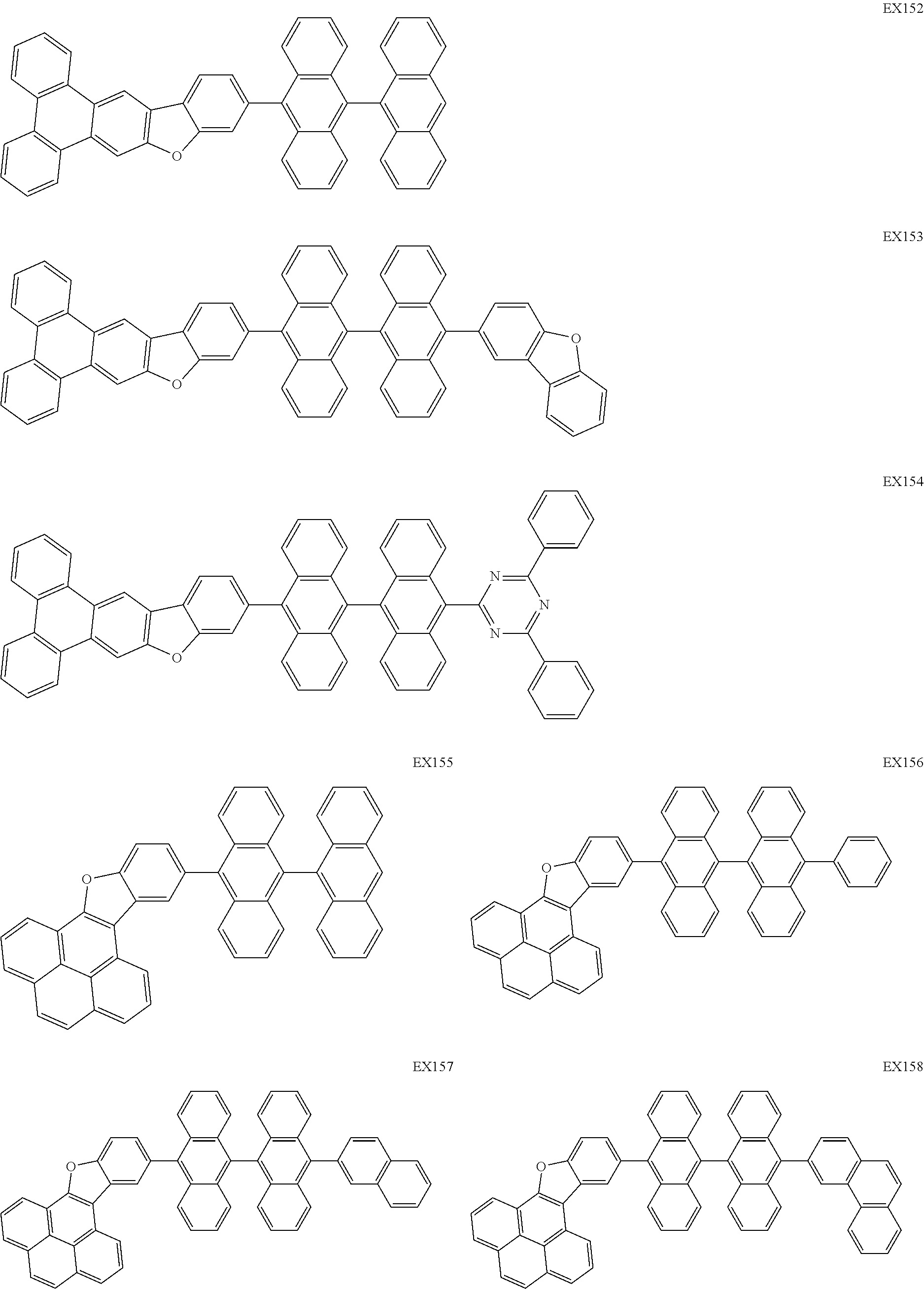

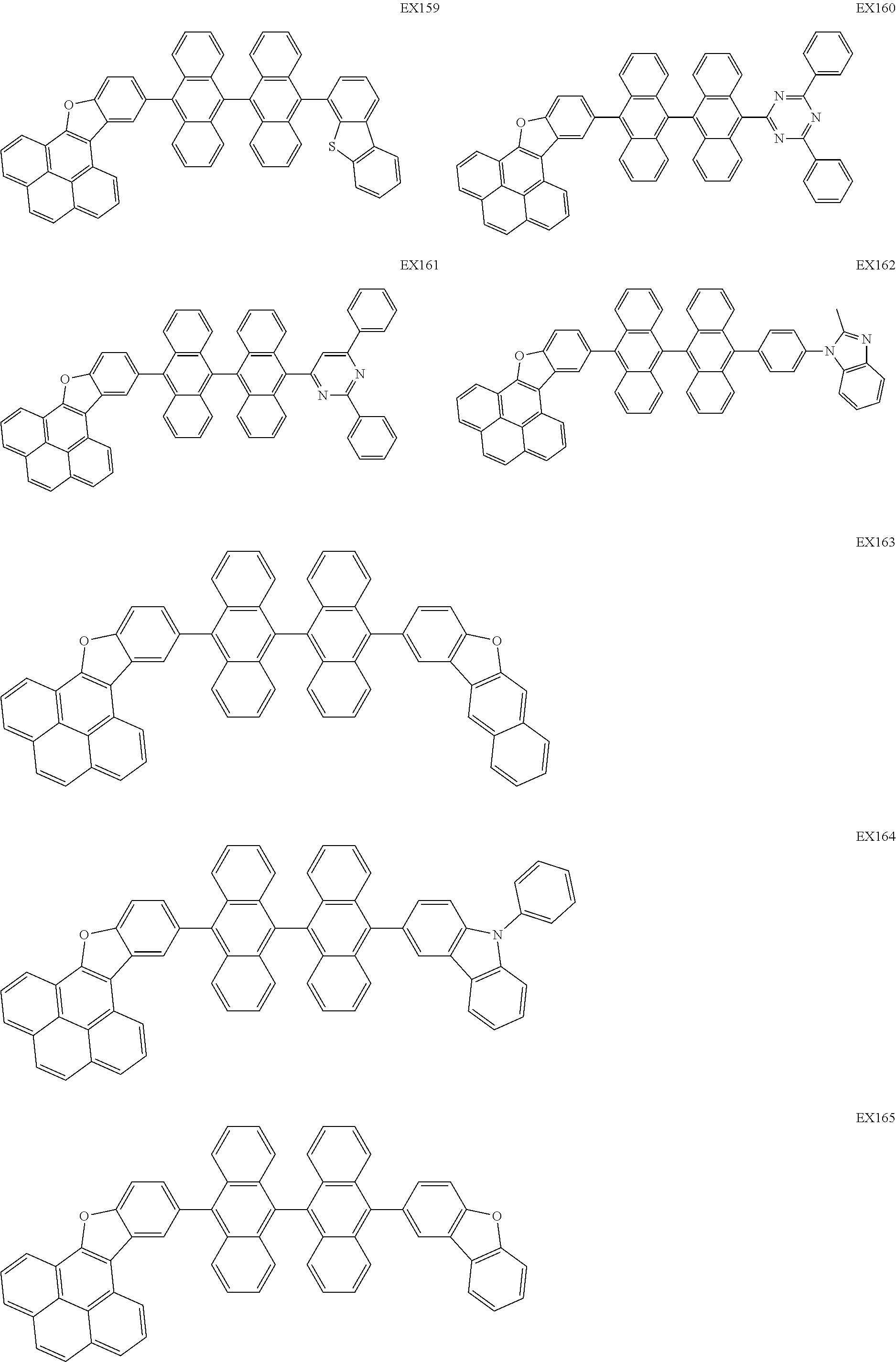

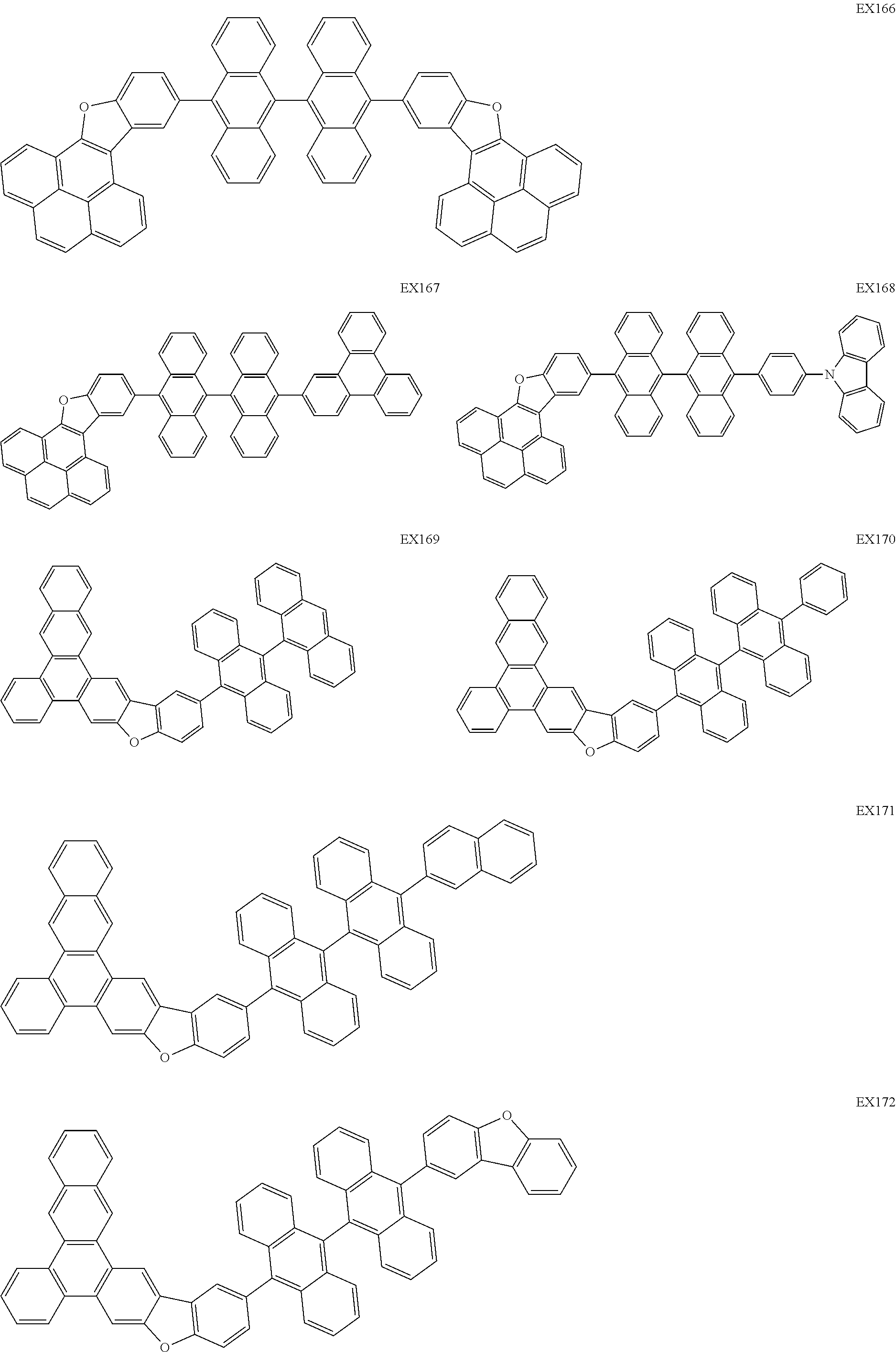

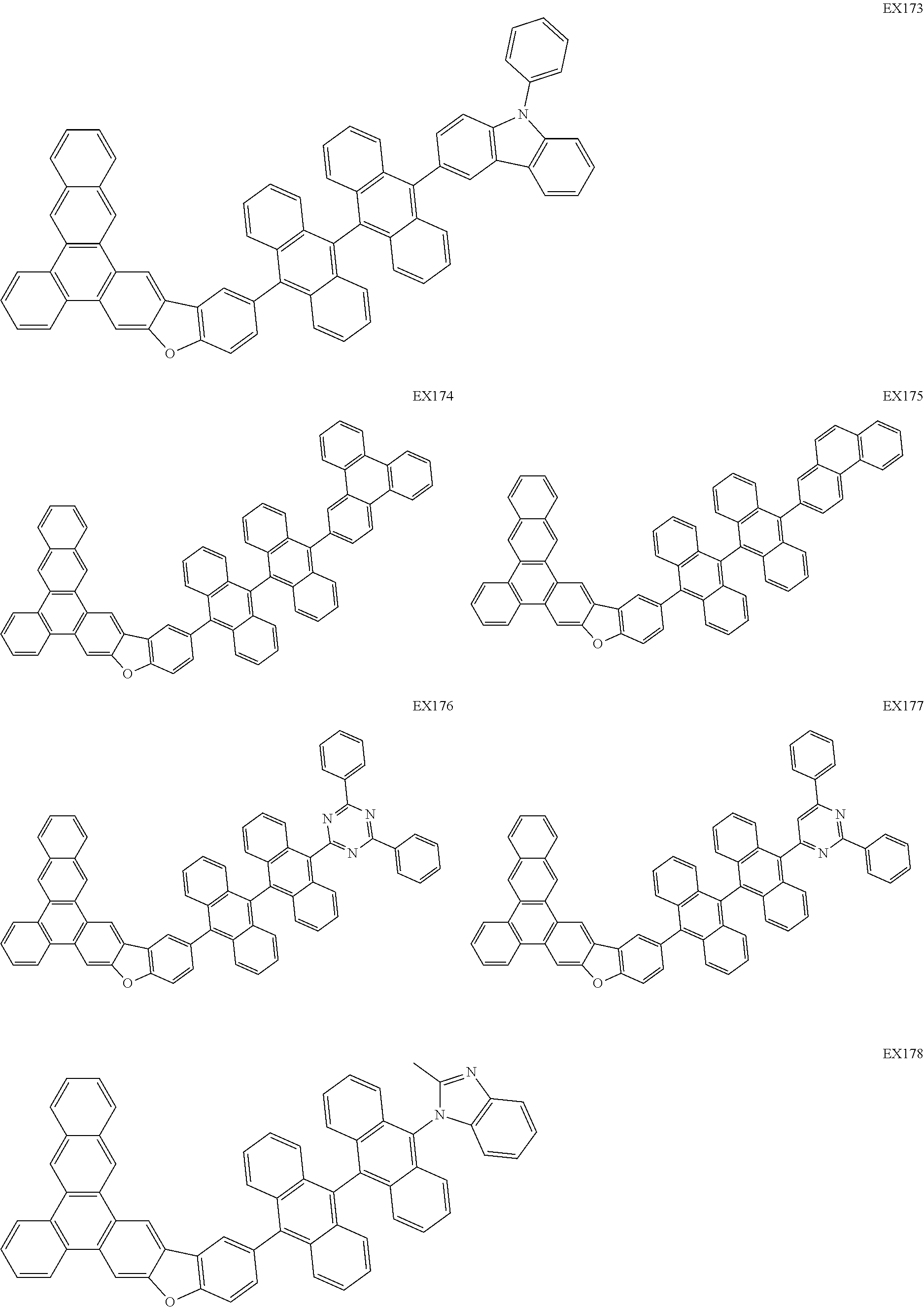

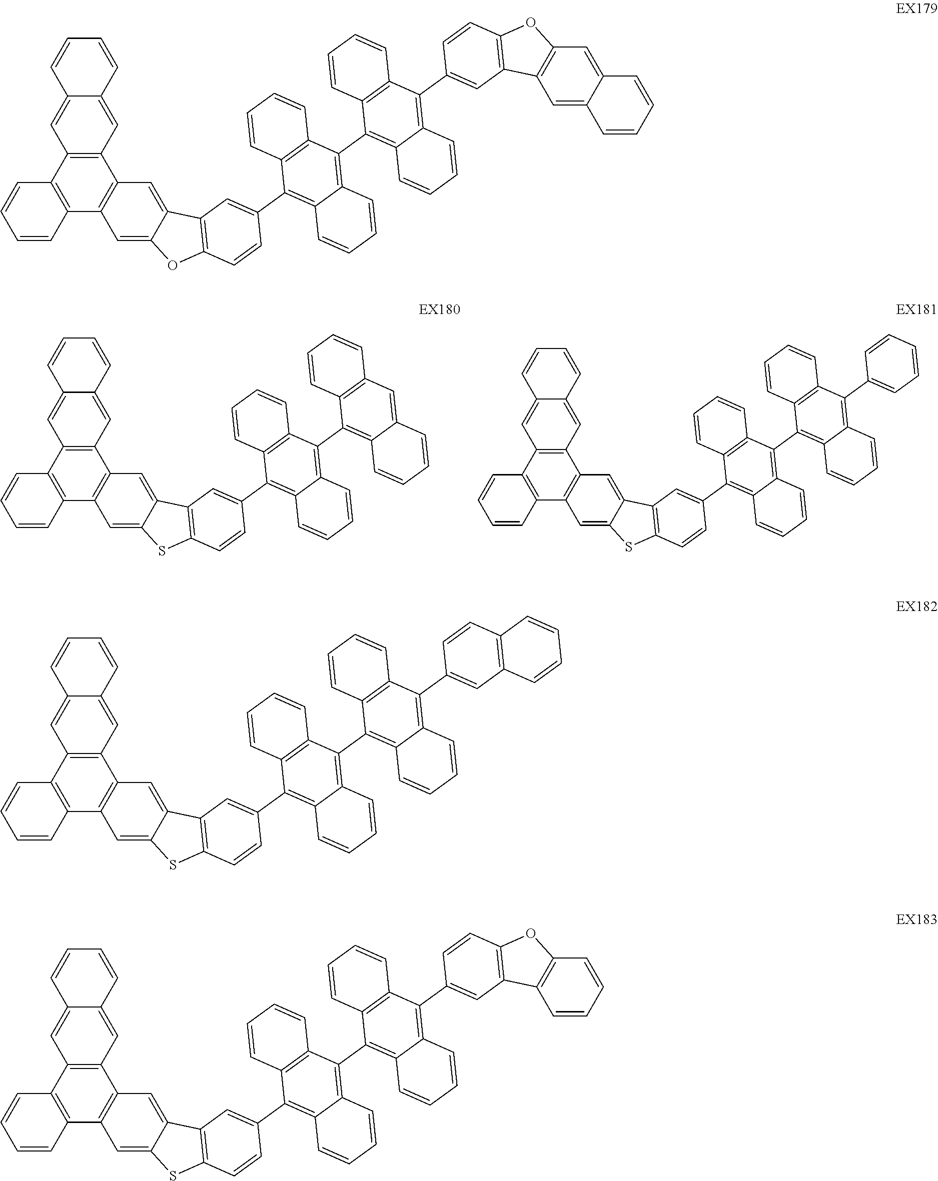

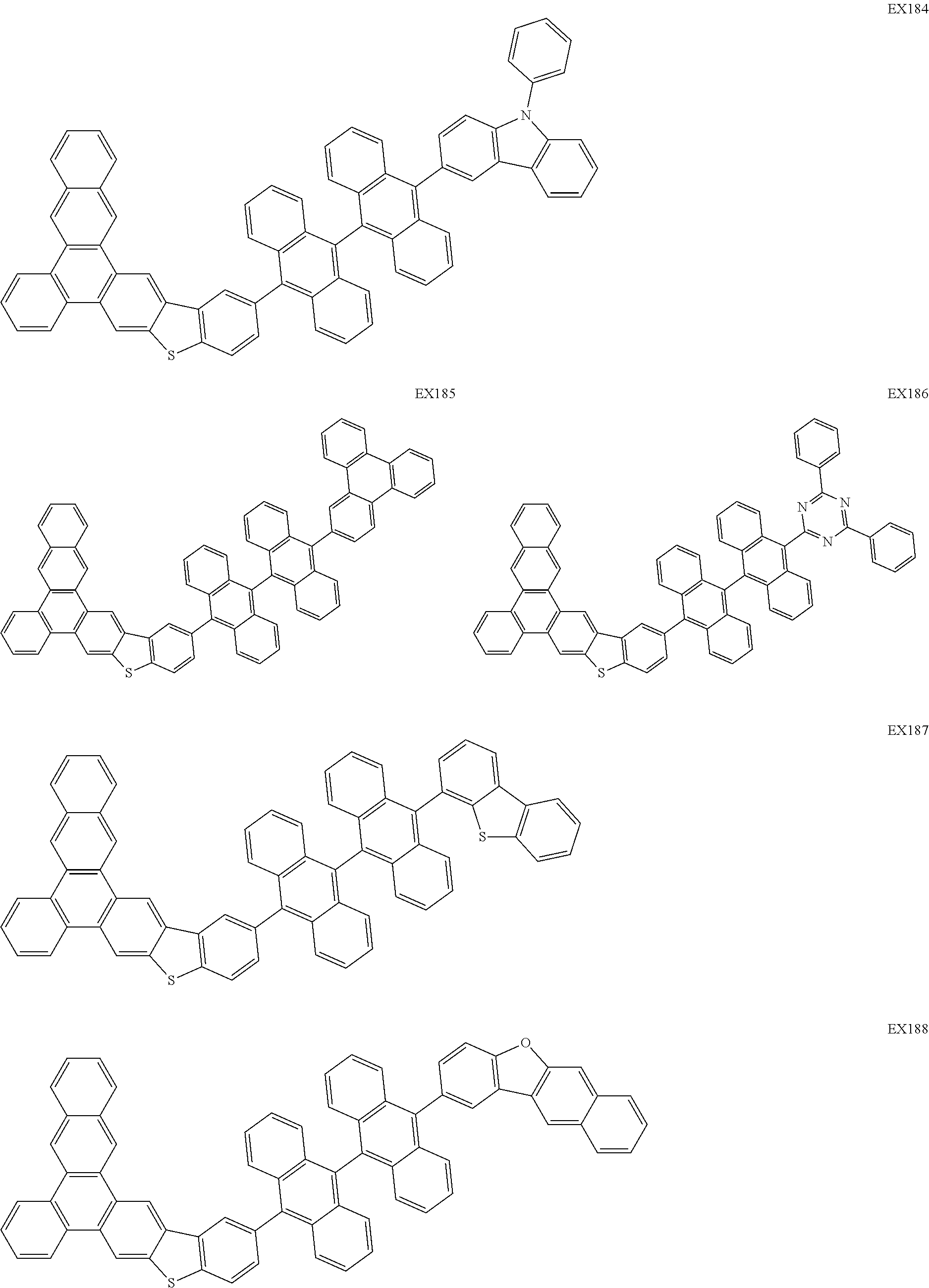

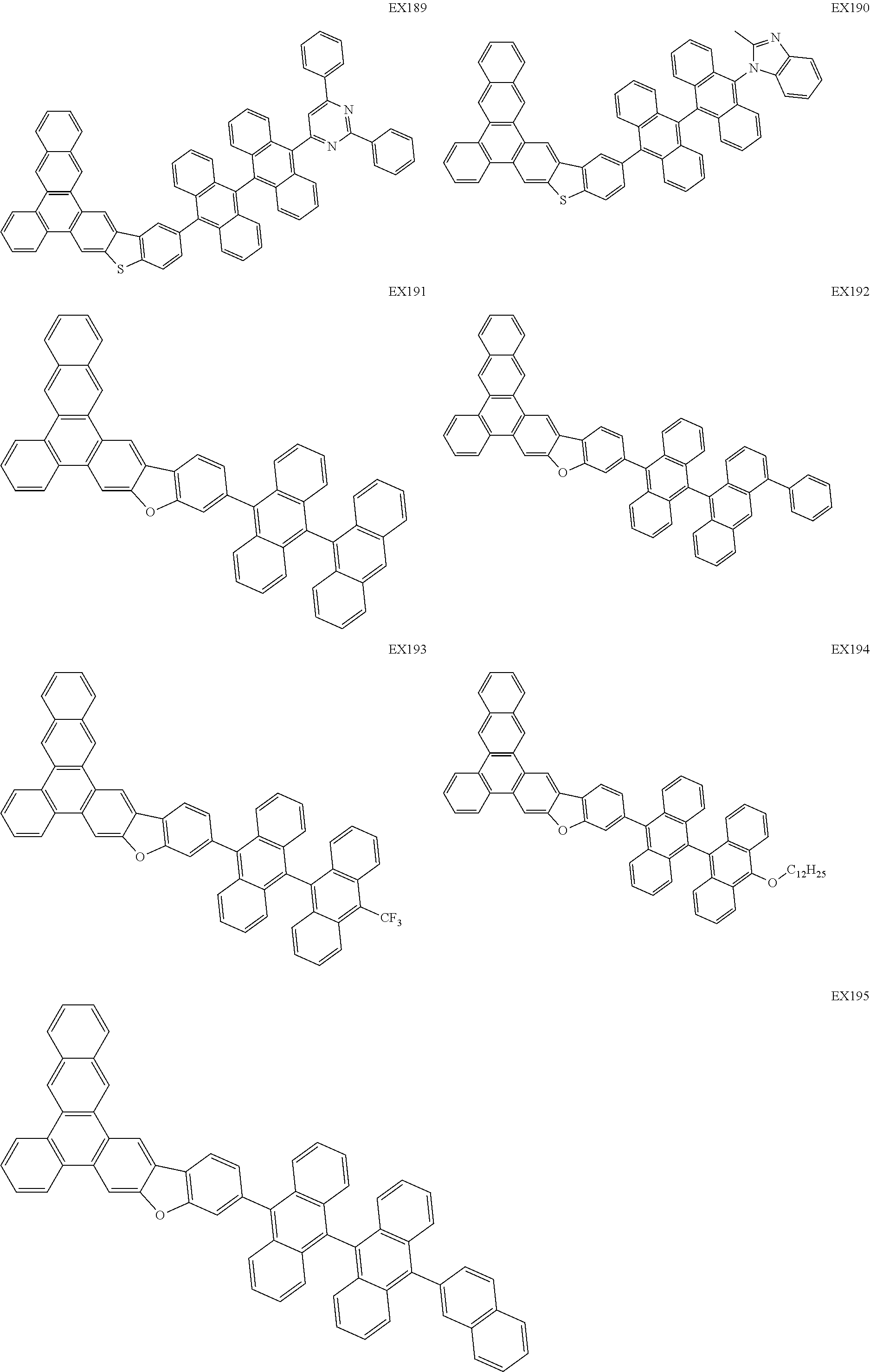

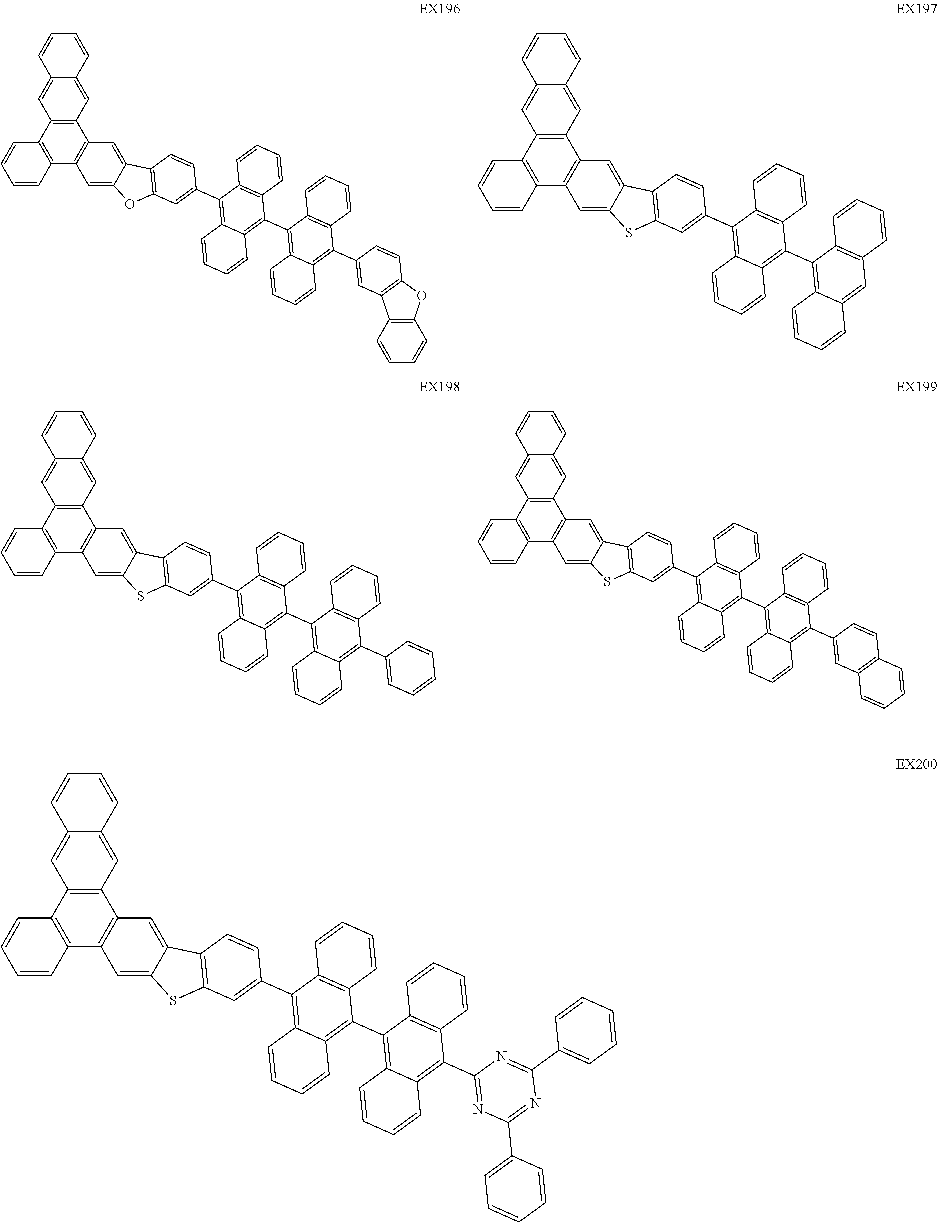

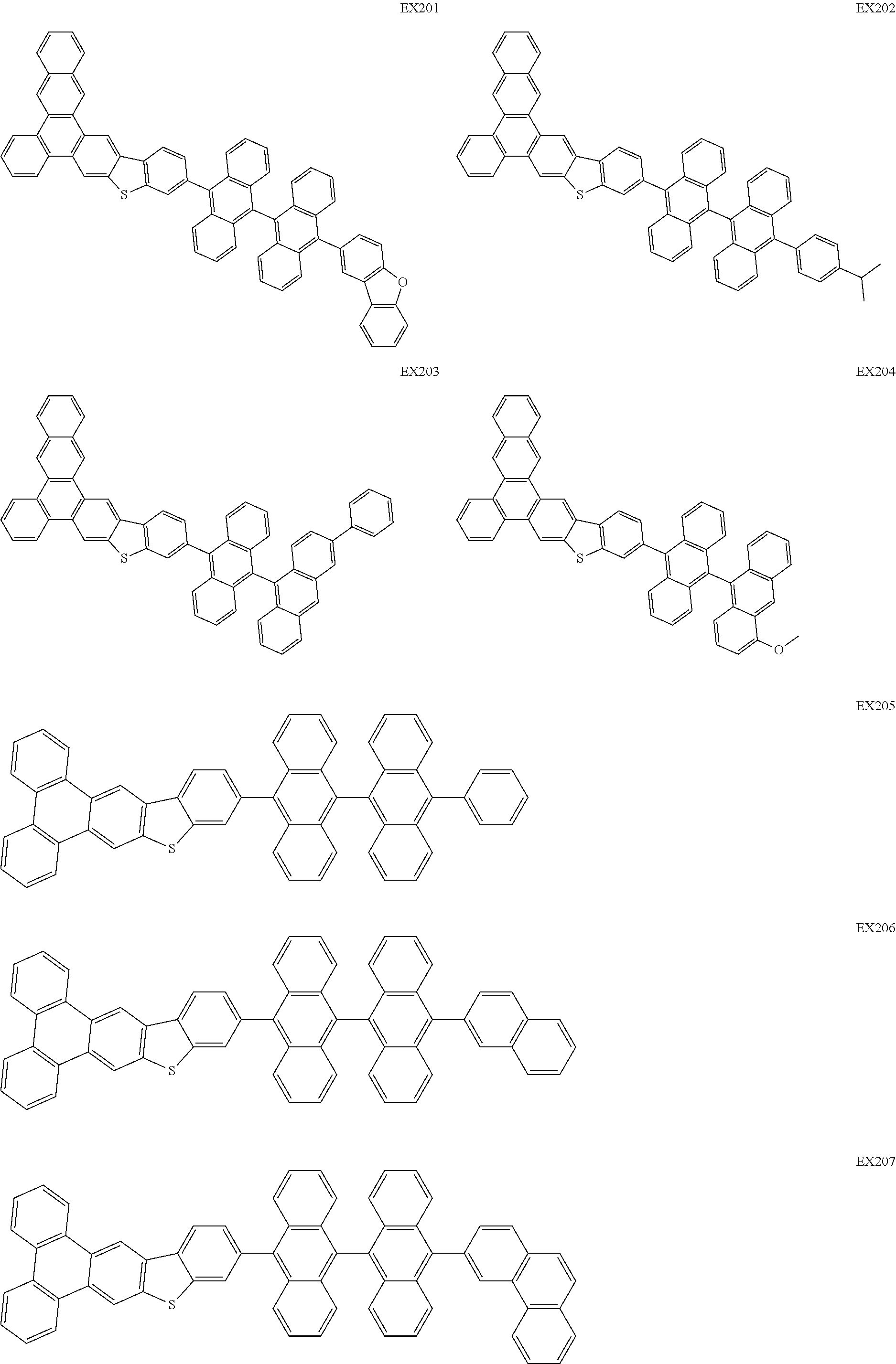

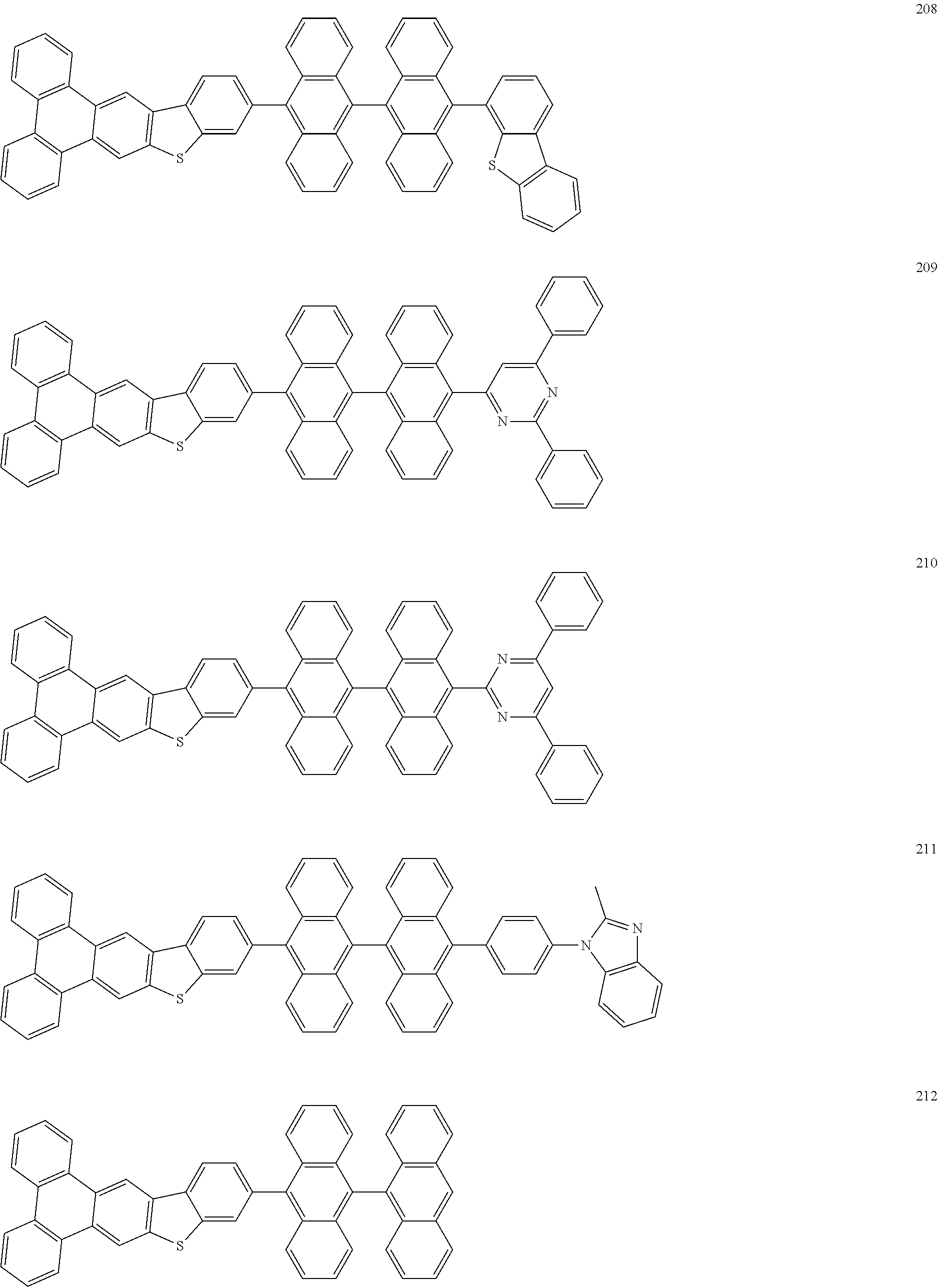

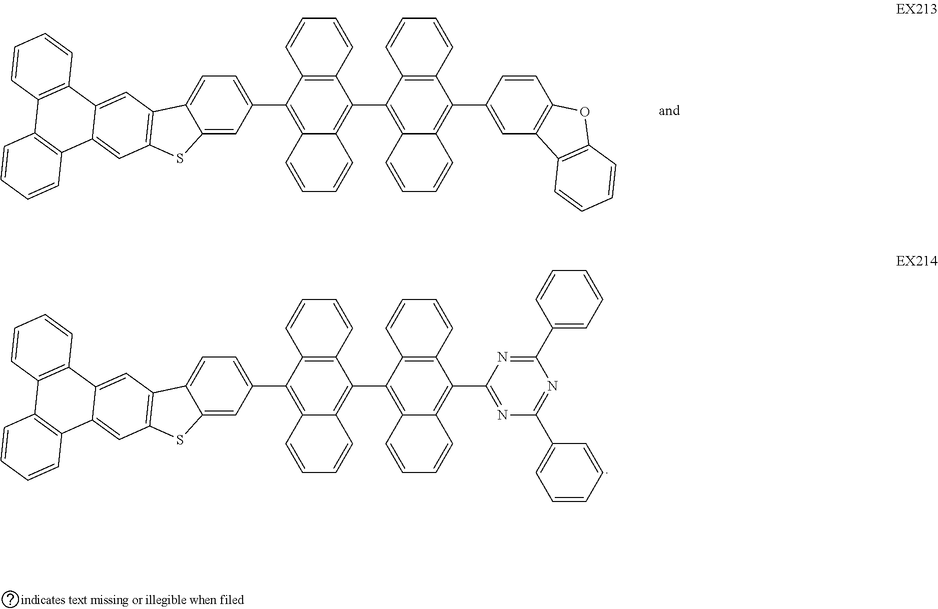 [SMILES: c1ccc2c(c1)oc1ccc(-c3c4ccccc4c(-c4c5ccccc5c(-c5ccc6c(c5)sc5cc7c8ccccc8c8ccccc8c7cc56)c5ccccc45)c4ccccc34)cc12 ; c1ccc(-c2nc(-c3ccccc3)nc(-c3c4ccccc4c(-c4c5ccccc5c(-c5ccc6c(c5)sc5cc7c8ccccc8c8ccccc8c7cc56)c5ccccc45)c4ccccc34)n2)cc1]

In another embodiment of the present invention, an organic electroluminescence device is disclosed. The organic electroluminescence device comprises a pair of electrodes composed of a cathode and an anode, and a light emitting layer and one or more organic thin film layers between the pair of electrodes. At least one of the light emitting layer and the organic thin film layer comprises the organic compound of formula (1).

In some embodiments, the light emitting layer comprising the organic compound of formula (1) is a host material. The host material may be a phosphorescent host material or a fluorescent host material. In certain embodiments, the light emitting layer comprising the organic compound of formula (1) is used as a fluorescent dopant material.

In a further embodiment of the present invention, the organic electroluminescence device is a lighting panel. In other embodiment of the present invention, the organic electroluminescence device is a backlight panel.

Detailed preparation of the organic compounds of the present invention will be clarified by exemplary embodiments below, but the present invention is not limited thereto. EXAMPLES 1 to 16 show the preparation of the organic compounds of the present invention, and EXAMPLES 17 show the fabrication and test reports of the organic EL devices.

### EXAMPLE 1

### Synthesis of EX1

### Synthesis of Intermediate A

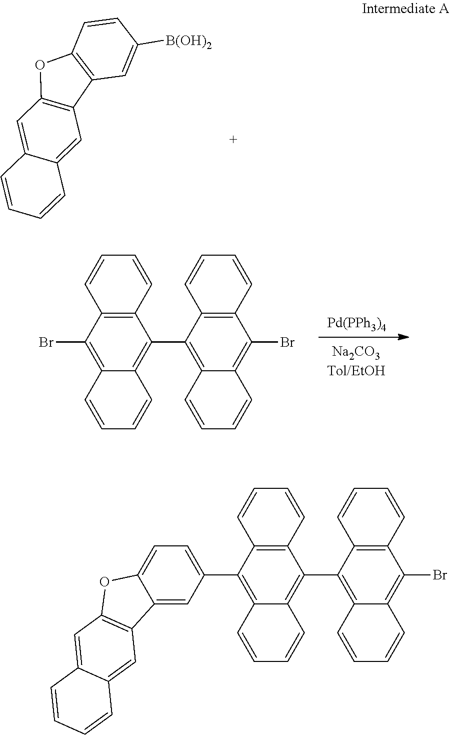 [SMILES: OB(O)c1ccc2oc3cc4ccccc4cc3c2c1 ; Brc1c2ccccc2c(-c2c3ccccc3c(Br)c3ccccc23)c2ccccc12 ; Brc1c2ccccc2c(-c2c3ccccc3c(-c3ccc4oc5cc6ccccc6cc5c4c3)c3ccccc23)c2ccccc12 ; "Pd(PPh3)4"]

A mixture of 8.2 g (16.0 mmole) of 10,10′-Dibromo-9,9′-bianthracene, 5.0 g (19.1 mmole) of Naphtho[2,3-b]benzofuran-2-ylboronic acid, 0.4 g (0.3 mmole) of Pd(PPh₃)₄, 0.2 g (0.6 mmole) of 2-Dicyclophosphine-2′,6′-dimethoxy-biphenyl, 2.5 g (24.0 mmole) of Na₂CO₃, 120 ml of Toluene and 40 ml of Ethanol, and 12 ml of H₂O was placed under nitrogen, and then heated at 80° C. while stirring for 16 h. After the reaction finished, the mixture was allowed to cool to room temperature. The solution was extracted with 100 ml of ethyl acetate (3 times) and then 300 ml of water. The organic layer was dried with anhydrous magnesium sulfate, and then the solvent was evaporated under reduced pressure. The residue was purified by column chromatography on silica to give product (7.8 g, 75%) as a white solid.

### Synthesis of EX1

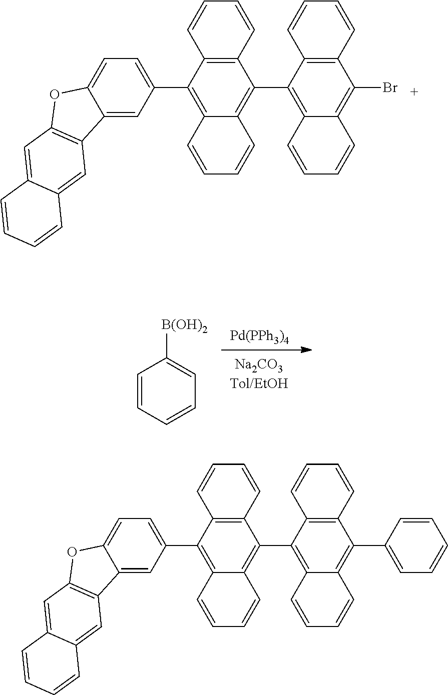 [SMILES: Brc1c2ccccc2c(-c2c3ccccc3c(-c3ccc4oc5cc6ccccc6cc5c4c3)c3ccccc23)c2ccccc12 ; OB(O)c1ccccc1 ; c1ccc(-c2c3ccccc3c(-c3c4ccccc4c(-c4ccc5oc6cc7ccccc7cc6c5c4)c4ccccc34)c3ccccc23)cc1 ; "Pd(PPh3)4"]

A mixture of 7.8 g (12.0 mmole) of Intermediate A, 1.75 g (14.4 mmole) of Phenylboronic acid, 0.27 g (0.24 mmole) of Pd(PPh₃)₄, 0.17 g (0.48 mmole) of 2-Dicyclophosphine-2′,6′-dimethoxybiphenyl, 1.27 g (18.0 mmole) of Na₂CO₃, 120 ml of Toluene and 40 ml of Ethanol, and 9 ml of H₂O was placed under nitrogen, and then heated at 80° C. while stirring for 16 h. After the reaction finished, the mixture was allowed to cool to room temperature. Then 700 ml of MeOH was added while stirring and the precipitated product was filtered off with suction to give (3.3 g, 63%) of off-white product, which was recrystallized from EtOH. MS(m/z, EI⁺):647.7

### EXAMPLE 2

### Synthesis of EX2

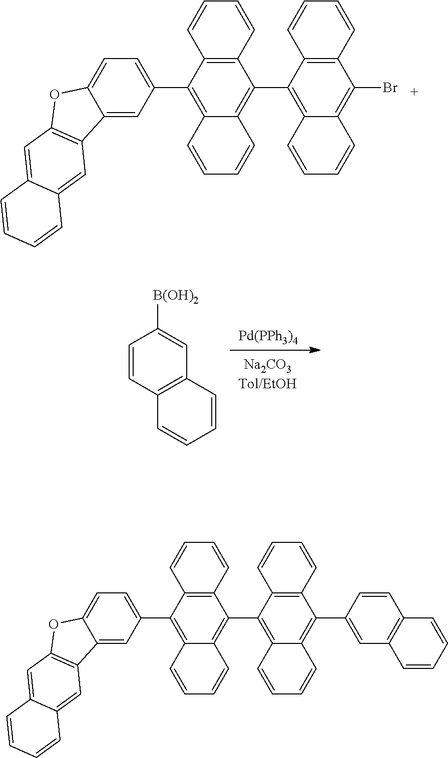 [SMILES: Brc1c2ccccc2c(-c2c3ccccc3c(-c3ccc4oc5cc6ccccc6cc5c4c3)c3ccccc23)c2ccccc12 ; OB(O)c1ccc2ccccc2c1 ; c1ccc2cc(-c3c4ccccc4c(-c4c5ccccc5c(-c5ccc6oc7cc8ccccc8cc7c6c5)c5ccccc45)c4ccccc34)ccc2c1 ; "Pd(PPh3)4"]

A mixture of 9.0 g (13.8 mmole) of Intermediate A, 2.8 g (16.6 mmole) of 2-Naphthylboronic acid, 0.32 g (0.28 mmole) of Pd(PPh₃)₄, 0.20 g (0.55 mmole) of 2-Dicyclophosphine-2′,6′-dimethoxybiphenyl, 2.19 g (20.7 mmole) of Na₂CO₃, 135 ml of Toluene and 45 ml of Ethanol, and 11 ml of H₂O was placed under nitrogen, and then heated at 80° C. while stirring for 16 h. After the reaction finished, the mixture was allowed to cool to room temperature. Then 700 ml of MeOH was added while stirring and the precipitated product was filtered off with suction to give (6.3g, 66%) of off-white product, which was recrystallized from EtOH. MS(m/z, EI⁺):697.8

### EXAMPLE 3

### Synthesis of EX5

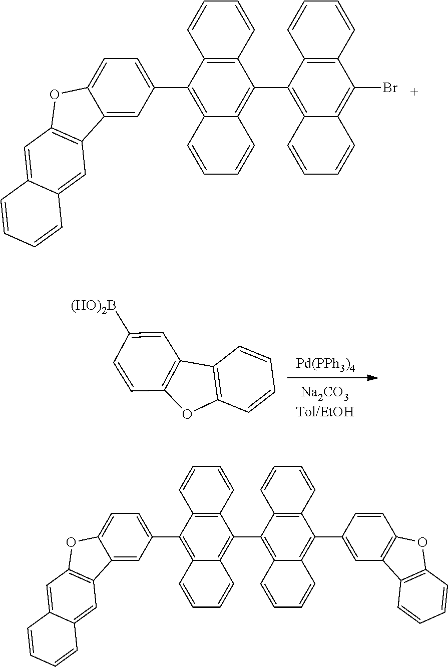 [SMILES: Brc1c2ccccc2c(-c2c3ccccc3c(-c3ccc4oc5cc6ccccc6cc5c4c3)c3ccccc23)c2ccccc12 ; OB(O)c1ccc2oc3ccccc3c2c1 ; c1ccc2cc3c(cc2c1)oc1ccc(-c2c4ccccc4c(-c4c5ccccc5c(-c5ccc6oc7ccccc7c6c5)c5ccccc45)c4ccccc24)cc13 ; "Pd(PPh3)4"]

A mixture of 9.0 g (13.8 mmole) of Intermediate A, 3.5 g (16.6 mmole) of Dibenzo[b,d]furan-2-ylboronic acid, 0.32 g (0.28 mmole) of Pd(PPh₃)₄, 0.20 g (0.55 mmole) of 2-Dicyclophosphine-2′,6′-dimethoxybiphenyl, 2.19 g (20.7 mmole) of Na₂CO₃, 135 ml of Toluene and 45 ml of Ethanol, and 11 ml of H₂O was placed under nitrogen, and then heated at 80° C. while stirring for 16 h. After the reaction finished, the mixture was allowed to cool to room temperature. Then 700 ml of MeOH was added while stirring and the precipitated product was filtered off with suction to give (6.5 g, 64%) of off-white product, which was recrystallized from EtOH. MS(m/z, EI⁺):737.8

### EXAMPLE 4

### Synthesis of EX7

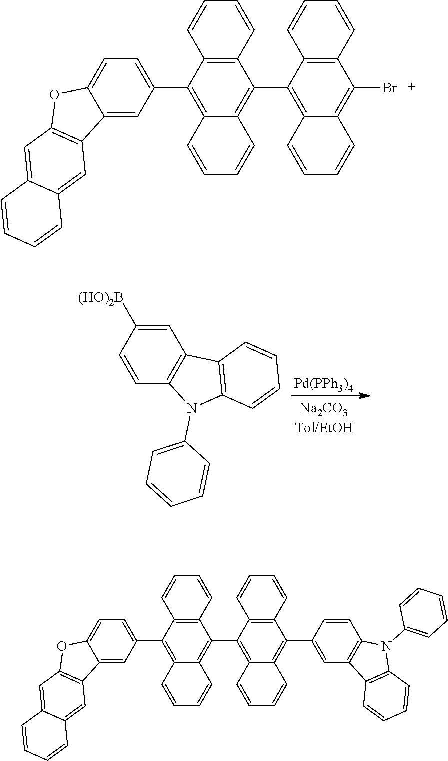 [SMILES: Brc1c2ccccc2c(-c2c3ccccc3c(-c3ccc4oc5cc6ccccc6cc5c4c3)c3ccccc23)c2ccccc12 ; OB(O)c1ccc2c(c1)c1ccccc1n2-c1ccccc1 ; c1ccc(-n2c3ccccc3c3cc(-c4c5ccccc5c(-c5c6ccccc6c(-c6ccc7oc8cc9ccccc9cc8c7c6)c6ccccc56)c5ccccc45)ccc32)cc1 ; "Pd(PPh3)4"]

A mixture of 9.0 g (13.8 mmole) of Intermediate A, 4.7 g (16.6 mmole) of 9-Phenyl-9H-carbazol-3-ylboronic acid, 0.32 g (0.28 mmole) of Pd(PPh₃)₄, 0.20 g (0.55 mmole) of 2-Dicyclophosphine-2′,6′-dimethoxybiphenyl, 2.19 g (20.7 mmole) of Na₂CO₃, 135 ml of Toluene and 45 ml of Ethanol, and 11 ml of H₂O was placed under nitrogen, and then heated at 80° C. while stirring for 16 h. After the reaction finished, the mixture was allowed to cool to room temperature. Then 700 ml of MeOH was added while stirring and the precipitated product was filtered off with suction to give (8.0 g, 71%) of off-white product, which was recrystallized from EtOH. MS(m/z, EI⁺):812.9

### EXAMPLE 5

### Synthesis of EX16

### Synthesis of Intermediate B

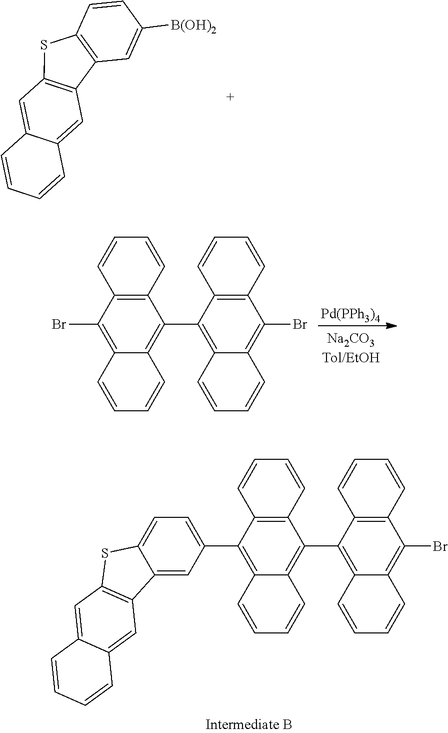 [SMILES: OB(O)c1ccc2sc3cc4ccccc4cc3c2c1 ; Brc1c2ccccc2c(-c2c3ccccc3c(Br)c3ccccc23)c2ccccc12 ; Brc1c2ccccc2c(-c2c3ccccc3c(-c3ccc4sc5cc6ccccc6cc5c4c3)c3ccccc23)c2ccccc12 ; "Pd(PPh3)4"]

A mixture of 10.8 g (21.0 mmole) of 10,10′-Dibromo-9,9′-bianthracene, 7.0 g (25.2 mmole) of Benzo[b]naphtho[2,3-d]thiophen-2-ylboronic acid, 0.5 g (0.42 mmole) of Pd(PPh₃)₄, 0.3 g (0.84 mmole) of 2-Dicyclophosphine-2′,6′-dimethoxy-biphenyl, 3.3 g (31.5 mmole) of Na₂CO₃, 160 ml of Toluene and 55 ml of Ethanol, and 16 ml of H₂O was placed under nitrogen, and then heated at 80° C. while stirring for 16 h. After the reaction finished, the mixture was allowed to cool to room temperature. The solution was extracted with 100 ml of ethyl acetate (3 times) and then 300 ml of water. The organic layer was dried with anhydrous magnesium sulfate, and then the solvent was evaporated under reduced pressure. The residue was purified by column chromatography on silica to give product (10.2 g, 73%) as a white solid.

### Synthesis of EX16

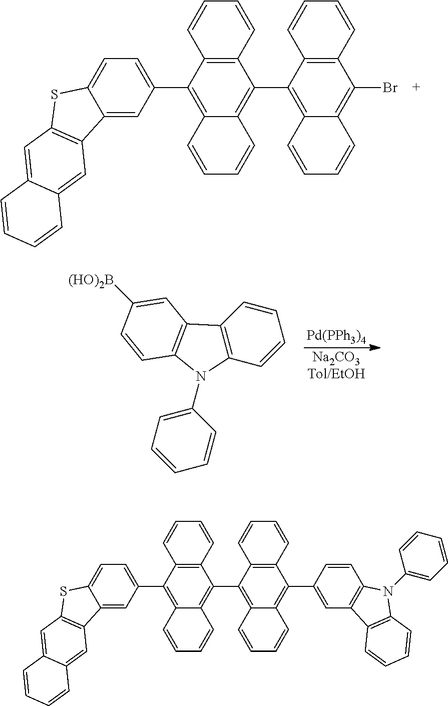 [SMILES: Brc1c2ccccc2c(-c2c3ccccc3c(-c3ccc4sc5cc6ccccc6cc5c4c3)c3ccccc23)c2ccccc12 ; OB(O)c1ccc2c(c1)c1ccccc1n2-c1ccccc1 ; "Na2CO3Tol/EtOH" ; c1ccc(-n2c3ccccc3c3cc(-c4c5ccccc5c(-c5c6ccccc6c(-c6ccc7sc8cc9ccccc9cc8c7c6)c6ccccc56)c5ccccc45)ccc32)cc1 ; "Pd(PPh3)4"]

A mixture of 10.2 g (15.3 mmole) of Intermediate B, 5.3 g (18.4 mmole) of 9-Phenyl-9H-carbazol-3-ylboronic acid, 0.36 g (0.31 mmole) of Pd(PPh₃)₄, 0.21 g (0.61 mmole) of 2-Dicyclophosphine-2′,6′-dimethoxybiphenyl, 2.43 g (23.0 mmole) of Na₂CO₃, 135 ml of Toluene and 45 ml of Ethanol, and 12 ml of H₂O was placed under nitrogen, and then heated at 80° C. while stirring for 16 h. After the reaction finished, the mixture was allowed to cool to room temperature. Then 700 ml of MeOH was added while stirring and the precipitated product was filtered off with suction to give (8.5 g, 67%) of off-white product, which was recrystallized from EtOH. MS(m/z, EI⁺):829.0

### EXAMPLE 6

### Synthesis of EX18

### Synthesis of Intermediate C

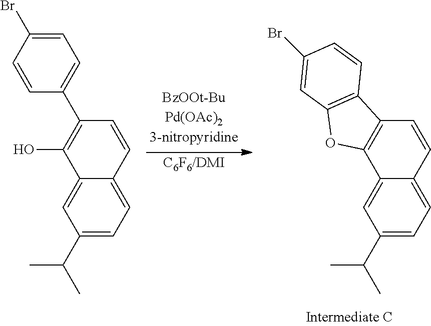 [SMILES: CC(C)c1ccc2ccc(-c3ccc(Br)cc3)c(O)c2c1 ; CC(C)c1ccc2ccc3c4ccc(Br)cc4oc3c2c1]

A mixture of 10 g (29.3 mole) of 2-(4-Bromophenyl)-7-isopropyl-naphthalen-1-ol, 0.66 g (2.93 mmole) of Pd(OAc)₂, 0.37 g (2.93 mmole) of 3-Nitropyridine, 11.4 g (58.6 mmole) of Tert-butyl peroxybenzoate, 100 ml of DMI, and 50 ml of C₆F₆ was placed under nitrogen, and then heated at 80° C. while stirring for 16 h. After the reaction finished, the mixture was allowed to cool to room temperature. The solution was extracted with dichloromethane and water. The organic layer was dried with anhydrous magnesium sulfate and then the solvent was evaporated under reduced pressure. The residue was purified by column chromatography on silica to give product (3.9 g, 39%) as a white solid.

### Synthesis of Intermediate D

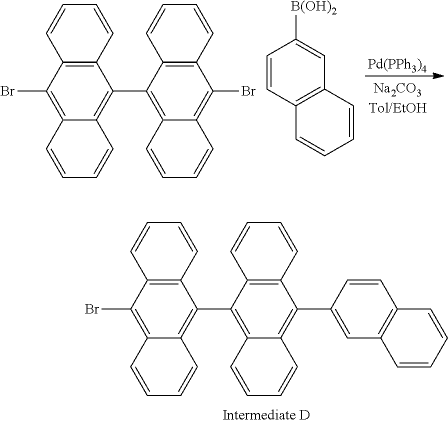

A mixture of 12.4 g (24.3 mmole) of 10,10′-Dibromo-9,9′-bianthracene, 5.0 g (29.1 mmole) of 2-Naphthylboronic acid, 0.6 g (0.5 mmole) of Pd(PPh₃)₄, 0.34 g (1.0 mmole) of 2-Dicyclophosphine-2′,6′-dimethoxybiphenyl, 3.9 g (36.5 mmole) of Na₂CO₃, 190 ml of Toluene and 60 ml of Ethanol, and 18 ml of H₂O was placed under nitrogen, and then heated at 80° C. while stirring for 16 h. After the reaction finished, the mixture was allowed to cool to room temperature. The solution was extracted with 100 ml of ethyl acetate (3 times) and then 300 ml of water. The organic layer was dried with anhydrous magnesium sulfate, and then the solvent was evaporated under reduced pressure. The residue was purified by column chromatography on silica to give product (10.0 g, 74%) as a white solid.

### Synthesis of Intermediate E

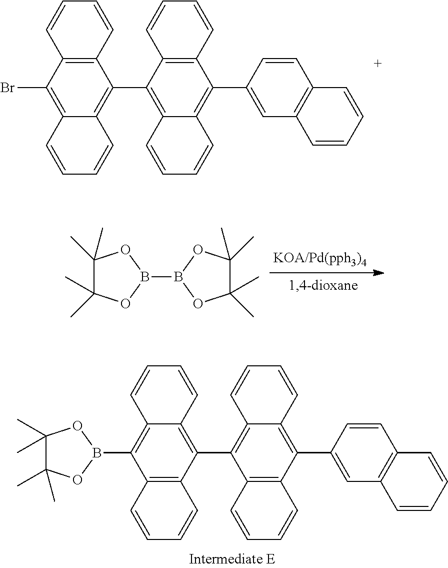

A mixture of 10.0 g (17.9 mmole) of Intermediate D, 5.4 g (21.5 mmol) of bis(pinacolato)diboron, 0.4 g (0.36 mmol) of Pd(PPh₃)₄, 5.3 g (53.7 mmol) of potassium acetate, and 300 ml of 1,4-dioxane was degassed and placed under nitrogen, and then heated at 90° C. for 16 h. After the reaction finished, the mixture was allowed to cool to room temperature. The solution was extracted with 150 ml of ethyl acetate (3 times) and then 300 ml of water. The organic layer was dried with anhydrous magnesium sulfate, and then the solvent was evaporated under reduced pressure. The residue was purified by column chromatography on silica to give product (8.4 g, 77%) as a off-white solid.

### Synthesis of EX18

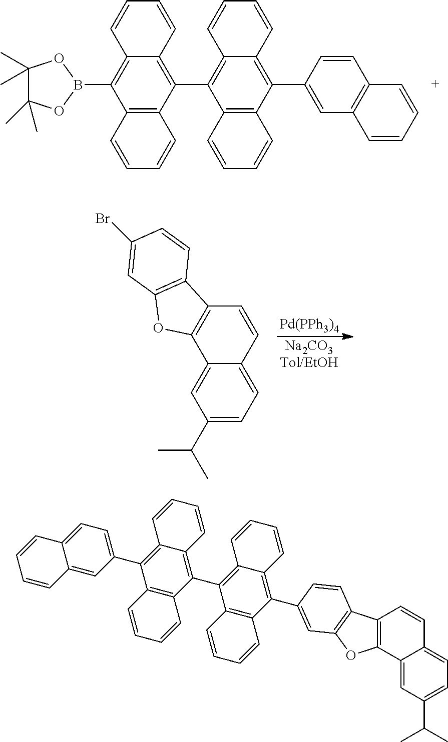

A mixture of 8.4 g (13.8 mmole) of Intermediate E, 3.9 g (11.5 mmole) of Intermediate C, 0.27 g (0.23 mmole) of Pd(PPh3)₄, 0.16 g (0.46 mmole) of 2-Dicyclophosphine-2′,6′-dimethoxybiphenyl, 1.8 g (17.3 mmole) of Na₂CO₃, 60 ml of Toluene and 20 ml of Ethanol, and 9 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (5.0 g, 59%) of off-white product, which was recrystallized from EtOH. MS(m/z, EI⁺):739.9

### EXAMPLE 7

### Synthesis of EX37

### Synthesis of Intermediate F

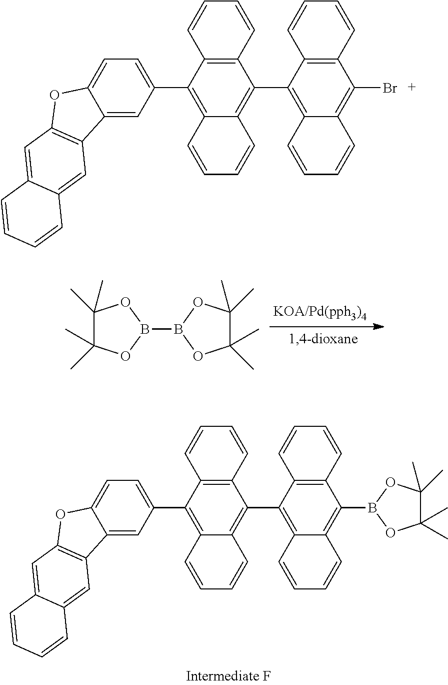

A mixture of 5.0 g (7.7 mmole) of Intermediate A, 2.3 g (9.2 mmol) of bis(pinacolato)diboron, 0.18 g (0.15 mmol) of Pd(PPh₃)₄, 2.3 g (23.1 mmol) of potassium acetate, and 150 ml of 1,4-dioxane was degassed and placed under nitrogen, and then heated at 90° C. for 16 h. After the reaction finished, the mixture was allowed to cool to room temperature. The solution was extracted with 100 ml of ethyl acetate (3 times) and then 300 ml of water. The organic layer was dried with anhydrous magnesium sulfate, and then the solvent was evaporated under reduced pressure. The residue was purified by column chromatography on silica to give product (3.6 g, 68%) as a off-white solid.

### Synthesis of EX37

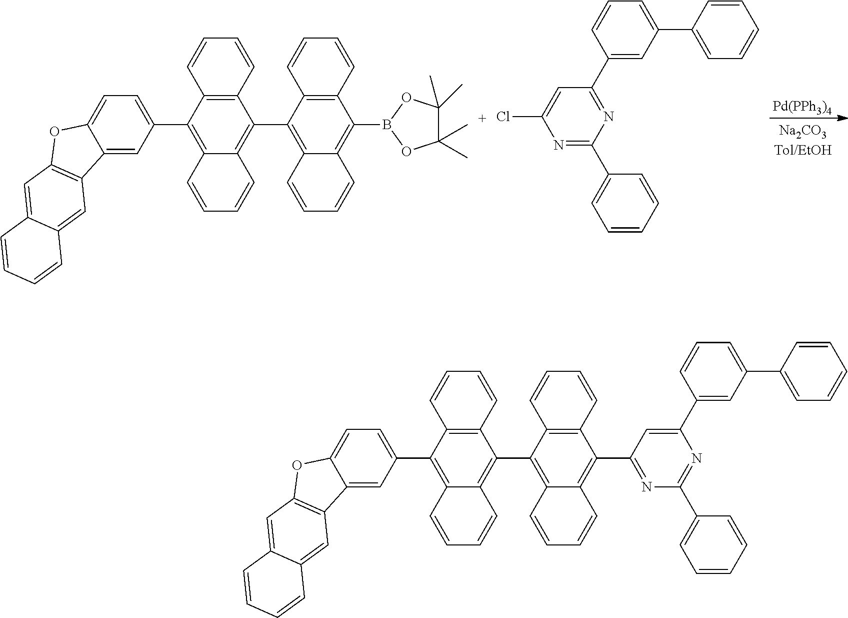

A mixture of 3.6 g (5.2 mmole) of Intermediate F, 2.1 g (6.2 mmole) of 4-([1,1′-biphenyl]-3-yl)-6-chloro-2-phenylpyrimidine, 0.12 g (0.1 mmole) of Pd(PPh₃)₄, 0.07 g (0.2 mmole) of 2-Dicyclophosphine-2′,6′-dimethoxybiphenyl, 0.8 g (7.8 mmole) of Na₂CO₃, 55 ml of Toluene and 18 ml of Ethanol, and 4 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (2.9 g, 63%) of off-white product, which was recrystallized from EtOH. MS(m/z, EI⁺):878.0

### EXAMPLE 8

### Synthesis of EX39

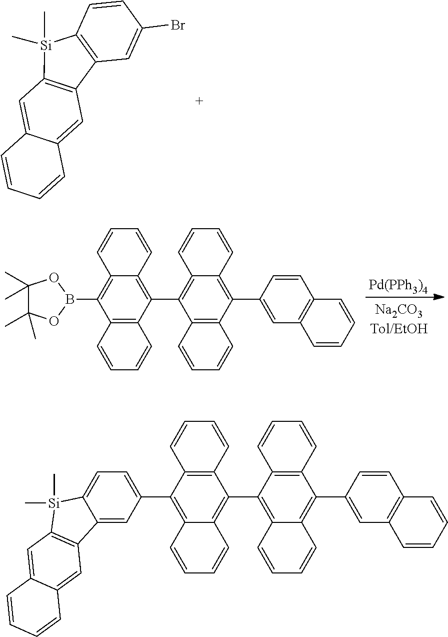 [SMILES: C[Si]1(C)c2ccc(Br)cc2-c2cc3ccccc3cc21 ; CC1(C)OB(c2c3ccccc3c(-c3c4ccccc4c(-c4ccc5ccccc5c4)c4ccccc34)c3ccccc23)OC1(C)C ; C[Si]1(C)c2ccc(-c3c4ccccc4c(-c4c5ccccc5c(-c5ccc6ccccc6c5)c5ccccc45)c4ccccc34)cc2-c2cc3ccccc3cc21 ; "Pd(PPh3)4"]

A mixture of 7.0 g (11.5 mmole) of Intermediate E, 3.3 g (9.6 mmole) of 2-bromo-5,5-dimethyl-5H-benzo[b]naphtho[2,3-d]silole, 0.22 g (0.2 mmole) of Pd(PPh₃)₄, 0.13 g (0.38 mmole) of 2-Dicyclophosphine-2′,6′-dimethoxybiphenyl, 1.5 g (14.4 mmole) of Na₂CO₃, 50 ml of Toluene and 17 ml of Ethanol, and 7 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (4.0 g, 57%) of off-white product, which was recrystallized from EtOH. MS(m/z, EI⁺):740.0

### EXAMPLE 9

### Synthesis of EX58

### Synthesis of Intermediate G

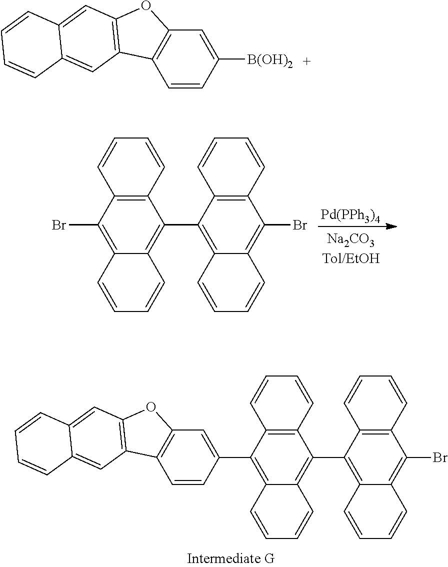 [SMILES: OB(O)c1ccc2c(c1)oc1cc3ccccc3cc12 ; Brc1c2ccccc2c(-c2c3ccccc3c(Br)c3ccccc23)c2ccccc12 ; Brc1c2ccccc2c(-c2c3ccccc3c(-c3ccc4c(c3)oc3cc5ccccc5cc34)c3ccccc23)c2ccccc12 ; "Pd(PPh3)4"]

A mixture of 8.2 g (16.0 mmole) of 10,10′-Dibromo-9,9′-bianthracene, 5.0 g (19.1 mmole) of Naphtho[2,3-b]benzofuran-3-ylboronic acid, 0.4 g (0.3 mmole) of Pd(PPh₃)₄, 0.2 g (0.6 mmole) of 2-Dicyclophosphine-2′,6′-dimethoxybiphenyl, 2.5 g (24.0 mmole) of Na₂CO₃, 120 ml of Toluene and 40 ml of Ethanol, and 12 ml of H₂O was placed under nitrogen, and then heated at 80° C. while stirring for 16 h. After the reaction finished, the mixture was allowed to cool to room temperature. The solution was extracted with 100 ml of ethyl acetate (3 times) and then 300 ml of water. The organic layer was dried with anhydrous magnesium sulfate, and then the solvent was evaporated under reduced pressure. The residue was purified by column chromatography on silica to give product (7.3 g, 70%) as a white solid.

### Synthesis of EX58

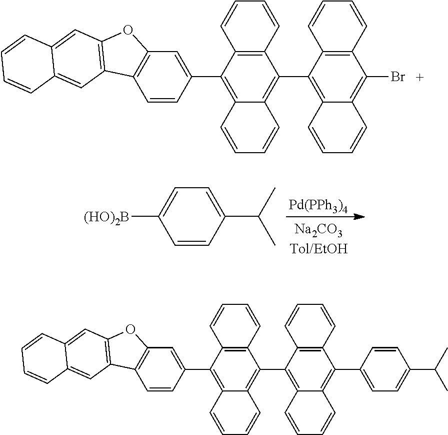

A mixture of 7.3 g (11.2 mmole) of Intermediate G, 2.2 g (13.5 mmole) of 4-Isopropylphenylboronic acid, 0.26 g (0.22 mmole) of Pd(PPh₃)₄, 0.16 g (0.45 mmole) of 2-Dicyclophosphine-2′,6′-dimethoxybiphenyl, 1.8 g (16.8 mmole) of Na₂CO₃, 110 ml of Toluene and 37 ml of Ethanol, and 9 ml of H₂O was placed under nitrogen, and then heated at 80° C. while stirring for 16 h. After the reaction finished, the mixture was allowed to cool to room temperature. Then 700 ml of MeOH was added while stirring and the precipitated product was filtered off with suction to give (4.9 g, 63%) of off-white product, which was recrystallized from EtOH. MS(m/z, EI⁺):689.8

### EXAMPLE 10

### Synthesis of EX105

### Synthesis of Intermediate H

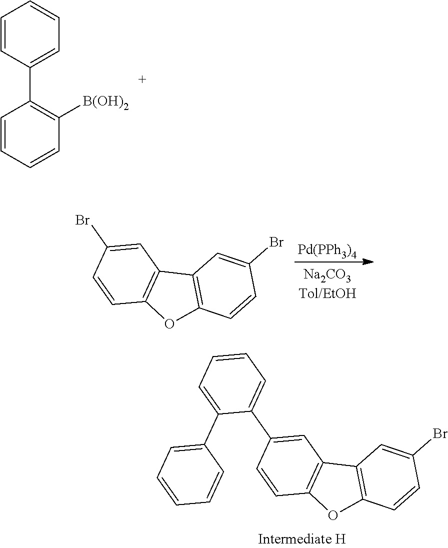

A mixture of 32.6 g (100 mmol) of 2,8-dibromodibenzo[b,d]furan, 21.8 g (110 mmol) of biphenyl-2-ylboronic acid, 2.31 g (2 mmol) of Pd(PPh₃)₄, 75 ml of 2M Na₂CO₃, 150 ml of EtOH and 300 ml toluene was degassed and placed under nitrogen, and then heated at 100° C. for 12 h. After finishing the reaction, the mixture was allowed to cool to room temperature. The organic layer was extracted with ethyl acetate and water, dried with anhydrous magnesium sulfate, the solvent was removed and the residue was purified by column chromatography on silica to give product (25.1 g, 63%) as a white solid.

### Synthesis of Intermediate I

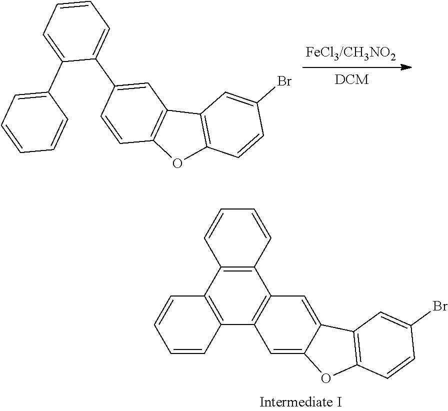 [SMILES: Brc1ccc2oc3ccc(-c4ccccc4-c4ccccc4)cc3c2c1 ; Brc1ccc2oc3cc4c5ccccc5c5ccccc5c4cc3c2c1]

In a 3000 ml three-necked flask that had been degassed and filled with nitrogen, 25.1 g (63 mmol) of Intermediate H was dissolved in anhydrous dichloromethane (1500 ml), 102.2 g (630 mmol) iron(III) chloride was then added, and the mixture was stirred one hour. Methanol 500 ml were added to the mixture and the organic layer was separated and the solvent removed in vacuo. The residue was purified by column chromatography on silica to give product (5.7 g, 23%) as a yellow solid.

### Synthesis of EX110

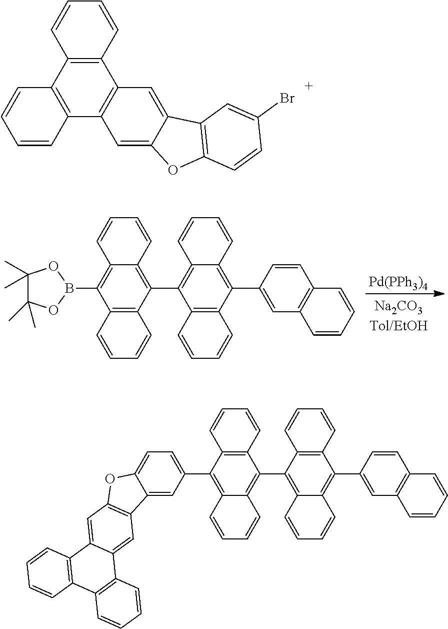 [SMILES: Brc1ccc2oc3cc4c5ccccc5c5ccccc5c4cc3c2c1 ; CC1(C)OB(c2c3ccccc3c(-c3c4ccccc4c(-c4ccc5ccccc5c4)c4ccccc34)c3ccccc23)OC1(C)C ; c1ccc2cc(-c3c4ccccc4c(-c4c5ccccc5c(-c5ccc6oc7cc8c9ccccc9c9ccccc9c8cc7c6c5)c5ccccc45)c4ccccc34)ccc2c1 ; "Pd(PPh3)4"]

A mixture of 8.4 g (13.8 mmole) of Intermediate E, 4.6 g (11.5 mmole) of Intermediate I, 0.27 g (0.23 mmole) of Pd(PPh₃)₄, 0.16 g (0.46 mmole) of 2-Dicyclophosphine-2′,6′-dimethoxybiphenyl, 1.8 g (17.3 mmole) of Na₂CO₃, 60 ml of Toluene and 20 ml of Ethanol, and 9 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (5.0 g, 55%) of yellow product. MS(m/z , EI⁺):797.9

### EXAMPLE 11

### Synthesis of EX114

### Synthesis of Intermediate J

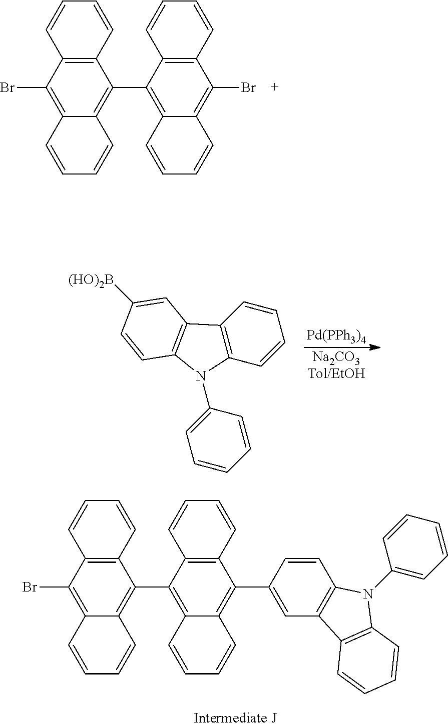

A mixture of 12.4 g (24.3 mmole) of 10,10′-Dibromo-9,9′-bianthracene, 8.3 g (29.1 mmole) of 9-Phenyl-9H-carbazol-3-ylboronic acid, 0.6 g (0.5 mmole) of Pd(PPh₃)₄, 0.34 g (1.0 mmole) of 2-Dicyclophosphine-2′,6′-dimethoxybiphenyl, 3.9 g (36.5 mmole) of Na₂CO₃, 190 ml of Toluene and 60 ml of Ethanol, and 18 ml of H₂O was placed under nitrogen, and then heated at 80° C. while stirring for 16 h. After the reaction finished, the mixture was allowed to cool to room temperature. The solution was extracted with 100 ml of ethyl acetate (3 times) and then 300 ml of water. The organic layer was dried with anhydrous magnesium sulfate, and then the solvent was evaporated under reduced pressure. The residue was purified by column chromatography on silica to give product (9.7 g, 59%) as a white solid.

### Synthesis of Intermediate K

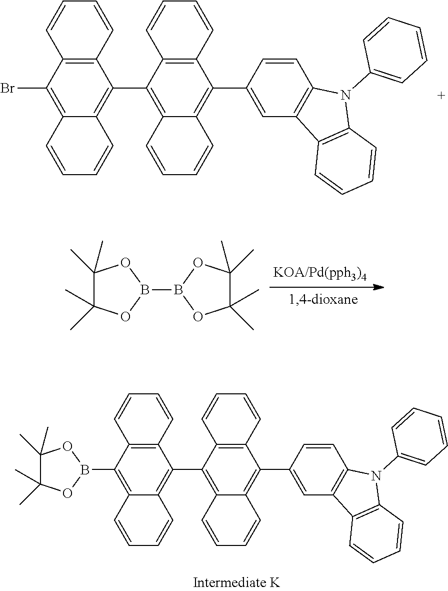

A mixture of 9.7 g (14.4 mmole) of Intermediate J, 4.4 g (17.3 mmol) of bis(pinacolato)diboron, 0.3 g (0.28 mmol) of Pd(PPh₃)₄, 4.3 g (43.2 mmol) of potassium acetate, and 290 ml of 1,4-dioxane was degassed and placed under nitrogen, and then heated at 90° C. for 16 h. After the reaction finished, the mixture was allowed to cool to room temperature. The solution was extracted with 150 ml of ethyl acetate (3 times) and then 300 ml of water. The organic layer was dried with anhydrous magnesium sulfate, and then the solvent was evaporated under reduced pressure. The residue was purified by column chromatography on silica to give product (7.5 g, 72%) as a off-white solid.

### Synthesis of EX114

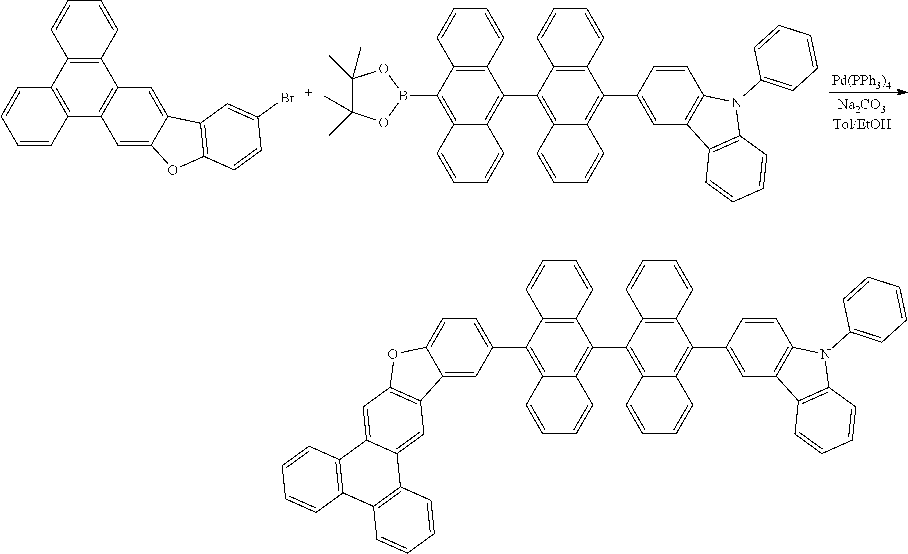

A mixture of 3.4 g (8.7 mmole) of Intermediate I, 7.5 g (10.4 mmole) of Intermediate K, 0.21 g (0.17 mmole) of Pd(PPh₃)₄, 0.12 g (0.35 mmole) of 2-Dicyclophosphine-2′,6′-dimethoxybiphenyl, 1.4 g (13.1 mmole) of Na₂CO₃, 50 ml of Toluene and 17 ml of Ethanol, and 7 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (4.0 g, 51%) of yellow product. MS(m/z , EI⁺):913.1

### EXAMPLE 12

### Synthesis of EX130

### Synthesis of Intermediate L

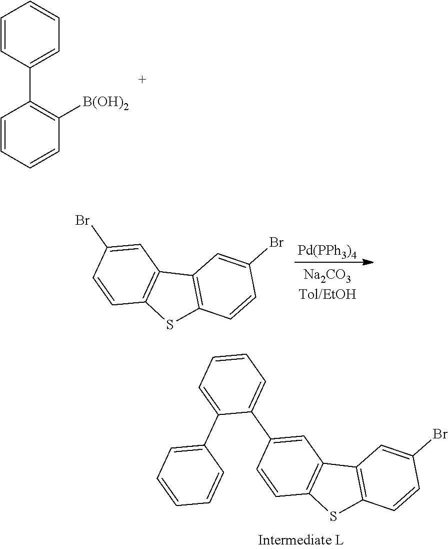

A mixture of 34.2 g (100 mmol) of 2,8-Dibromodibenzo[b,d]thiophene, 21.8 g (110 mmol) of biphenyl-2-ylboronic acid, 2.31 g (2 mmol) of Pd(PPh₃)₄, 75 ml of 2M Na₂CO₃, 150 ml of EtOH and 300 ml toluene was degassed and placed under nitrogen, and then heated at 100° C. for 12 h. After finishing the reaction, the mixture was allowed to cool to room temperature. The organic layer was extracted with ethyl acetate and water, dried with anhydrous magnesium sulfate, the solvent was removed and the residue was purified by column chromatography on silica to give product (25.3 g, 61%) as a white solid.

### Synthesis of Intermediate M

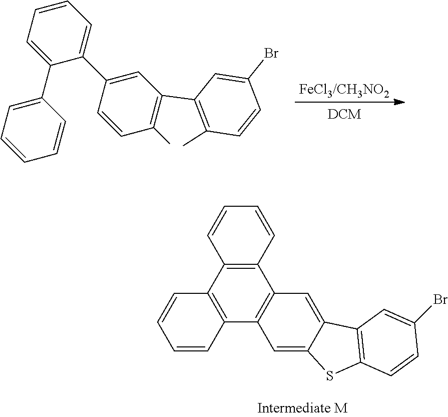 [SMILES: Cc1ccc(Br)cc1-c1cc(-c2ccccc2-c2ccccc2)ccc1C ; Brc1ccc2sc3cc4c5ccccc5c5ccccc5c4cc3c2c1]

In a 3000 ml three-necked flask that had been degassed and filled with nitrogen, 25.3 g (61 mmol) of Intermediate L was dissolved in anhydrous dichloromethane (1500 ml), 98.9g (610 mmol) iron(III) chloride was then added, and the mixture was stirred one hour. Methanol 500 ml were added to the mixture and the organic layer was separated and the solvent removed in vacuo. The residue was purified by column chromatography on silica to give product (6.8 g, 27%) as a yellow solid.

### Synthesis of EX130

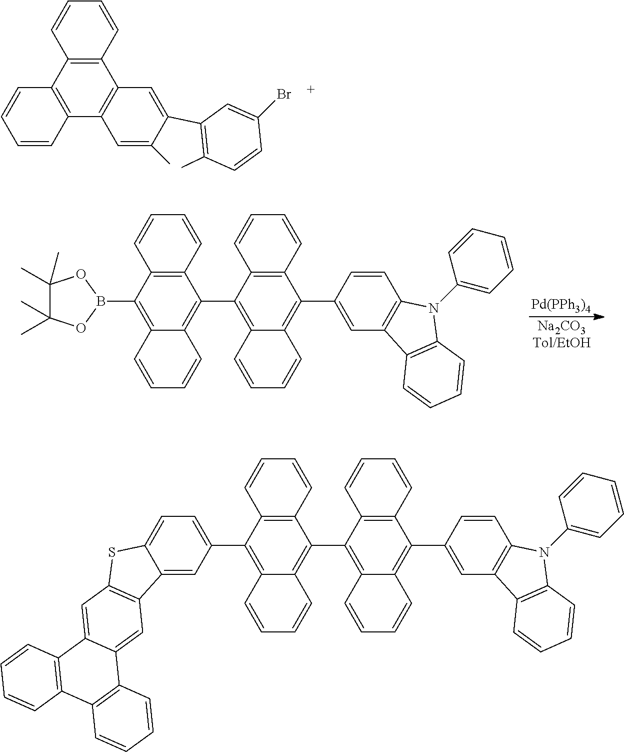 [SMILES: Cc1ccc(Br)cc1-c1cc2c3ccccc3c3ccccc3c2cc1C ; CC1(C)OB(c2c3ccccc3c(-c3c4ccccc4c(-c4ccc5c(c4)c4ccccc4n5-c4ccccc4)c4ccccc34)c3ccccc23)OC1(C)C ; "Na2CO3Tol/EtOH" ; c1ccc(-n2c3ccccc3c3cc(-c4c5ccccc5c(-c5c6ccccc6c(-c6ccc7sc8cc9c%10ccccc%10c%10ccccc%10c9cc8c7c6)c6ccccc56)c5ccccc45)ccc32)cc1 ; "Pd(PPh3)4"]

A mixture of 3.6 g (8.7 mmole) of Intermediate M, 7.5 g (10.4 mmole) of Intermediate K, 0.21 g (0.17 mmole) of Pd(PPh₃)₄, 0.12 g (0.35 mmole) of 2-Dicyclophosphine-2′,6′-dimethoxybiphenyl, 1.4 g (13.1 mmole) of Na₂CO₃, 50 ml of Toluene and 17 ml of Ethanol, and 7 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (4.2 g, 52%) of yellow product. MS(m/z , EI⁺):929.1

### EXAMPLE 13

### Synthesis of EX146

### Synthesis of Intermediate N

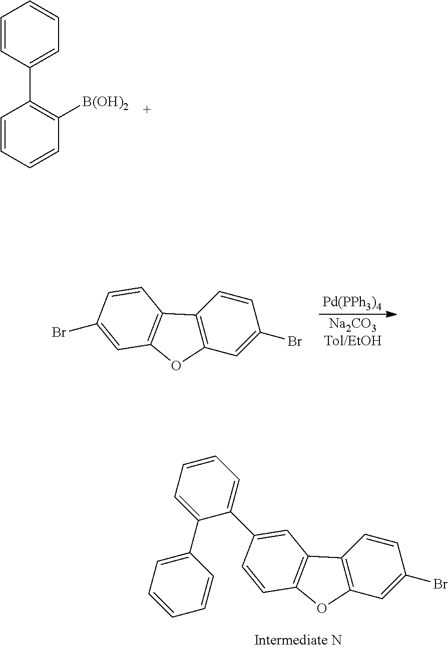 [SMILES: OB(O)c1ccccc1-c1ccccc1 ; Brc1ccc2c(c1)oc1cc(Br)ccc12 ; "Na2CO3Tol/EtOH" ; Brc1ccc2c(c1)oc1ccc(-c3ccccc3-c3ccccc3)cc12 ; "Pd(PPh3)4"]

A mixture of 32.6 g (100 mmol) of 3,7-dibromodibenzo[b,d]furan, 21.8 g (110 mmol) of biphenyl-2-ylboronic acid, 2.31 g (2 mmol) of Pd(PPh₃)₄, 75 ml of 2M Na₂CO₃, 150 ml of EtOH and 300 ml toluene was degassed and placed under nitrogen, and then heated at 100° C. for 12 h. After finishing the reaction, the mixture was allowed to cool to room temperature. The organic layer was extracted with ethyl acetate and water, dried with anhydrous magnesium sulfate, the solvent was removed and the residue was purified by column chromatography on silica to give product (24.3 g, 61%) as a white solid.

### Synthesis of Intermediate O

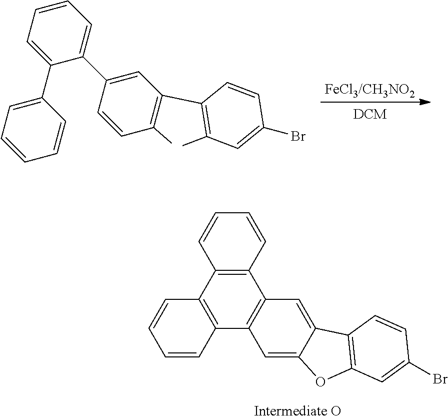 [SMILES: Cc1cc(Br)ccc1-c1cc(-c2ccccc2-c2ccccc2)ccc1C ; Brc1ccc2c(c1)oc1cc3c4ccccc4c4ccccc4c3cc12]

In a 3000 ml three-necked flask that had been degassed and filled with nitrogen, 24.3 g (61 mmol) of Intermediate N was dissolved in anhydrous dichloromethane (1500 ml), 98.9 g (610 mmol) iron(III) chloride was then added, and the mixture was stirred one hour. Methanol 500 ml were added to the mixture and the organic layer was separated and the solvent removed in vacuo. The residue was purified by column chromatography on silica to give product (7.0 g, 29%) as a yellow solid.

### Synthesis of EX146

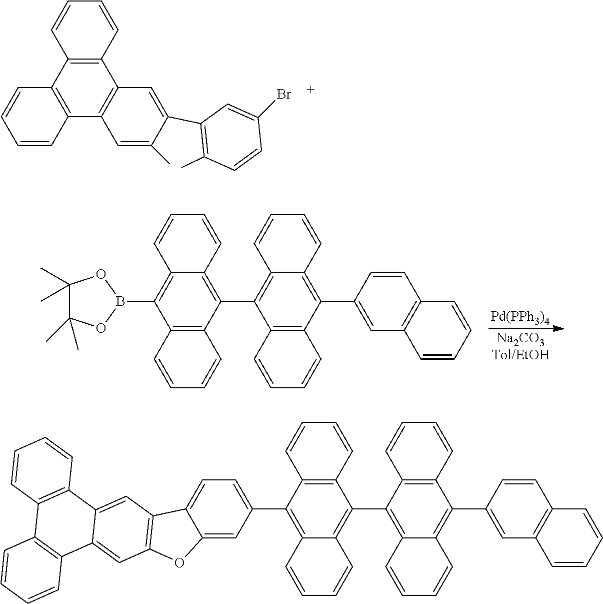

A mixture of 8.4 g (13.8 mmole) of Intermediate E, 4.6 g (11.5 mmole) of Intermediate O, 0.27 g (0.23 mmole) of Pd(PPh₃)₄, 0.16 g (0.46 mmole) of 2-Dicyclophosphine-2′,6′-dimethoxybiphenyl, 1.8 g (17.3 mmole) of Na₂CO₃, 60 ml of Toluene and 20 ml of Ethanol, and 9 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (5.2 g, 57%) of yellow product. MS(m/z , EI⁺):797.9

### EXAMPLE 14

### Synthesis of EX157

### Synthesis of Intermediate P

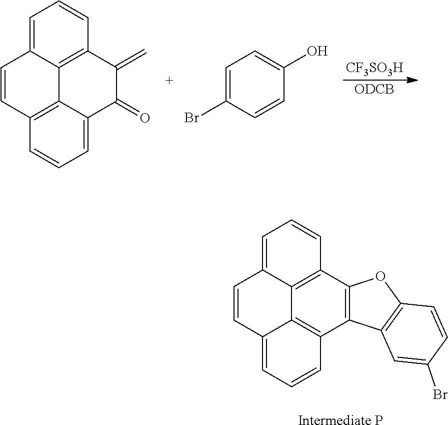

A mixture of 12 g (51.7 mmol) of Pyrene-4,5-diketone, 7.7 g (51.7 mmol) of Trifluoromethanesulfonic acid, 8.9 g (51.7 mmol) of 4-Bromophenol, 200 ml of 1,2-Dichlorobenzene was degassed and placed under nitrogen, and then heated at 190° C. for 24 h. After finishing the reaction, the solvent was removed and the residue was purified by column chromatography on silica to give product (3.3 g, 17%) as a light-green solid.

### Synthesis of EX157

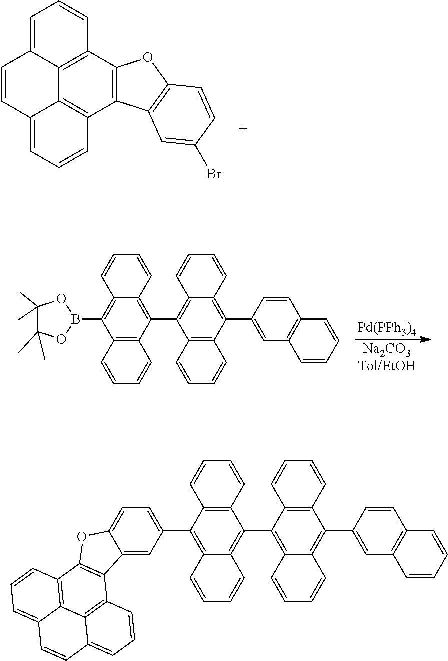 [SMILES: Brc1ccc2oc3c4cccc5ccc6cccc(c3c2c1)c6c54 ; CC1(C)OB(c2c3ccccc3c(-c3c4ccccc4c(-c4ccc5ccccc5c4)c4ccccc34)c3ccccc23)OC1(C)C ; "Na2CO3Tol/EtOH" ; c1ccc2cc(-c3c4ccccc4c(-c4c5ccccc5c(-c5ccc6oc7c8cccc9ccc%10cccc(c7c6c5)c%10c98)c5ccccc45)c4ccccc34)ccc2c1 ; "Pd(PPh3)4"]

A mixture of 6.5 g (10.7 mmole) of Intermediate E, 3.3 g (8.9 mmole) of Intermediate P, 0.20 g (0.18 mmole) of Pd(PPh₃)₄, 0.12 g (0.36 mmole) of 2-Dicyclophosphine-2′,6′-dimethoxybiphenyl, 1.4 g (13.3 mmole) of Na₂CO₃, 50 ml of Toluene and 17 ml of Ethanol, and 7 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (3.8 g, 56%) of yellow product. MS(m/z , EI⁺):771.9

### EXAMPLE 15

### Synthesis of EX171

### Synthesis of Intermediate Q

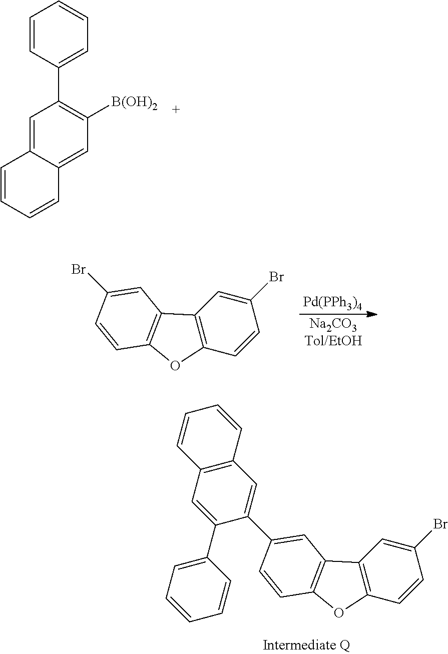 [SMILES: OB(O)c1cc2ccccc2cc1-c1ccccc1 ; Brc1ccc2oc3ccc(Br)cc3c2c1 ; "Na2CO3Tol/EtOH" ; Brc1ccc2oc3ccc(-c4cc5ccccc5cc4-c4ccccc4)cc3c2c1 ; "Pd(PPh3)4"]

A mixture of 32.6 g (100 mmol) of 2,8-dibromodibenzo[b,d]furan, 27.3 g (110 mmol) of (3-phenylnaphthalen-2-yl)boronic acid, 2.31 g (2 mmol) of Pd(PPh₃)₄, 75 ml of 2M Na₂CO₃, 150 ml of EtOH and 300 ml toluene was degassed and placed under nitrogen, and then heated at 100° C. for 12 h. After finishing the reaction, the mixture was allowed to cool to room temperature. The organic layer was extracted with ethyl acetate and water, dried with anhydrous magnesium sulfate, the solvent was removed and the residue was purified by column chromatography on silica to give product (23.8 g, 53%) as a white solid.

### Synthesis of Intermediate R

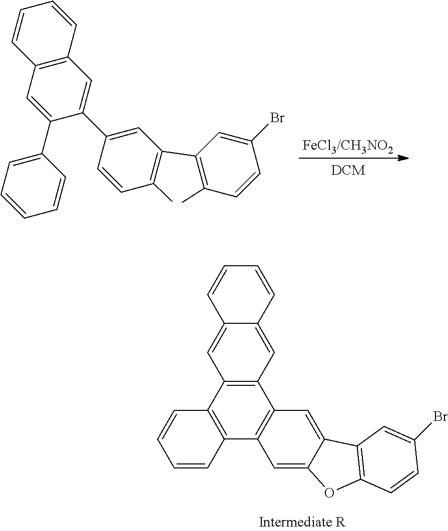 [SMILES: Cc1ccc(Br)cc1-c1cc(-c2cc3ccccc3cc2-c2ccccc2)ccc1C ; Brc1ccc2oc3cc4c5ccccc5c5cc6ccccc6cc5c4cc3c2c1]

In a 3000 ml three-necked flask that had been degassed and filled with nitrogen, 23.8 g (53 mmol) of Intermediate Q was dissolved in anhydrous dichloromethane (1500 ml), 86.0 g (530 mmol) iron(III) chloride was then added, and the mixture was stirred one hour. Methanol 500 ml were added to the mixture and the organic layer was separated and the solvent removed in vacuo. The residue was purified by column chromatography on silica to give product (5.0 g, 21%) as a yellow solid.

### Synthesis of EX171

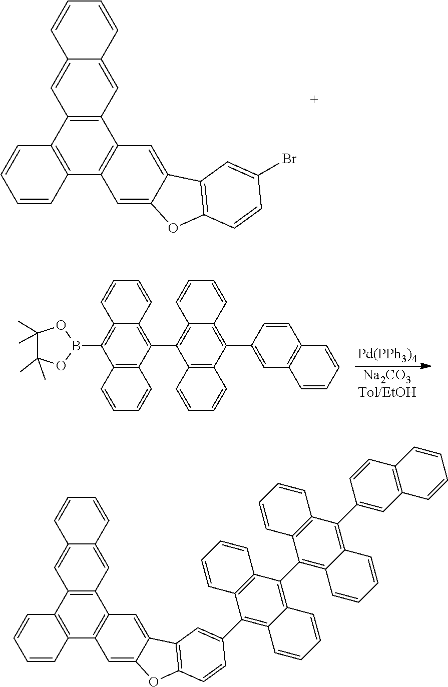 [SMILES: Brc1ccc2oc3cc4c5ccccc5c5cc6ccccc6cc5c4cc3c2c1 ; CC1(C)OB(c2c3ccccc3c(-c3c4ccccc4c(-c4ccc5ccccc5c4)c4ccccc34)c3ccccc23)OC1(C)C ; "Na2CO3Tol/EtOH" ; c1ccc2cc(-c3c4ccccc4c(-c4c5ccccc5c(-c5ccc6oc7cc8c9ccccc9c9cc%10ccccc%10cc9c8cc7c6c5)c5ccccc45)c4ccccc34)ccc2c1 ; "Pd(PPh3)4"]

A mixture of 8.4 g (13.8 mmole) of Intermediate E, 5.0 g (11.2 mmole) of Intermediate R, 0.27 g (0.23 mmole) of Pd(PPh₃)₄, 0.16 g (0.46 mmole) of 2-Dicyclophosphine-2′,6′-dimethoxybiphenyl, 1.8 g (17.3 mmole) of Na₂CO₃, 60 ml of Toluene and 20 ml of Ethanol, and 9 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (4.4 g, 47%) of yellow product. MS(m/z , EI⁺):848.0

### EXAMPLE 16

### Synthesis of EX173

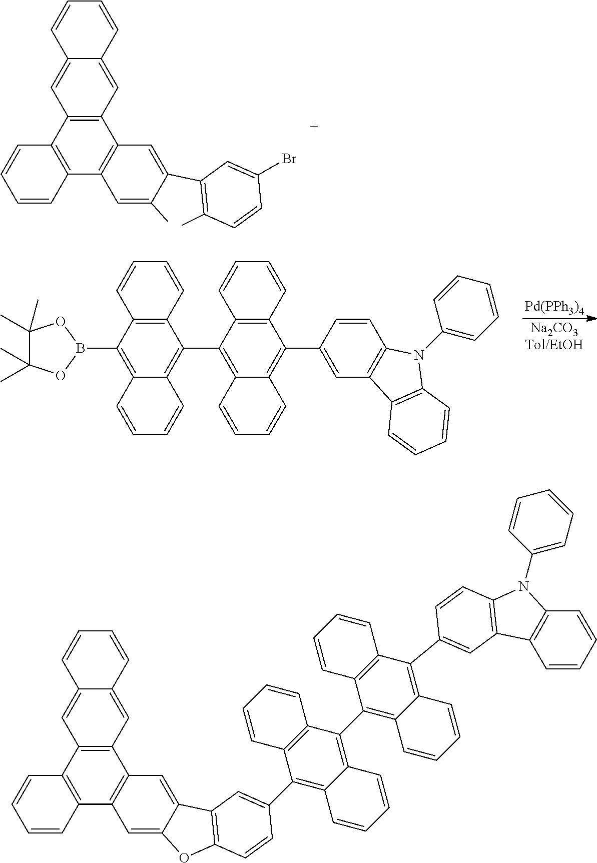

A mixture of 3.9 g (8.7 mmole) of Intermediate R, 7.5 g (10.4 mmole) of Intermediate K, 0.21 g (0.17 mmole) of Pd(PPh₃)₄, 0.12 g (0.35 mmole) of 2-Dicyclophosphine-2′,6′-dimethoxybiphenyl, 1.4 g (13.1 mmole) of Na₂CO₃, 50 ml of Toluene and 17 ml of Ethanol, and 7 ml of H₂O was placed under nitrogen, and then heated at 80° C. while stirring for 16 h. After the reaction finished, the mixture was allowed to cool to room temperature. Then 500 ml of MeOH was added while stirring and the precipitated product was filtered off with suction to give (3.4 g, 41%) of yellow product. MS(m/z , EI⁺):963.1

### General Method of Producing Organic El Device

ITO-coated glasses with 9-12 ohm/square in resistance and 120-160 nm in thickness are provided (hereinafter ITO substrate) and cleaned in a number of cleaning steps in an ultrasonic bath (e.g. detergent, deionized water). Before vapor deposition of the organic layers, cleaned ITO substrates are further treated by UV and ozone. All pre-treatment processes for ITO substrate are under clean room (class 100).

The organic layers are applied onto the ITO substrate in order by vapor deposition in a high-vacuum unit (10⁻⁷ Torr), such as: resistively heated quartz boats. The thickness of the respective layer and the vapor deposition rate (0.1˜0.3 nm/sec) are precisely monitored or set with the aid of a quartz-crystal monitor. It is also possible, as described above, for individual layers to consist of more than one compound, i.e. in general a host material doped with a dopant material. This is successfully achieved by co-vaporization from two or more sources, which means the organic compounds of the present invention are thermally stable.

Dipyrazino[2,3-f:2,3-]quinoxaline-2,3,6,7,10,11-hexacarbonitrile(HAT-CN) is used as hole injection layer in this organic EL device, N,N-Bis(naphthalene-1-yl)-N,N-bis(phenyl)-benzidine(NPB) is most widely used as the hole transporting layer; 10,10-dimethyl-13-(3-(pyren-1-yl)phenyl)-10H-indeno[2,1-b]triphenylene(H1) and 10,10-dimethyl-12-(10-(4-(naphthalene-1-yl)-phenyl)anthracen-9-yl)-10H-indeno[2,1-b]triphenylene(H2) are used as emitting hosts in organic EL device. D1 is used as blue guest, D2 is used as green guest for comparison; HB3(see the following chemical structure) are used as hole blocking material(HBM) and 2-(naphthalen-1-yl)-9-(4-(1-(4-(10-(naphthalene-2-yl) anthracen-9-yl)phenyl)-1H-benzo[d]imidazol-2-yl)phenyl)-1,10-phenanthroline(ET2) is used as electron transporting material to co-deposit with 8-hydroxyquinolato-lithium(LiQ) in organic EL device. The prior art of OLED materials for producing standard organic EL device control and comparable material in this invention shown its chemical structure as follows:

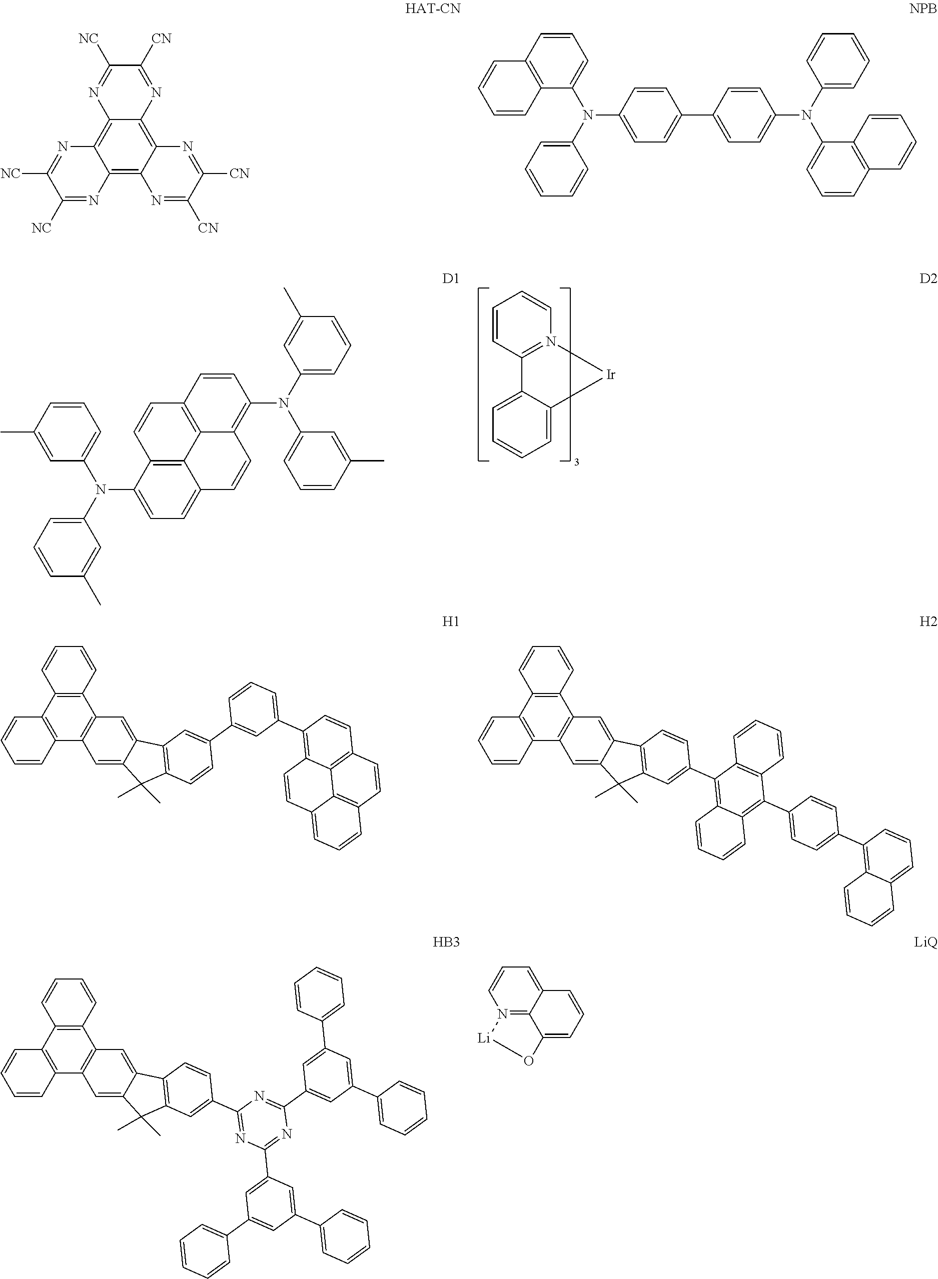

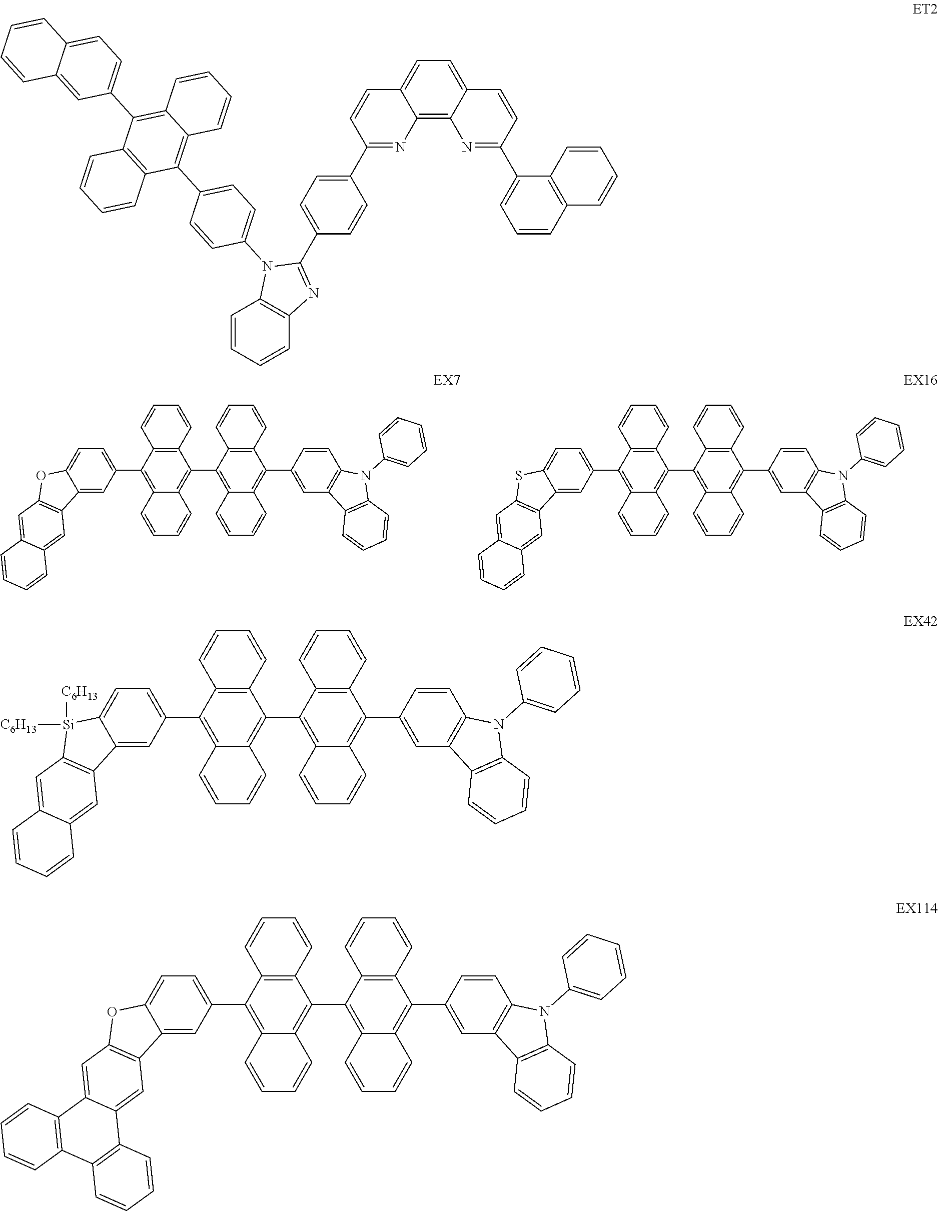

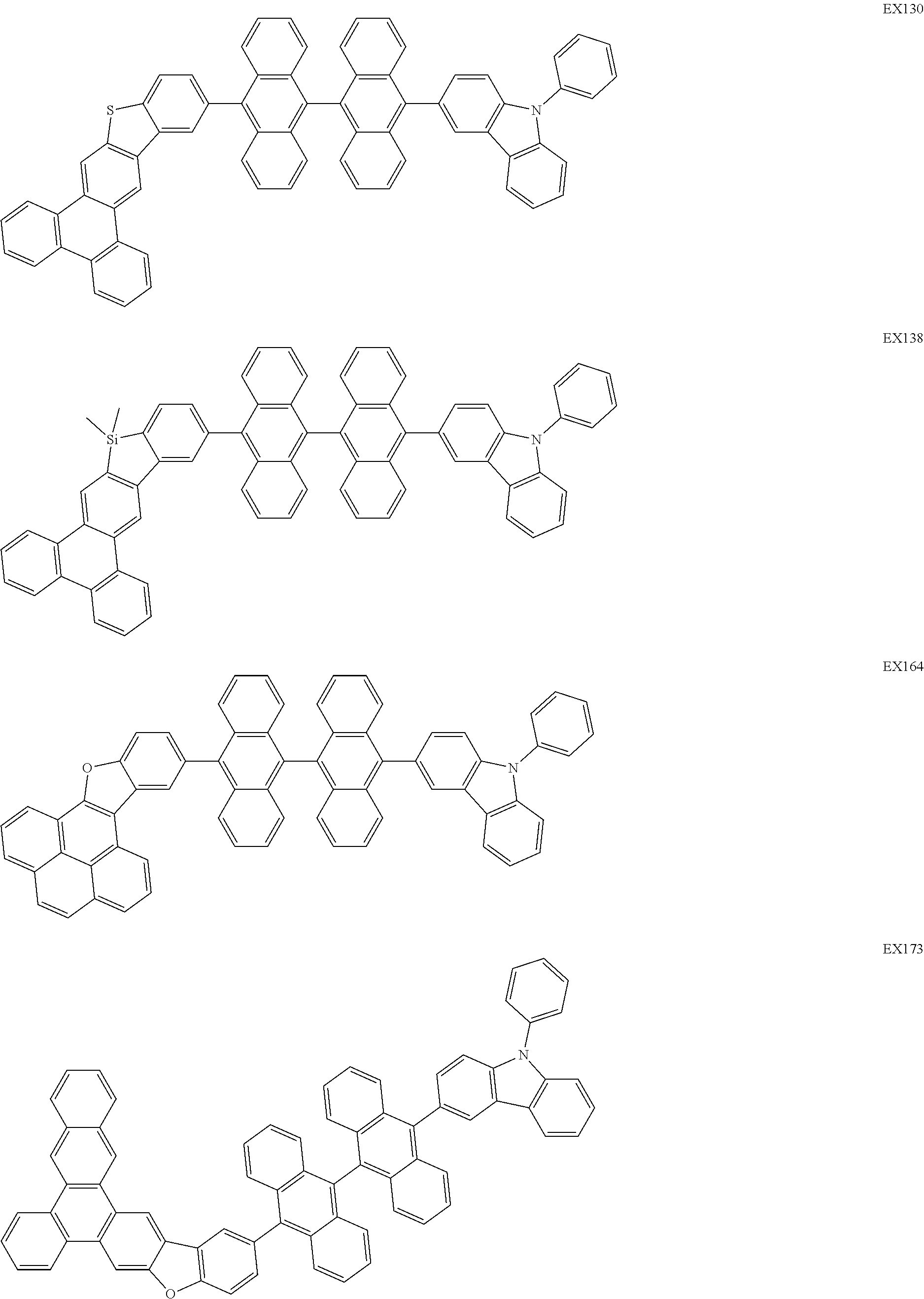

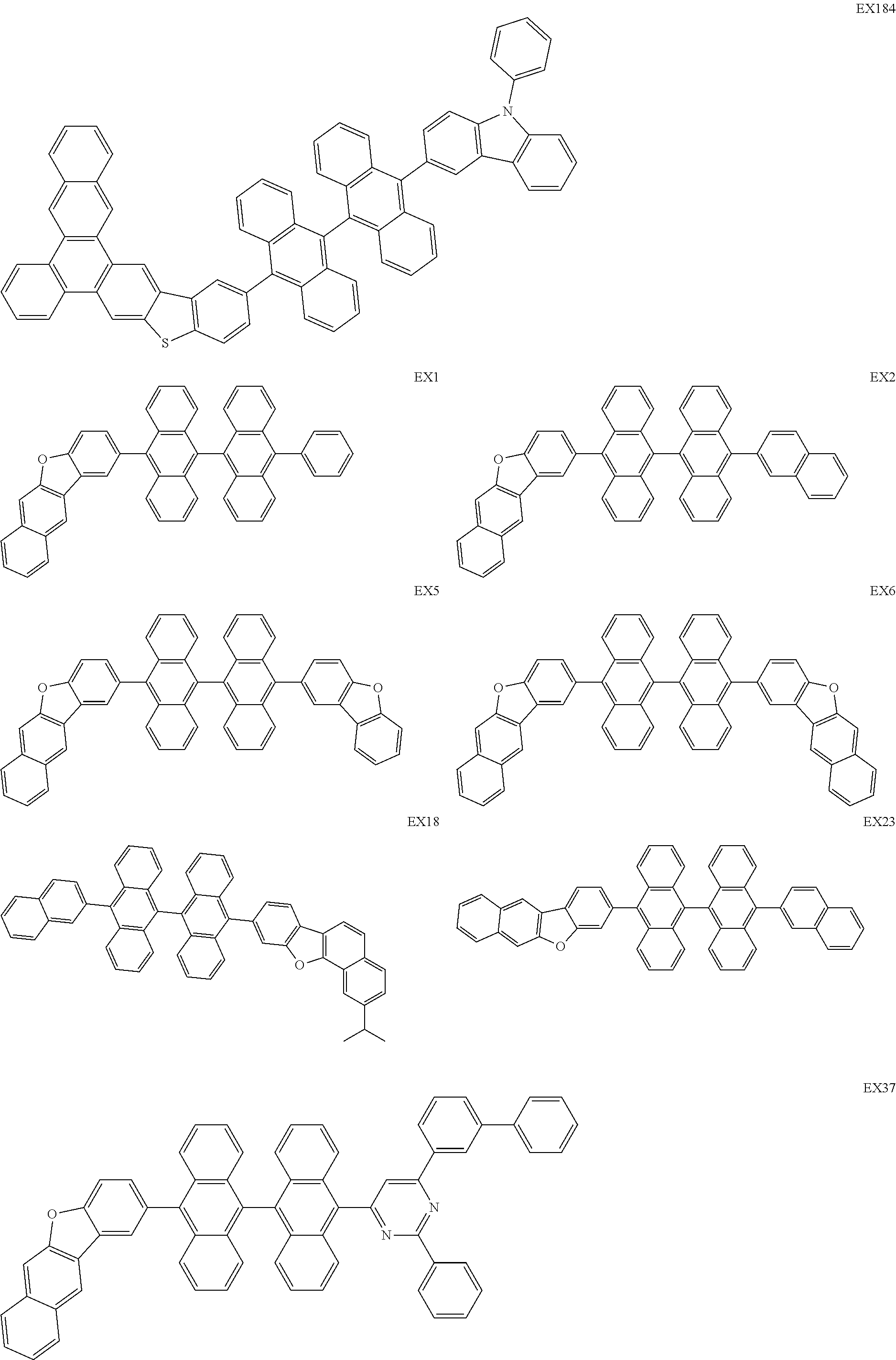

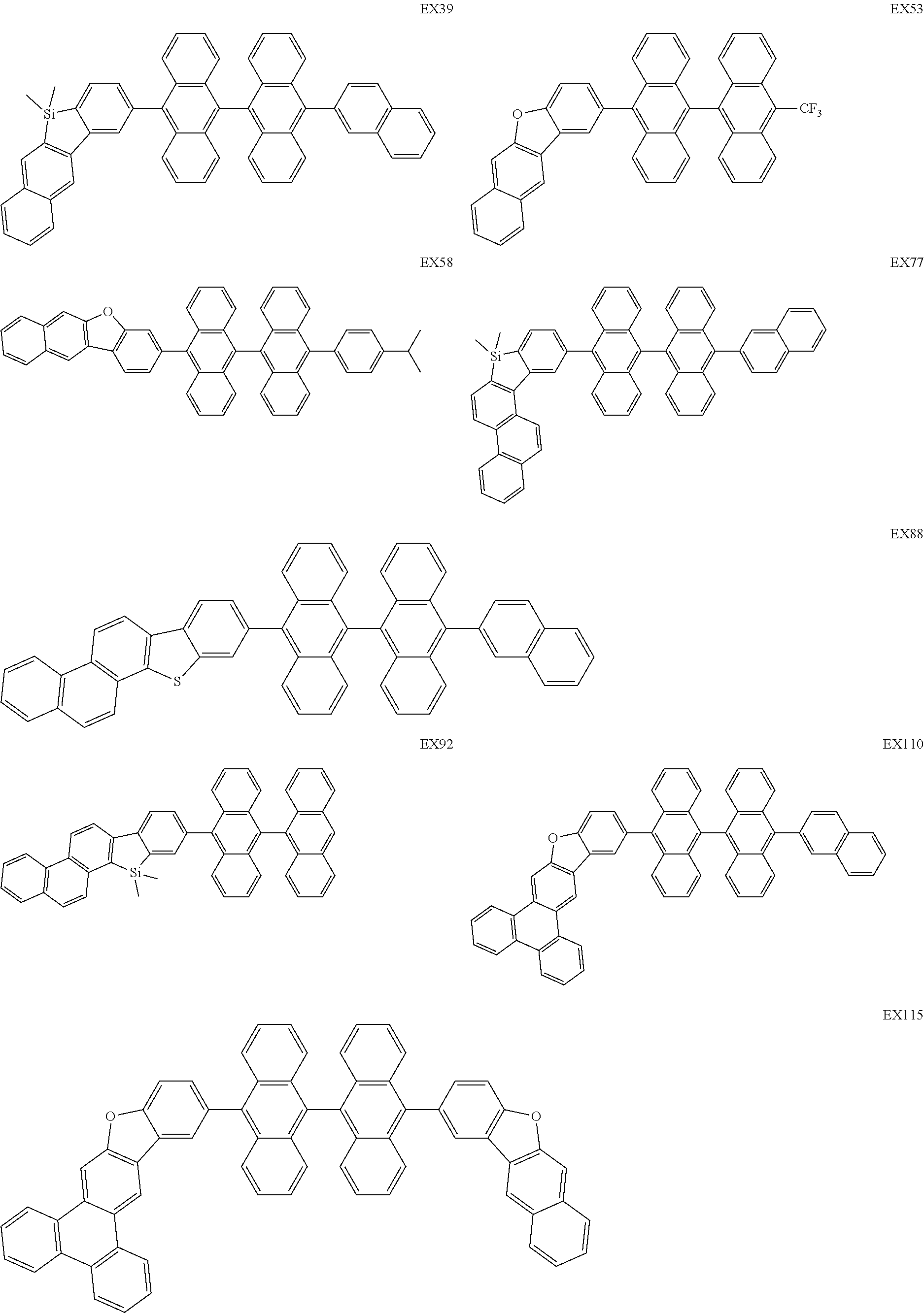

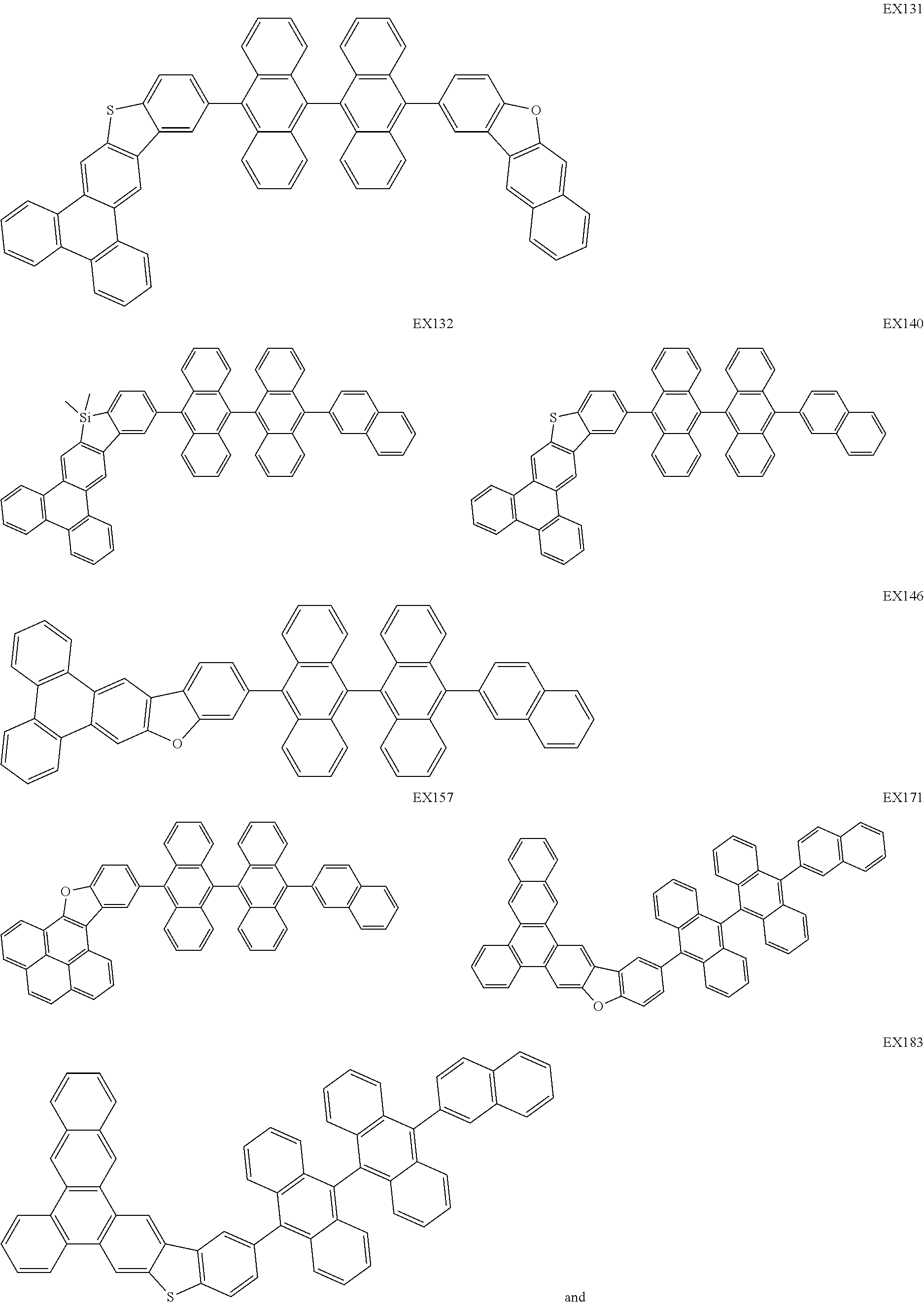

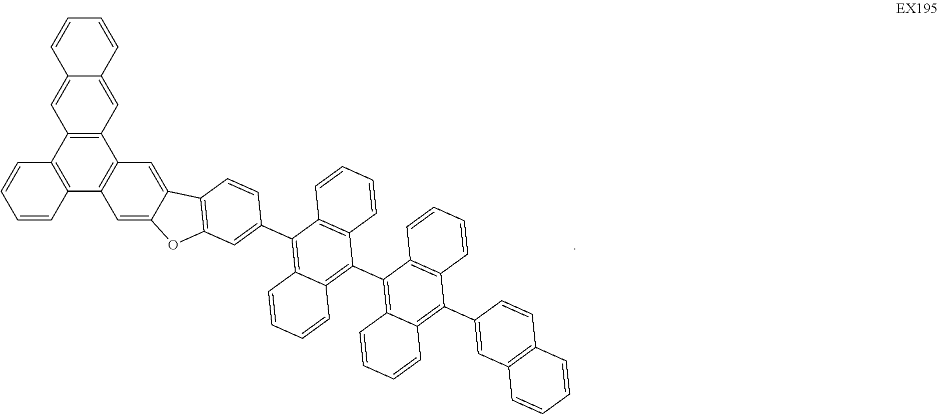 [SMILES: c1ccc2cc(-c3c4ccccc4c(-c4c5ccccc5c(-c5ccc6c(c5)oc5cc7c8ccccc8c8cc9ccccc9cc8c7cc56)c5ccccc45)c4ccccc34)ccc2c1]

A typical organic EL device consists of low work function metals, such as Al, Mg, Ca, Li and K, as the cathode, and the low work function metals can help electrons injecting the electron transporting layer from cathode. In addition, for reducing the electron injection barrier and improving the organic EL device performance, a thin-film electron injecting layer is introduced between the cathode and the electron transporting layer. The materials of electron injecting layer are metal halide or metal oxide with low work function, such as: LiF, LiQ, MgO, or Li₂O. On the other hand, after the organic EL device fabrication, EL spectra and CIE coordination are measured by using a PR650 spectra scan spectrometer. Furthermore, the current/voltage, luminescence/voltage and yield/voltage characteristics are taken with a Keithley 2400 programmable voltage-current source. The above-mentioned apparatuses are operated at room temperature (about 25° C.) and under atmospheric pressure.

### EXAMPLE 17

Using a procedure analogous to the above mentioned general method, an organic EL devices emitting fluorescence and having the device structure as shown in the FIGURE. From the bottom layer 10 to the top layer 80, the following components were produced: ITO/HAT-CN(20 nm)/NPB (110 nm)/Emitting host material doped with 5% Emitting guest material (30 nm)/HB3(10 nm)/ET2 doped with 50% LiQ(35 nm)/LiQ(1 nm)/A1(160 nm). In the device illustrated in the FIGURE, the hole injection layer 20 (HAT-CN) is deposited onto the transparent electrode 10 (ITO). The hole transport layer 30 (NPB) is deposited onto the hole injection layer 20. The emitting layer 40 is deposited onto the hole transport layer 30. The emitting layer 40 may comprise an emitting host material and an emitting guest (dopant) material, as shown in, for example, Table 1. The emitting host material may be doped with about 5% emitting guest material. The emitting layer 40 may have a thickness of about 30 nm.

The hole blocking layer 50 (HB3) is deposited onto the emitting layer 40. The electron transport layer 60 (ET2 doped with 50% LiQ) is deposited onto the hole blocking layer 50. The electron injection layer 70 (LiQ) is deposited onto the electron transport layer 60. The metal electrode 80 (Al) is deposited onto the electron injection layer 70. The I-V-B (at 1000 nits) test reports of these organic EL devices are summarized in Table 1 below. The half-life time is defined that the initial luminance of 1000 cd/m² has dropped to half.

TABLE 1

| Emitting | Emitting | Driving | Current | | Half-life |
|---|---|---|---|---|---|
| Host | Dopant | Voltage | Efficiency | Device | time |
| Material | Material | (V) | (cd/A) | Color | (hours) |
| H1 | EX7 | 3.4 | 6.5 | blue | 280 |
| H1 | EX16 | 3.5 | 6.3 | blue | 250 |
| H1 | EX42 | 4.4 | 5.0 | blue | 130 |
| H1 | EX114 | 3.6 | 6.1 | blue | 220 |
| H1 | EX130 | 3.6 | 5.9 | blue | 200 |
| H1 | EX138 | 4.1 | 5.3 | blue | 160 |
| H1 | EX164 | 3.8 | 5.7 | blue | 180 |
| H1 | EX173 | 5.2 | 52.4 | green | 500 |
| H1 | EX184 | 5.4 | 51.3 | green | 480 |
| H1 | D1 | 4.0 | 5.3 | blue | 180 |
| H2 | D1 | 3.8 | 5.5 | blue | 210 |
| H1 | D2 | 5.8 | 45.2 | green | 430 |
| H2 | D2 | 5.5 | 46.8 | green | 450 |
| H2 | EX7 | 3.3 | 6.9 | blue | 340 |
| H2 | EX16 | 3.4 | 6.7 | blue | 310 |
| H2 | EX42 | 4.2 | 5.1 | blue | 190 |
| H2 | EX114 | 3.5 | 6.4 | blue | 290 |
| H2 | EX130 | 3.6 | 6.2 | blue | 270 |
| H2 | EX138 | 3.8 | 5.9 | blue | 240 |
| H2 | EX164 | 4.0 | 5.7 | blue | 230 |
| H2 | EX173 | 5.1 | 53.9 | green | 520 |
| H2 | EX184 | 5.3 | 52.1 | green | 500 |
| EX1 | D1 | 3.2 | 7.1 | blue | 300 |
| EX2 | D1 | 3.2 | 7.2 | blue | 320 |
| EX5 | D1 | 3.4 | 6.6 | blue | 280 |
| EX6 | D1 | 3.3 | 6.7 | blue | 290 |
| EX18 | D1 | 3.6 | 5.8 | blue | 240 |
| EX23 | D1 | 3.4 | 6.5 | blue | 270 |
| EX37 | D1 | 3.4 | 6.5 | blue | 270 |
| EX39 | D1 | 4.0 | 5.0 | blue | 180 |
| EX53 | D1 | 3.8 | 5.5 | blue | 220 |
| EX58 | D1 | 3.7 | 5.7 | blue | 230 |
| EX77 | D1 | 4.1 | 4.9 | blue | 170 |
| EX88 | D1 | 3.8 | 5.6 | blue | 220 |
| EX92 | D1 | 4.2 | 4.7 | blue | 160 |
| EX110 | D1 | 3.1 | 7.3 | blue | 340 |
| EX115 | D1 | 3.3 | 6.8 | blue | 290 |
| EX131 | D1 | 3.5 | 6.2 | blue | 250 |
| EX132 | D1 | 3.5 | 6.2 | blue | 260 |
| EX140 | D1 | 4.1 | 4.8 | blue | 160 |
| EX146 | D1 | 3.1 | 7.2 | blue | 320 |
| EX157 | D1 | 3.3 | 6.9 | blue | 300 |
| EX171 | D1 | 3.5 | 6.3 | blue | 260 |
| EX182 | D1 | 3.6 | 6.0 | blue | 250 |
| EX195 | D1 | 3.6 | 5.9 | blue | 240 |

In Table 1, the organic compound of formula (1) used as a fluorescent blue host or dopant material may exhibit better performance than the prior art materials. In particular, an organic EL device of the present invention comprises an organic compound of formula (1) as a dopant material or a host material to collocate with a host material H1 or H2 or a dopant material D1, thereby lowering a driving voltage, improving luminance, or increasing a current efficiency or a half-life time under the same voltage of the organic EL device.

The emitting layer 40 may comprise an emitting host material and an emitting guest (dopant) material, as shown in, for example, Table 2. The I-V-B (at 1000 nits) test reports of these organic EL devices are summarized in Table 2 below. The half-life time is defined that the initial luminance of 1000 cd/m² has dropped to half.

TABLE 2

| Emitting | Emitting | Driving | Current | | |
|---|---|---|---|---|---|
| Host | Dopant | Voltage | Efficiency | Device | Half-life time |
| Material | Material | (V) | (cd/A) | Color | (hours) |
| H1 | D1 | 4.0 | 5.3 | blue | 180 |
| H2 | D1 | 3.8 | 5.5 | blue | 210 |
| EX1 | D1 | 3.2 | 7.1 | blue | 300 |
| EX2 | D1 | 3.2 | 7.2 | blue | 320 |
| EX110 | D1 | 3.1 | 7.3 | blue | 340 |
| EX146 | D1 | 3.1 | 7.2 | blue | 320 |

Obviously, many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

The invention claimed is:

1. An organic compound represented by the following formula (1): 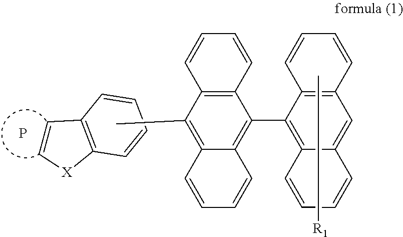
wherein X is a divalent bridge selected from the group consisting of O, S, and SiR₂R₃; P represents a substituted or unsubstituted fused ring hydrocarbons unit having two, three or four rings; and R₁ to R₃ independently represent a hydrogen atom, a halide, a substituted or unsubstituted alkyl group having 1 to 30 carbon atoms, a substituted or unsubstituted alkoxy group having 1 to 30 carbon atoms, a substituted or unsubstituted aryl group having 6 to 30 carbon atoms, a substituted or unsubstituted aralkyl group having 7 to 30 carbon atoms, or a substituted or unsubstituted heteroaryl group having 3 to 30 carbon atoms.

2. The organic compound according to claim 1, wherein the substituted alkyl group, the substituted alkoxy group, the substituted aryl group, the substituted aralkyl group, or the substituted heteroaryl group is substituted by a halide, an alkyl group, an alkoxy group, or an aryl group.

3. The organic compound according to claim 1, wherein the organic compound is selected from the group consist of:

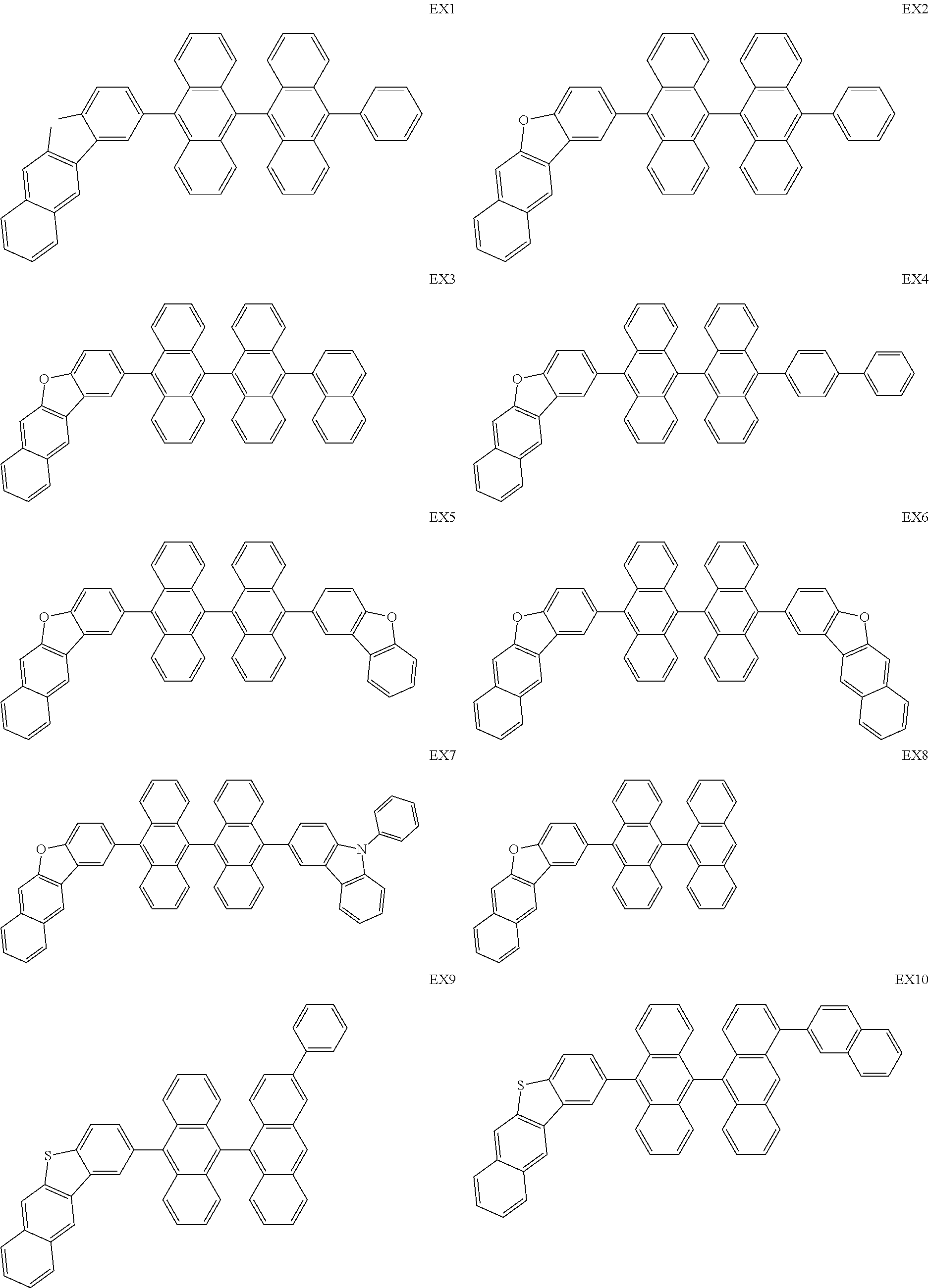

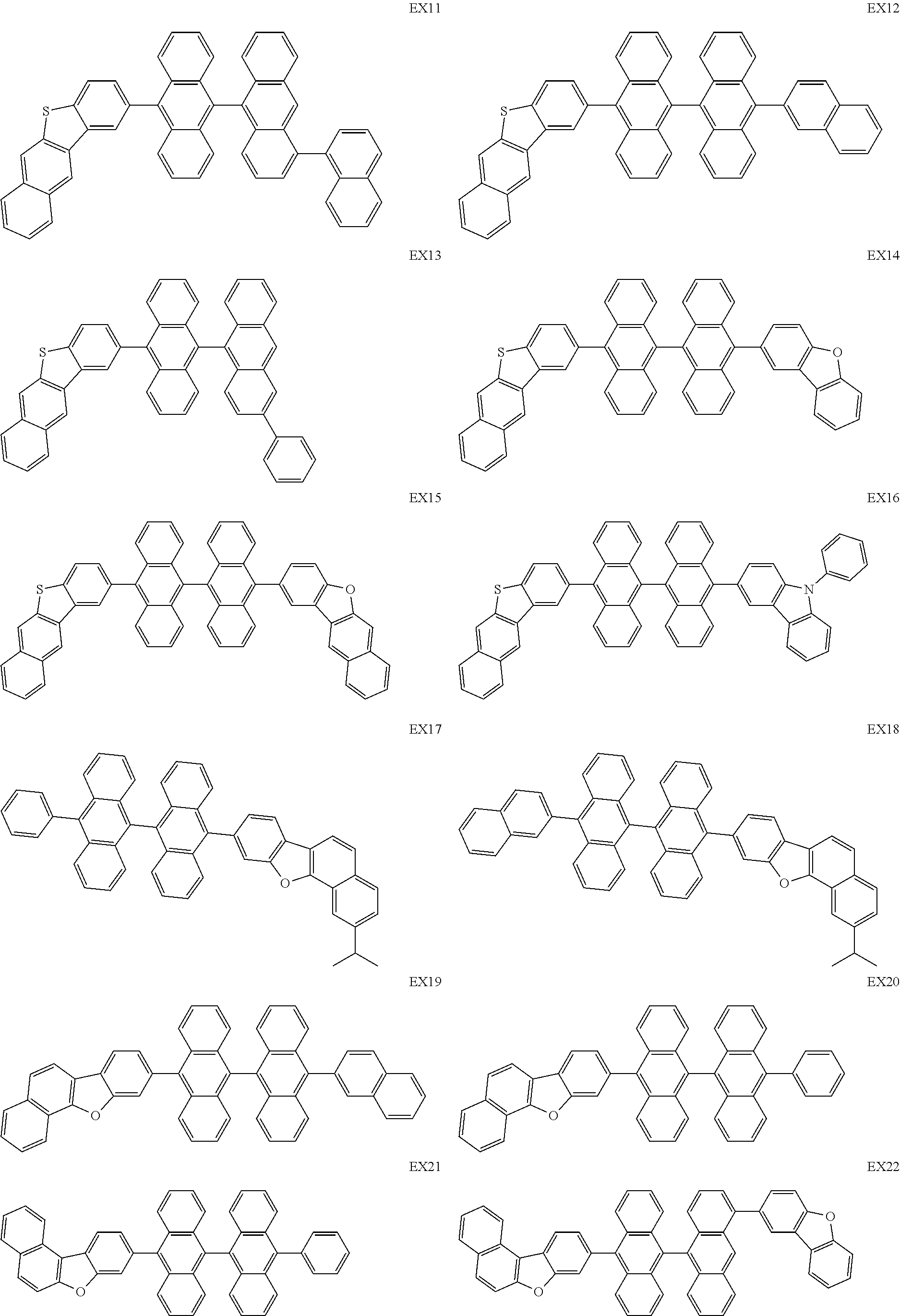

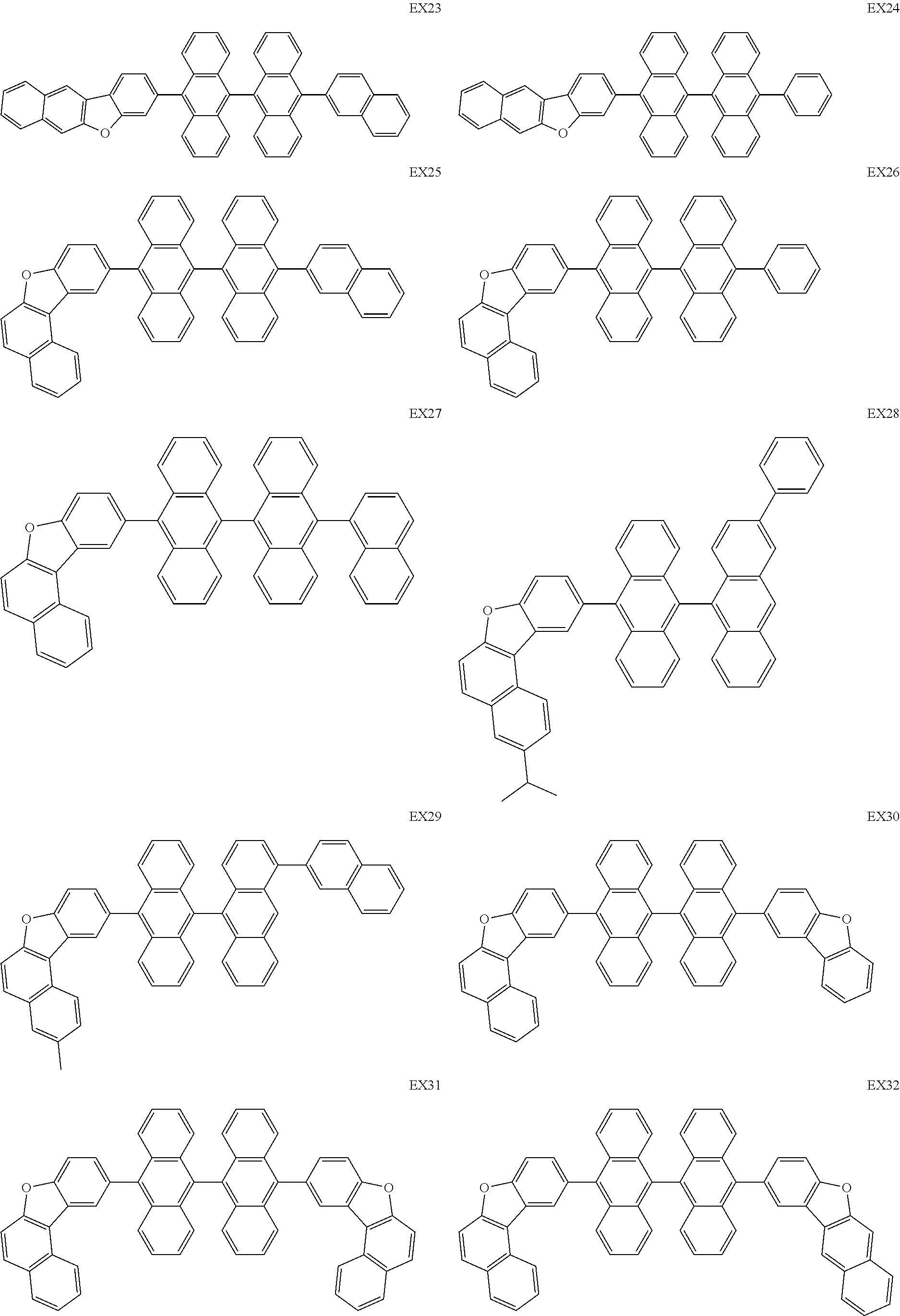

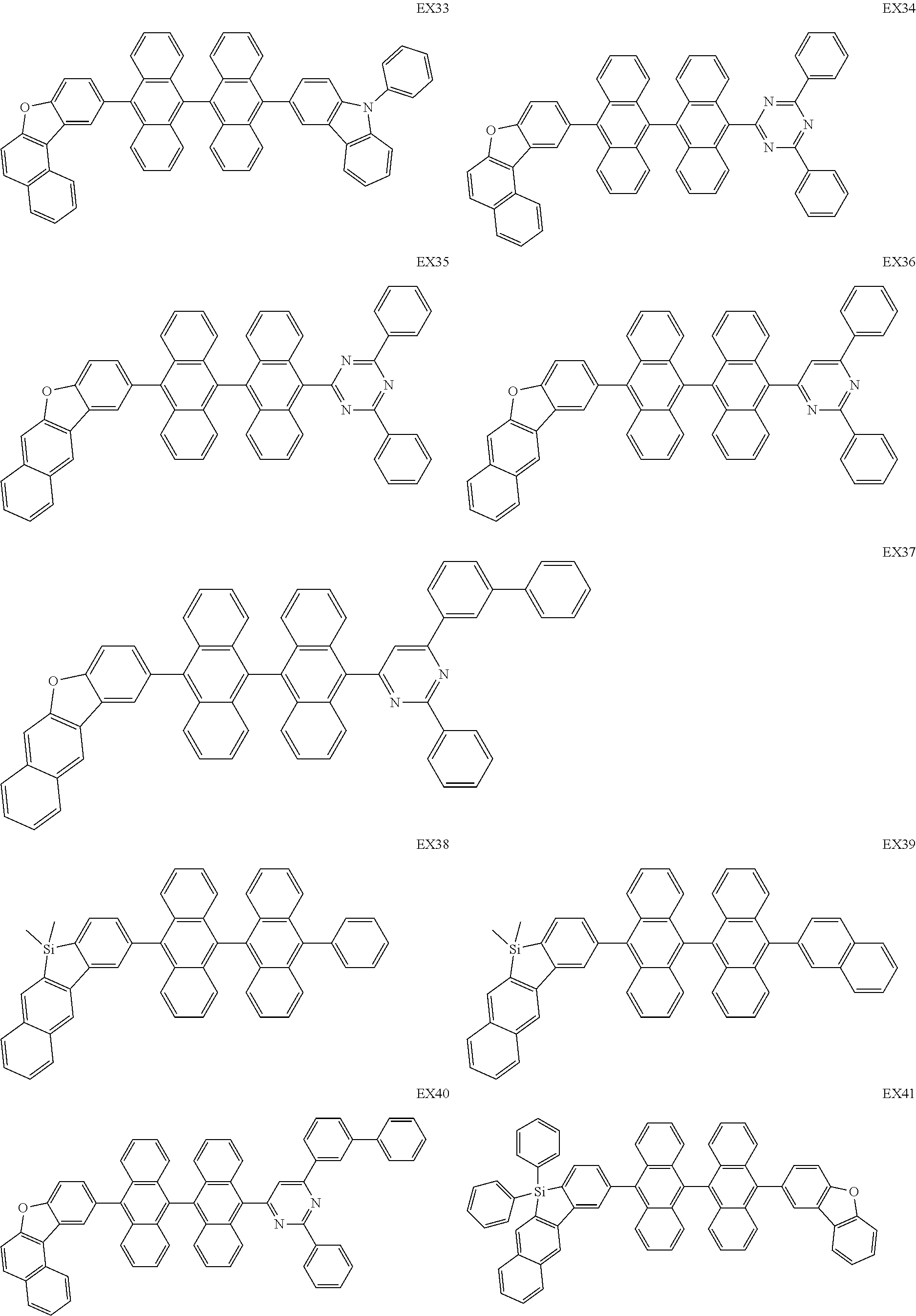

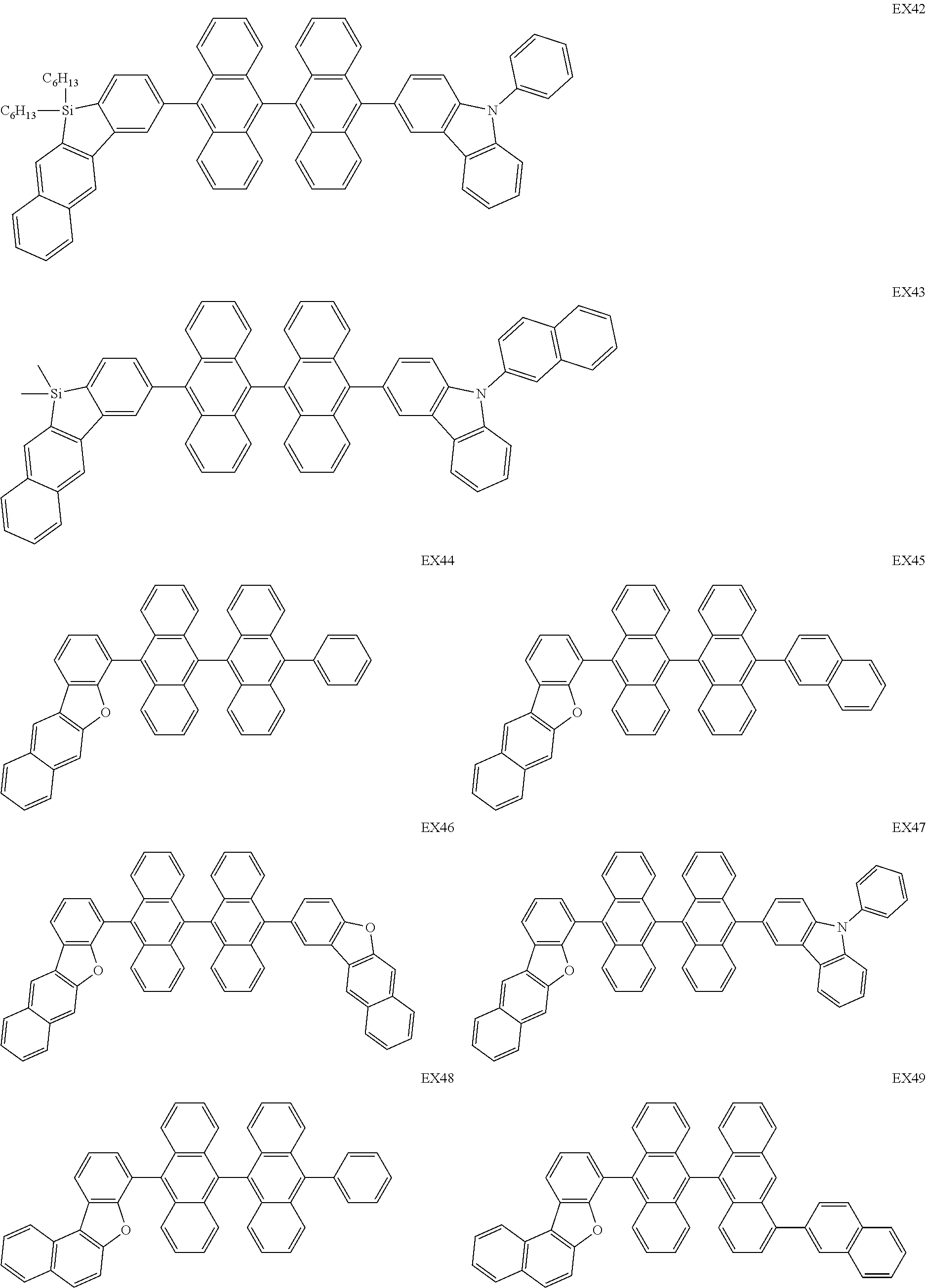

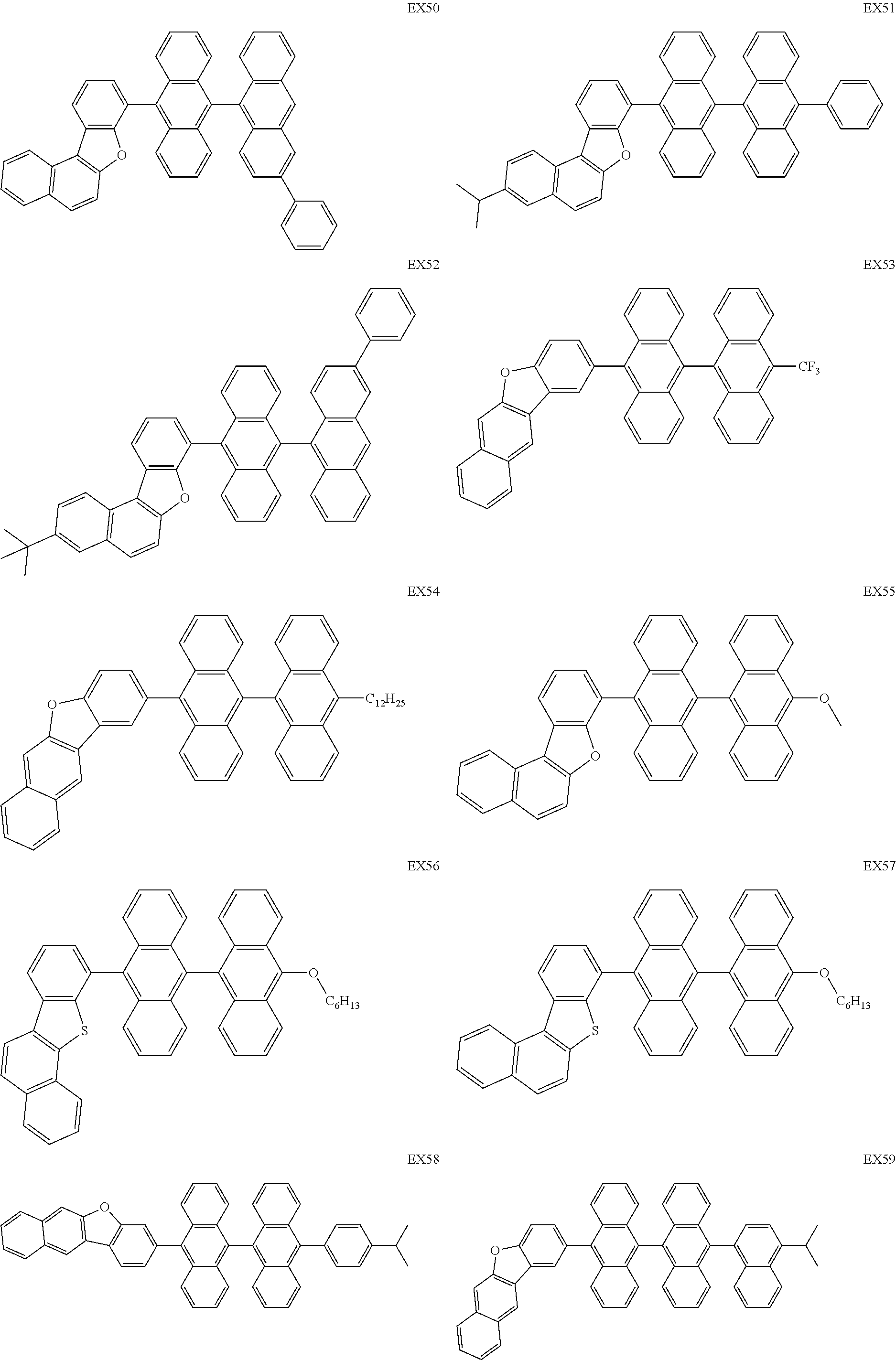

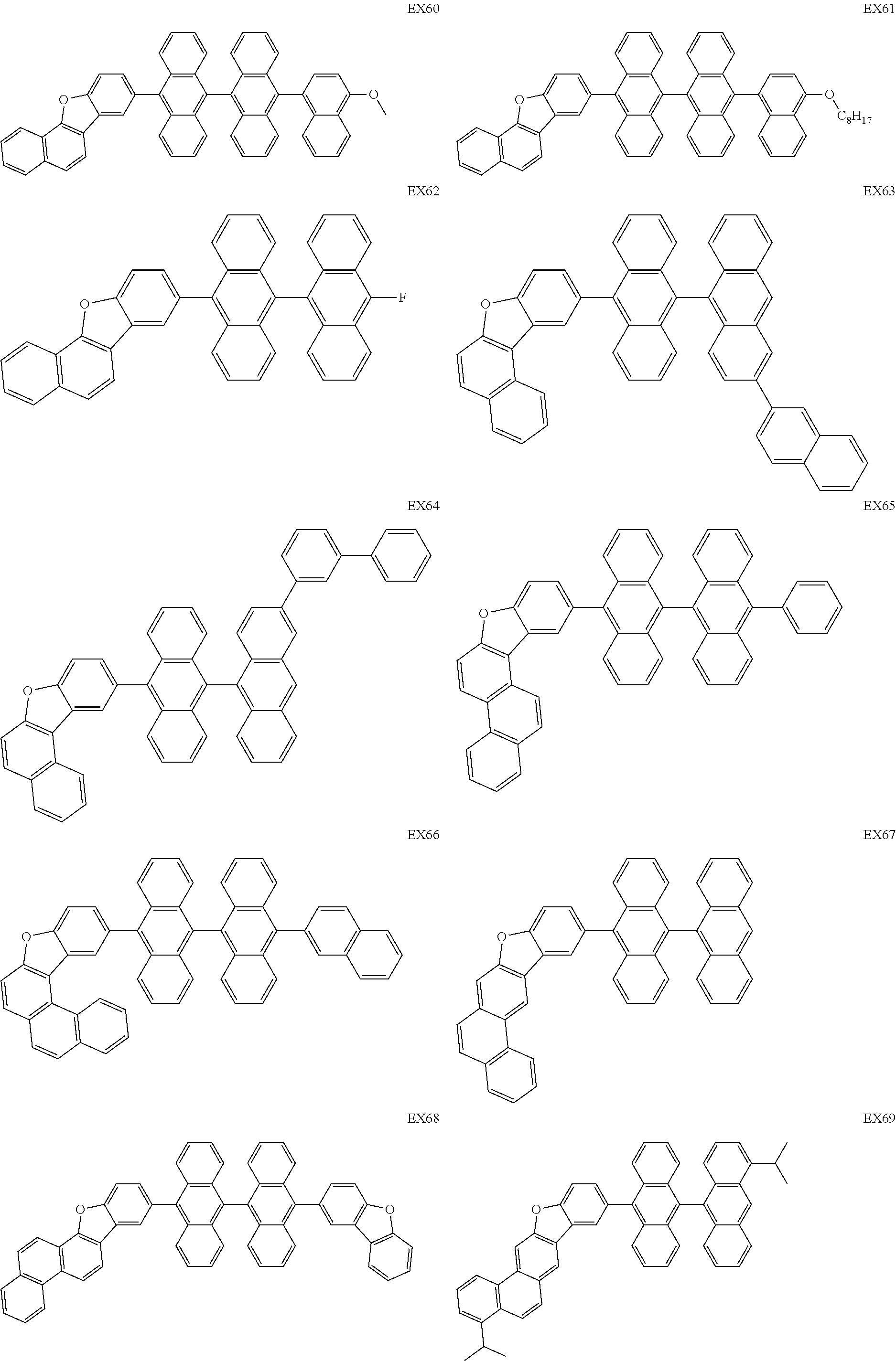

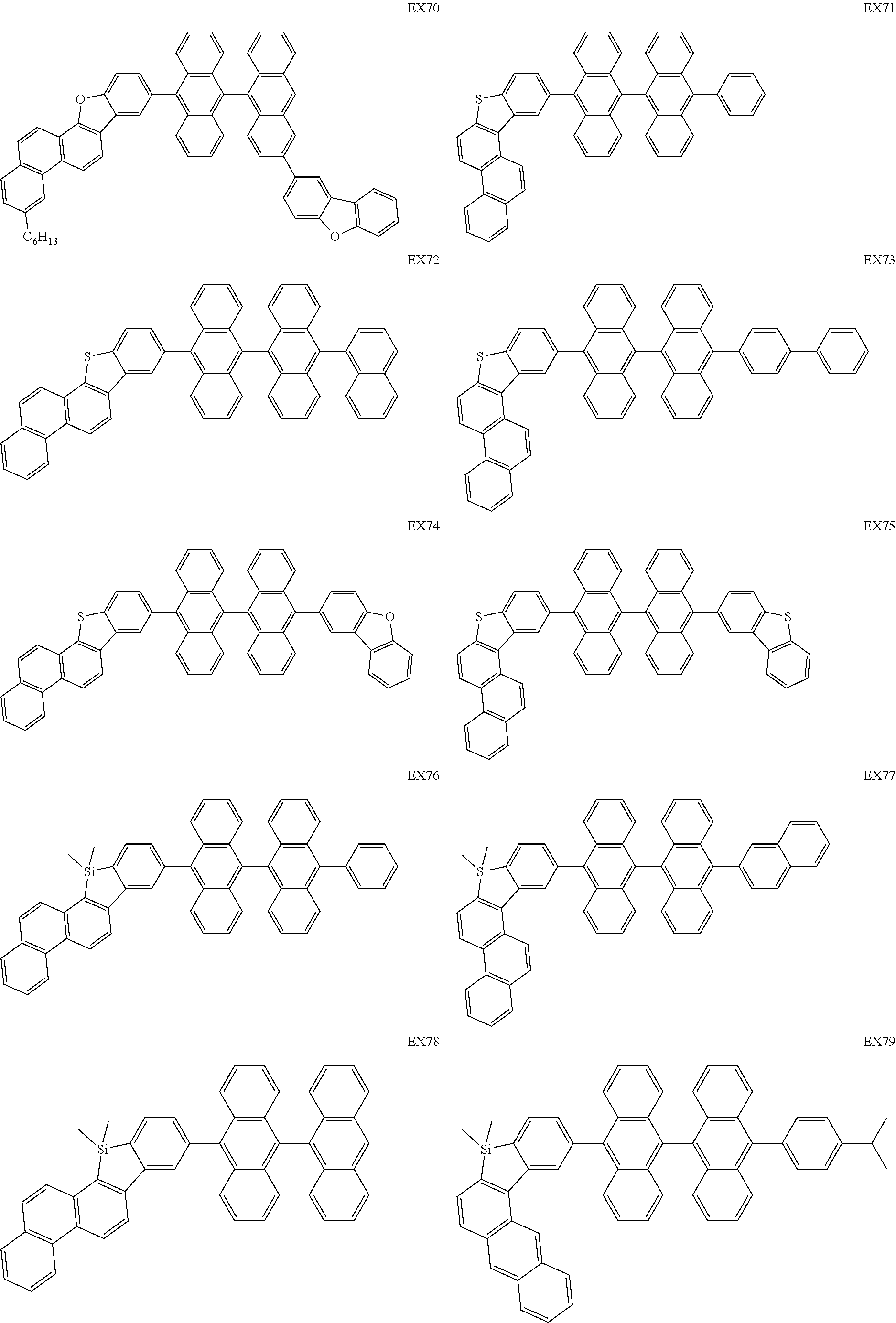

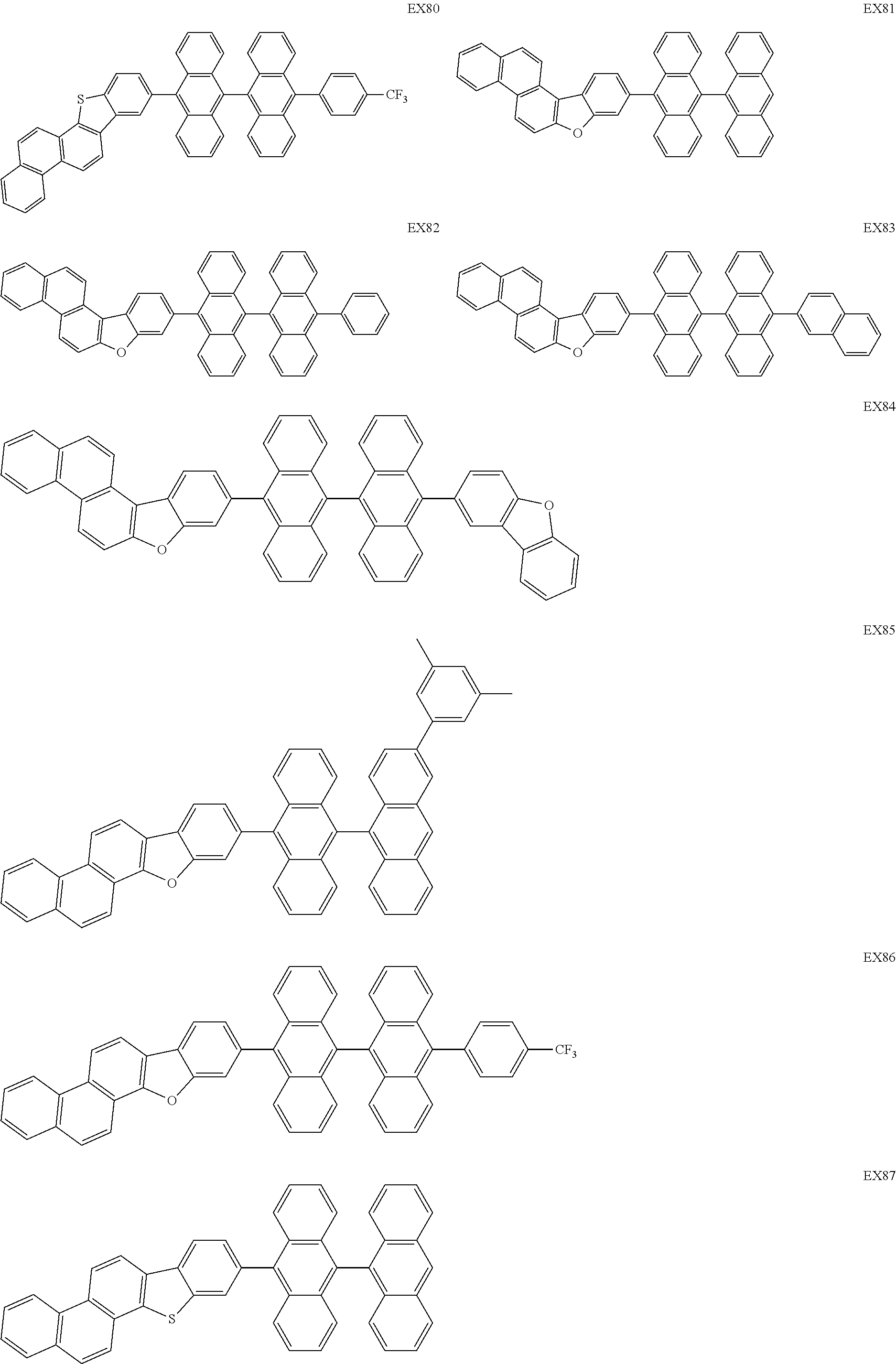

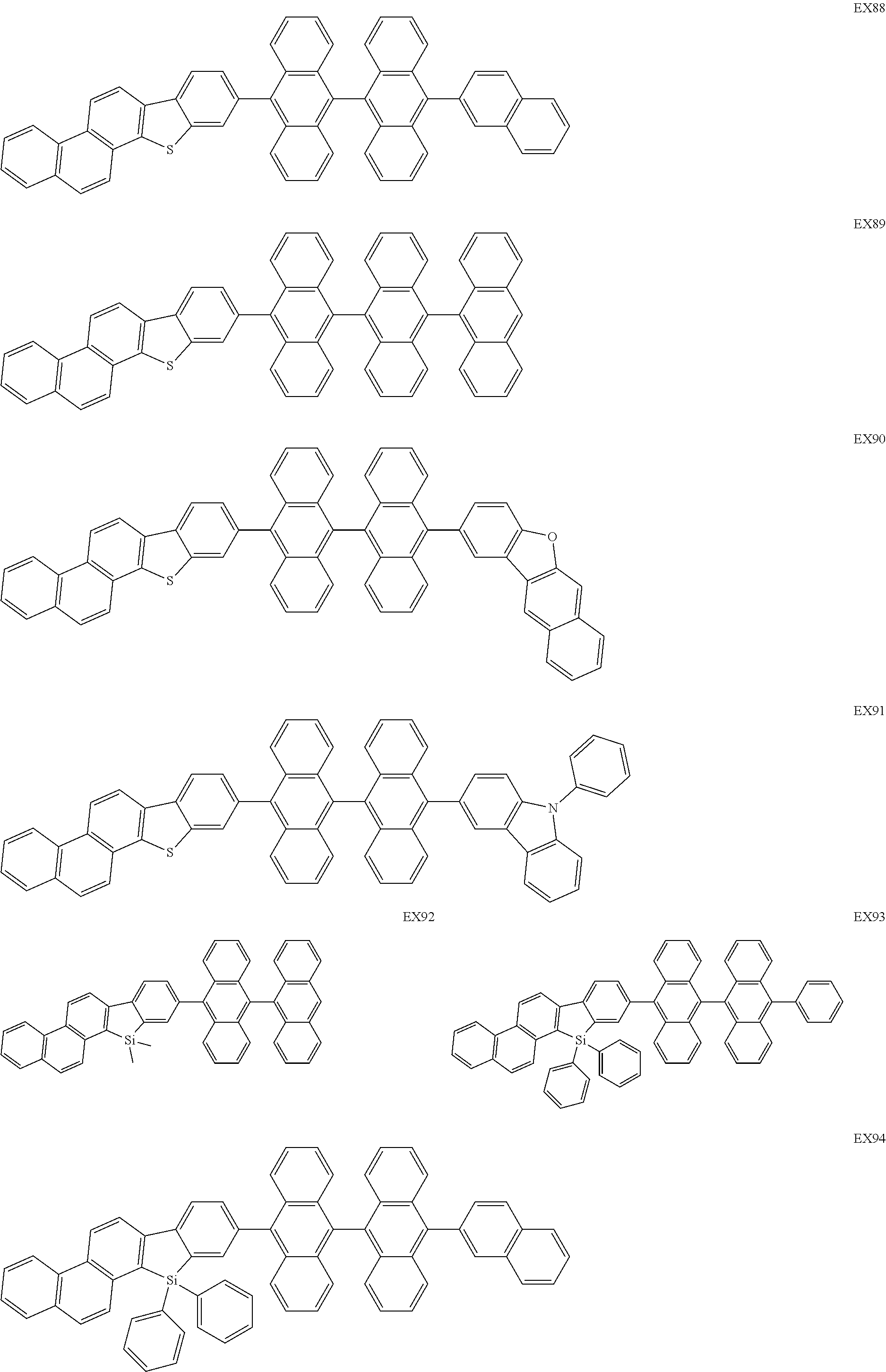

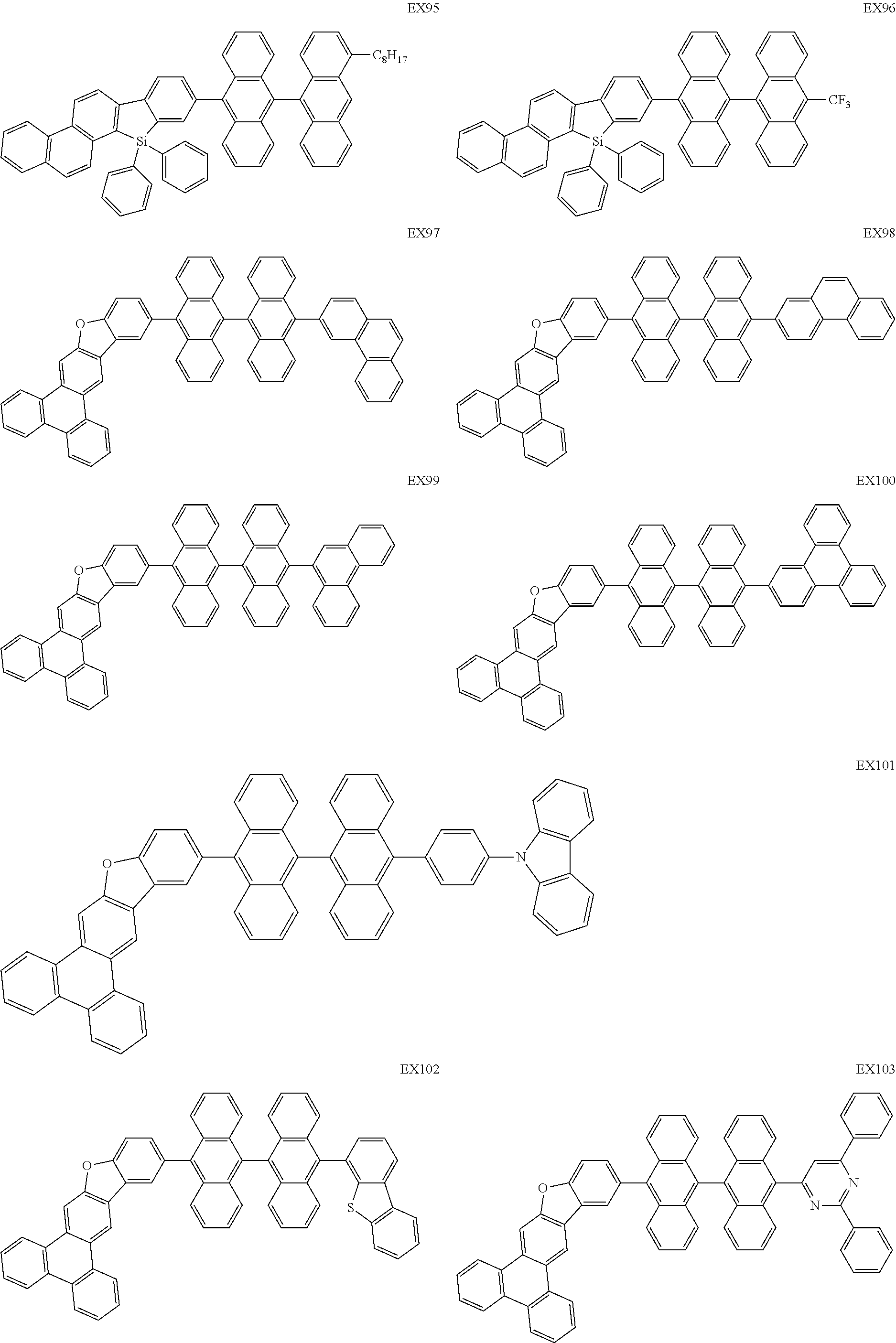

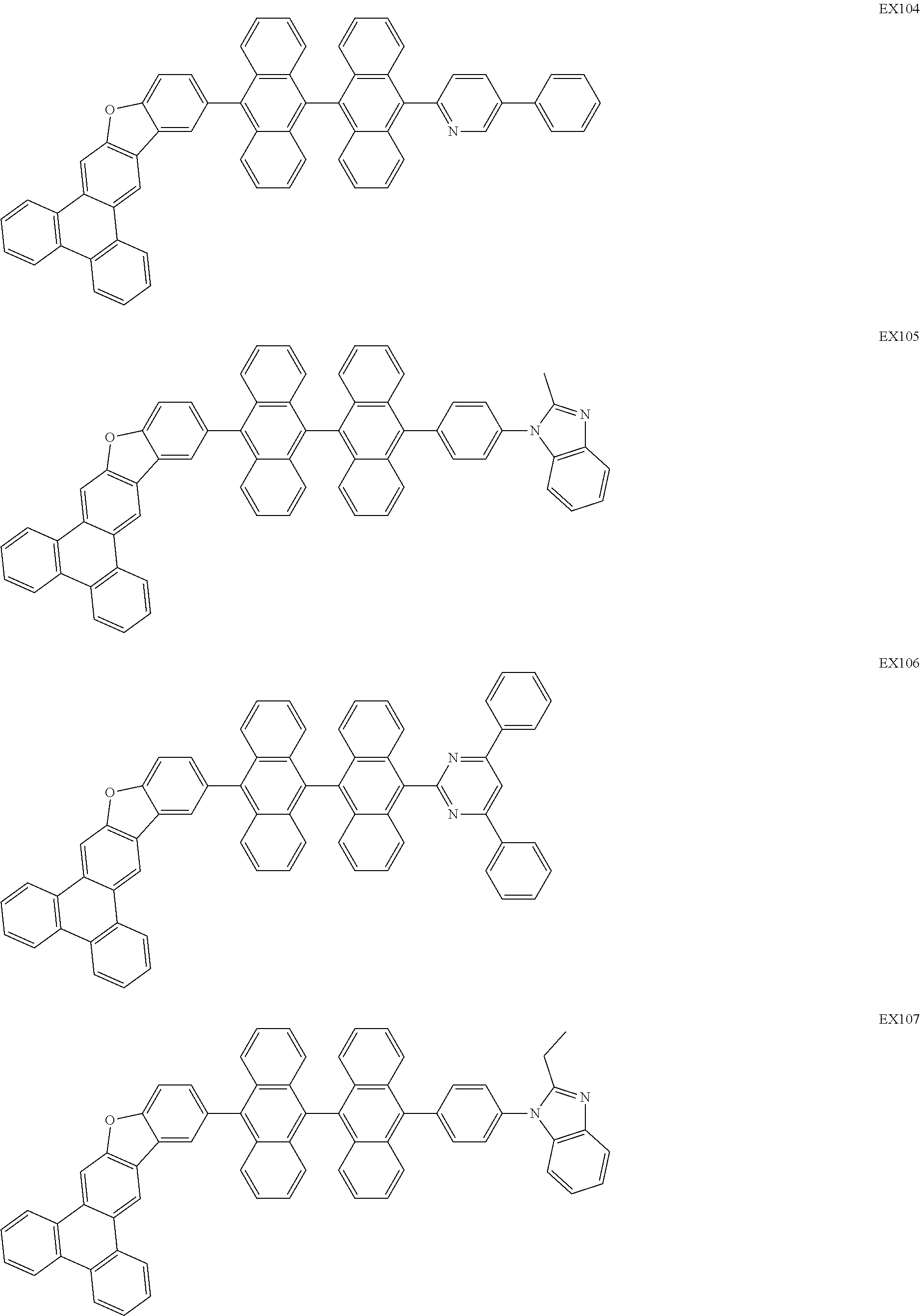 [SMILES: c1ccc(-c2ccc(-c3c4ccccc4c(-c4c5ccccc5c(-c5ccc6oc7cc8c9ccccc9c9ccccc9c8cc7c6c5)c5ccccc45)c4ccccc34)nc2)cc1 ; Cc1nc2ccccc2n1-c1ccc(-c2c3ccccc3c(-c3c4ccccc4c(-c4ccc5oc6cc7c8ccccc8c8ccccc8c7cc6c5c4)c4ccccc34)c3ccccc23)cc1 ; c1ccc(-c2cc(-c3ccccc3)nc(-c3c4ccccc4c(-c4c5ccccc5c(-c5ccc6oc7cc8c9ccccc9c9ccccc9c8cc7c6c5)c5ccccc45)c4ccccc34)n2)cc1 ; CCc1nc2ccccc2n1-c1ccc(-c2c3ccccc3c(-c3c4ccccc4c(-c4ccc5oc6cc7c8ccccc8c8ccccc8c7cc6c5c4)c4ccccc34)c3ccccc23)cc1]

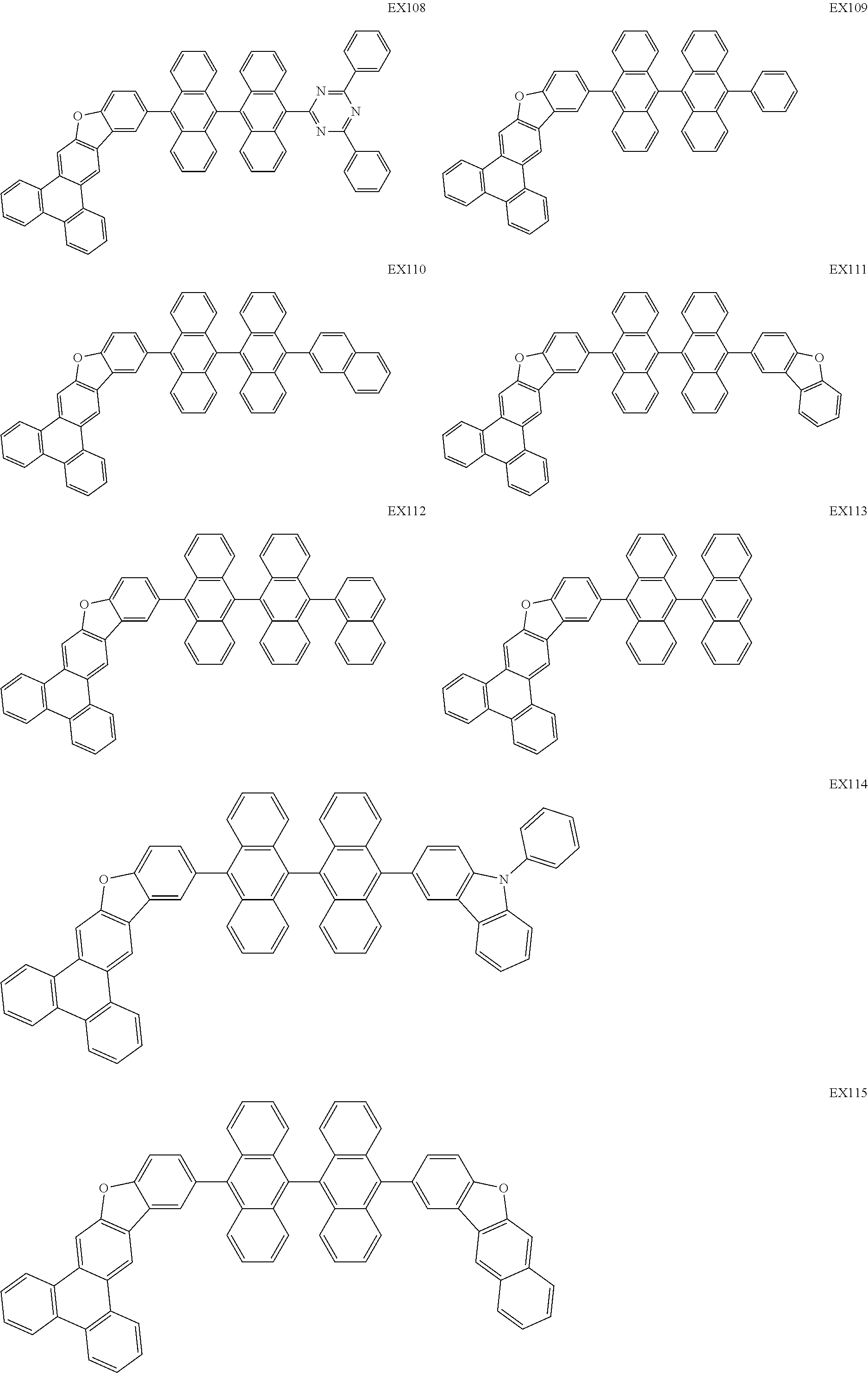

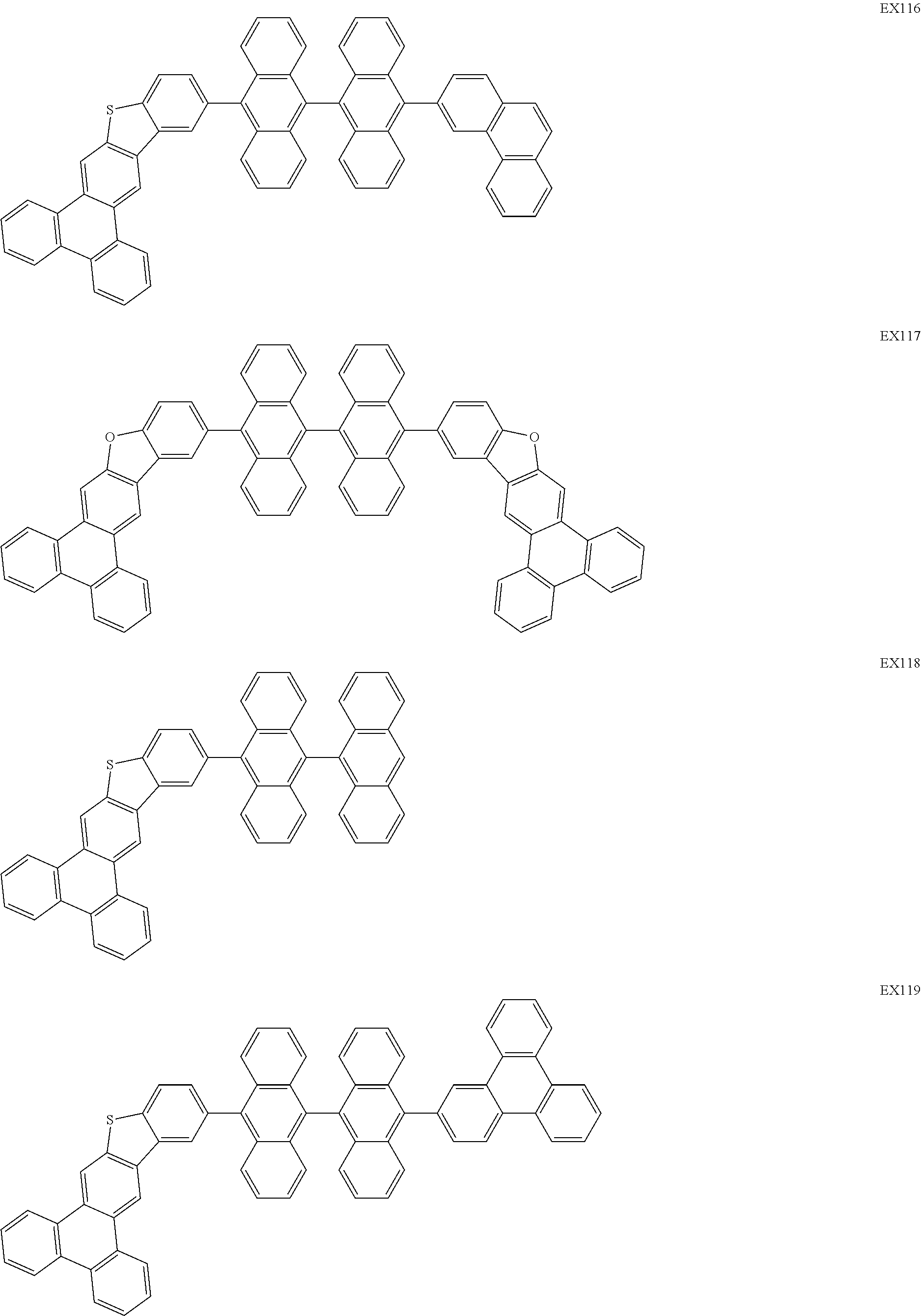 [SMILES: c1ccc2c(c1)ccc1ccc(-c3c4ccccc4c(-c4c5ccccc5c(-c5ccc6sc7cc8c9ccccc9c9ccccc9c8cc7c6c5)c5ccccc45)c4ccccc34)cc12 ; c1ccc2c(-c3c4ccccc4c(-c4ccc5oc6cc7c8ccccc8c8ccccc8c7cc6c5c4)c4ccccc34)c3ccccc3c(-c3ccc4oc5cc6c7ccccc7c7ccccc7c6cc5c4c3)c2c1 ; c1ccc2c(-c3c4ccccc4c(-c4ccc5sc6cc7c8ccccc8c8ccccc8c7cc6c5c4)c4ccccc34)c3ccccc3cc2c1 ; c1ccc2c(-c3c4ccccc4c(-c4ccc5c6ccccc6c6ccccc6c5c4)c4ccccc34)c3ccccc3c(-c3ccc4sc5cc6c7ccccc7c7ccccc7c6cc5c4c3)c2c1]

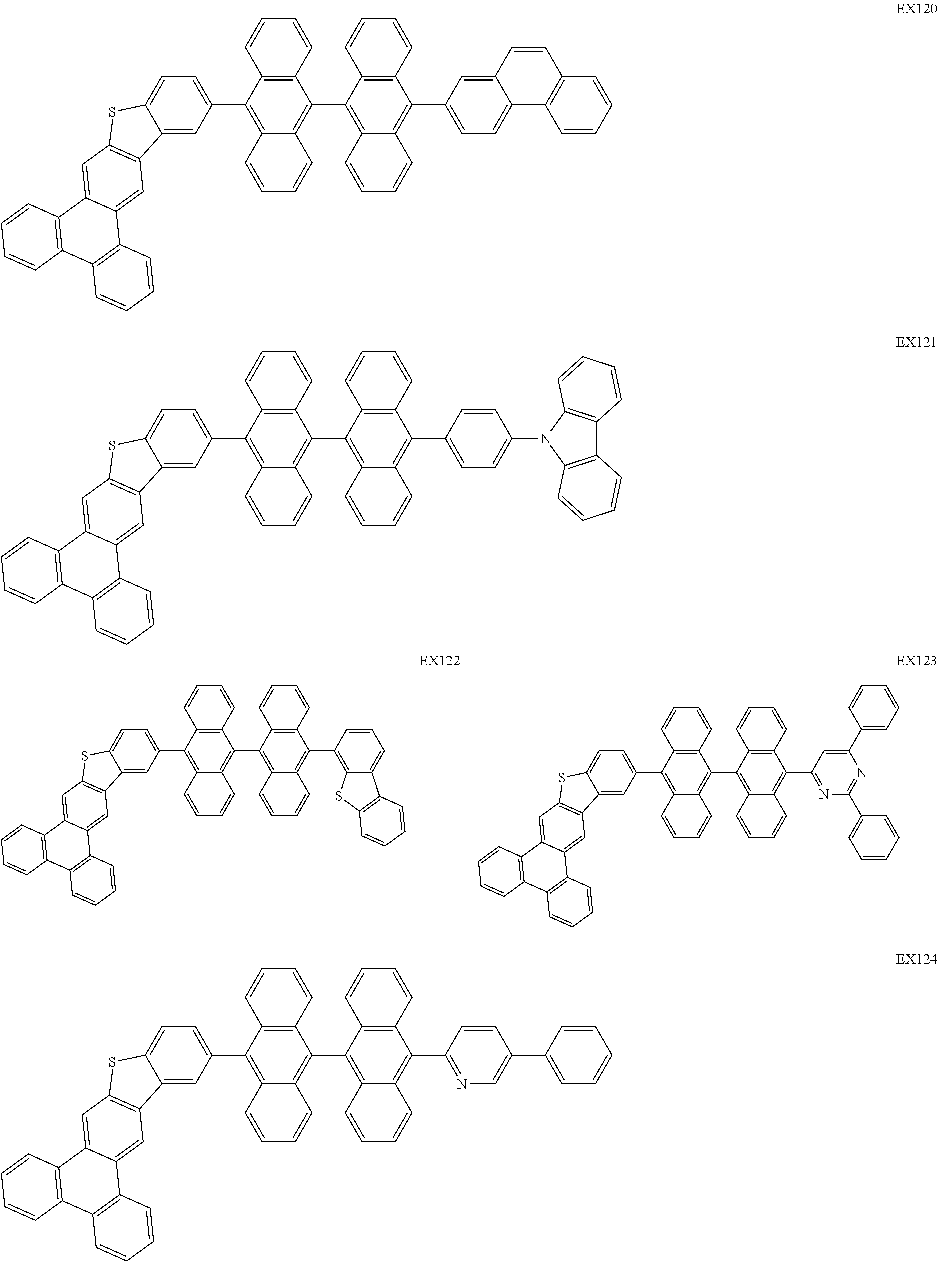

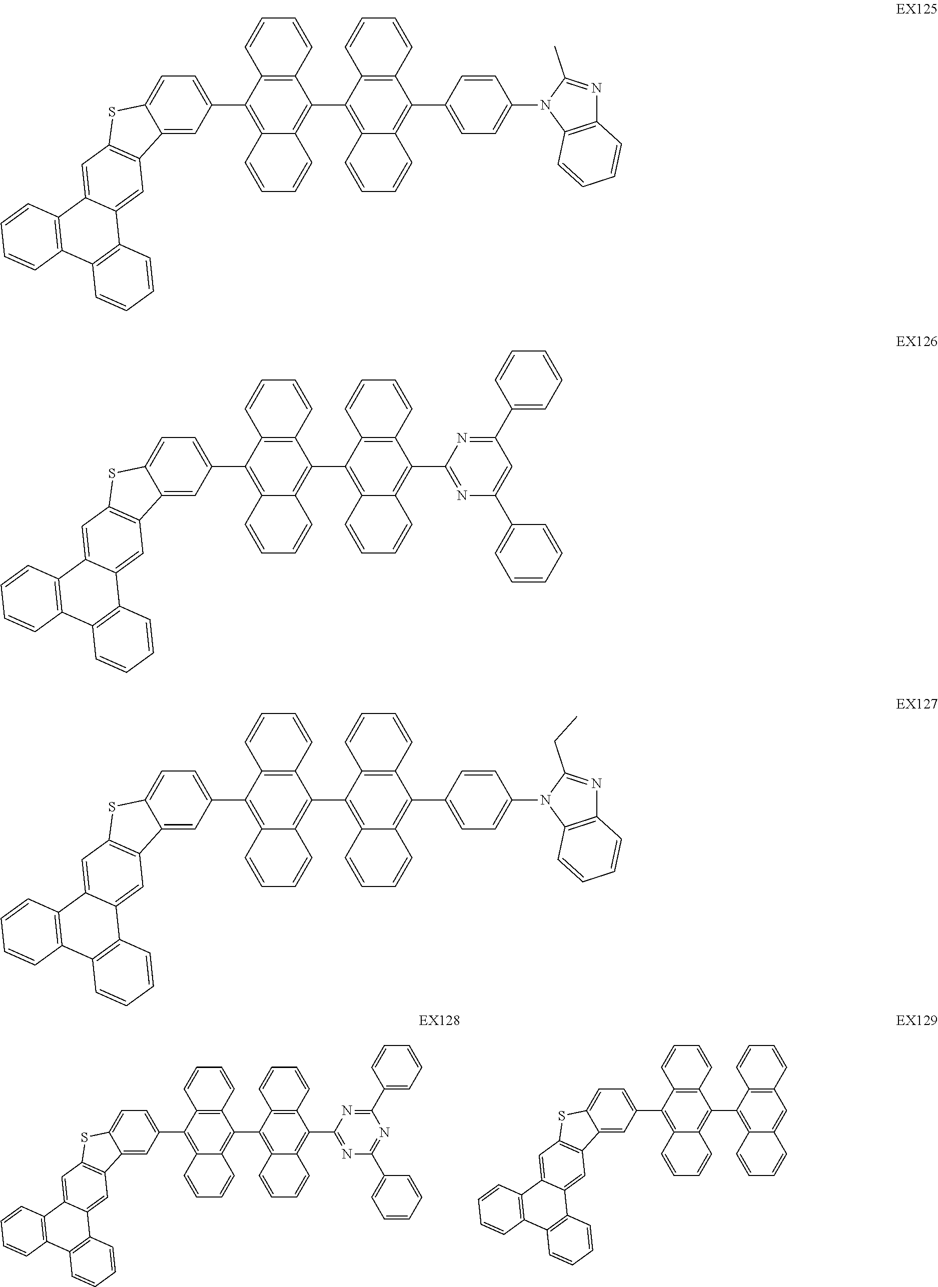

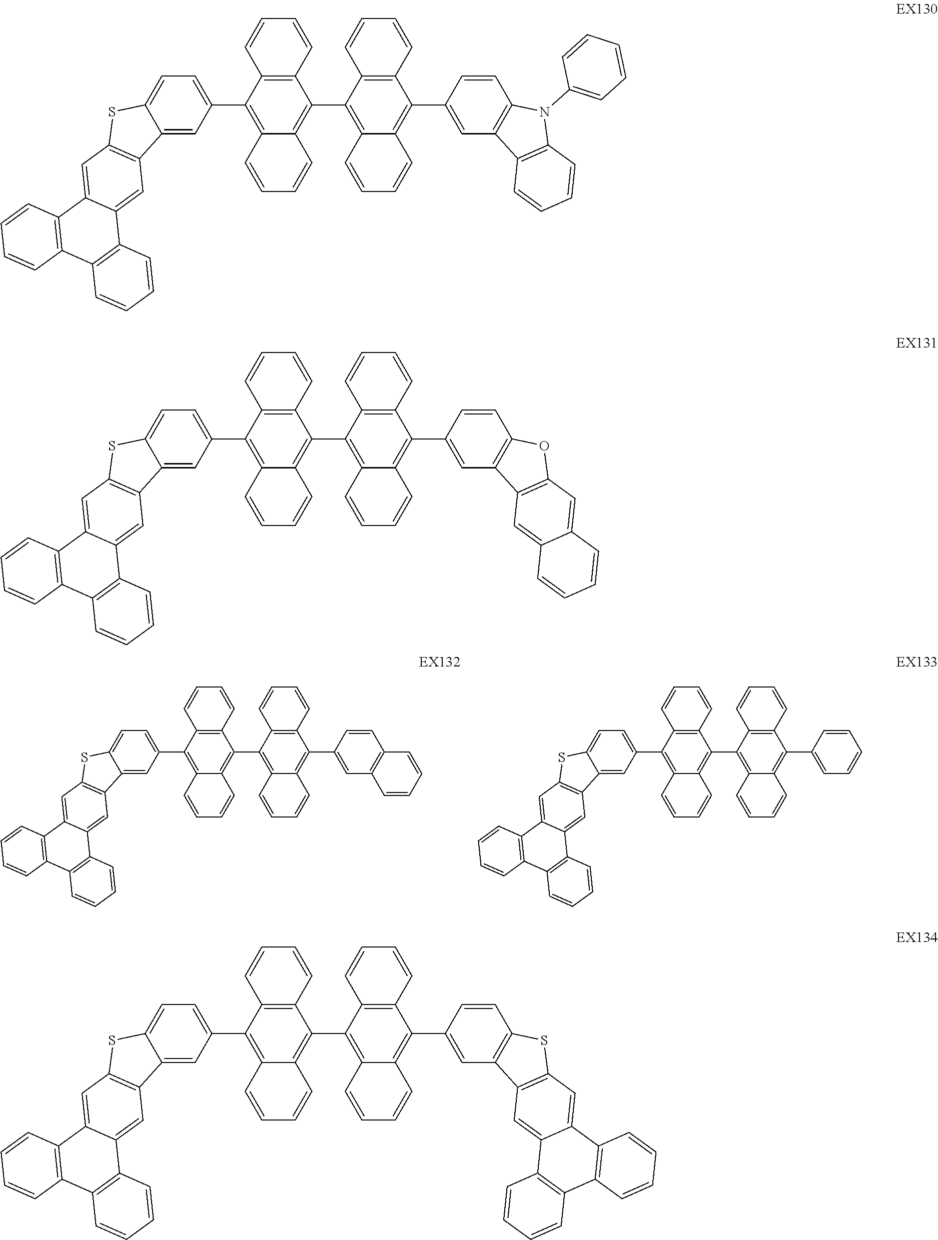

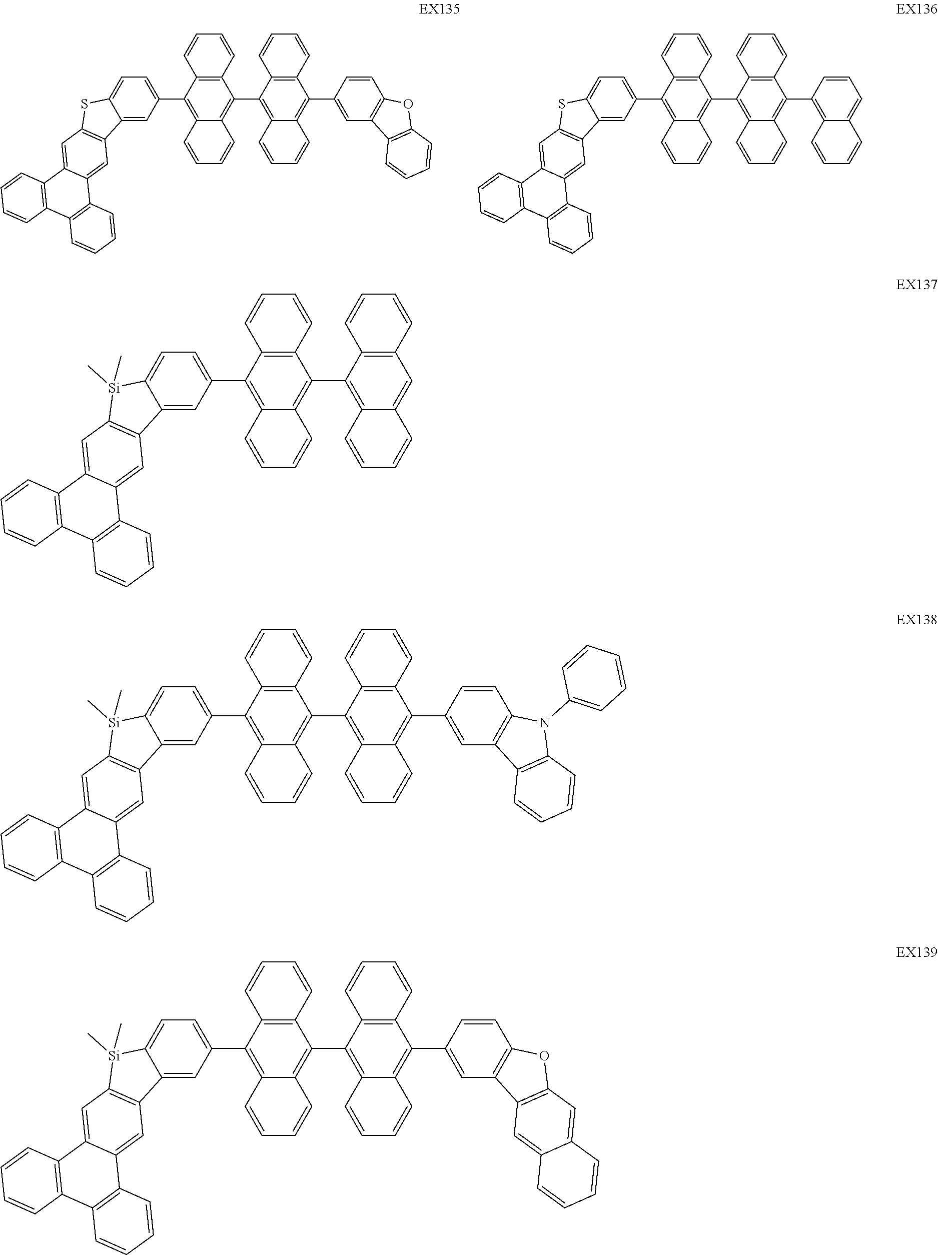

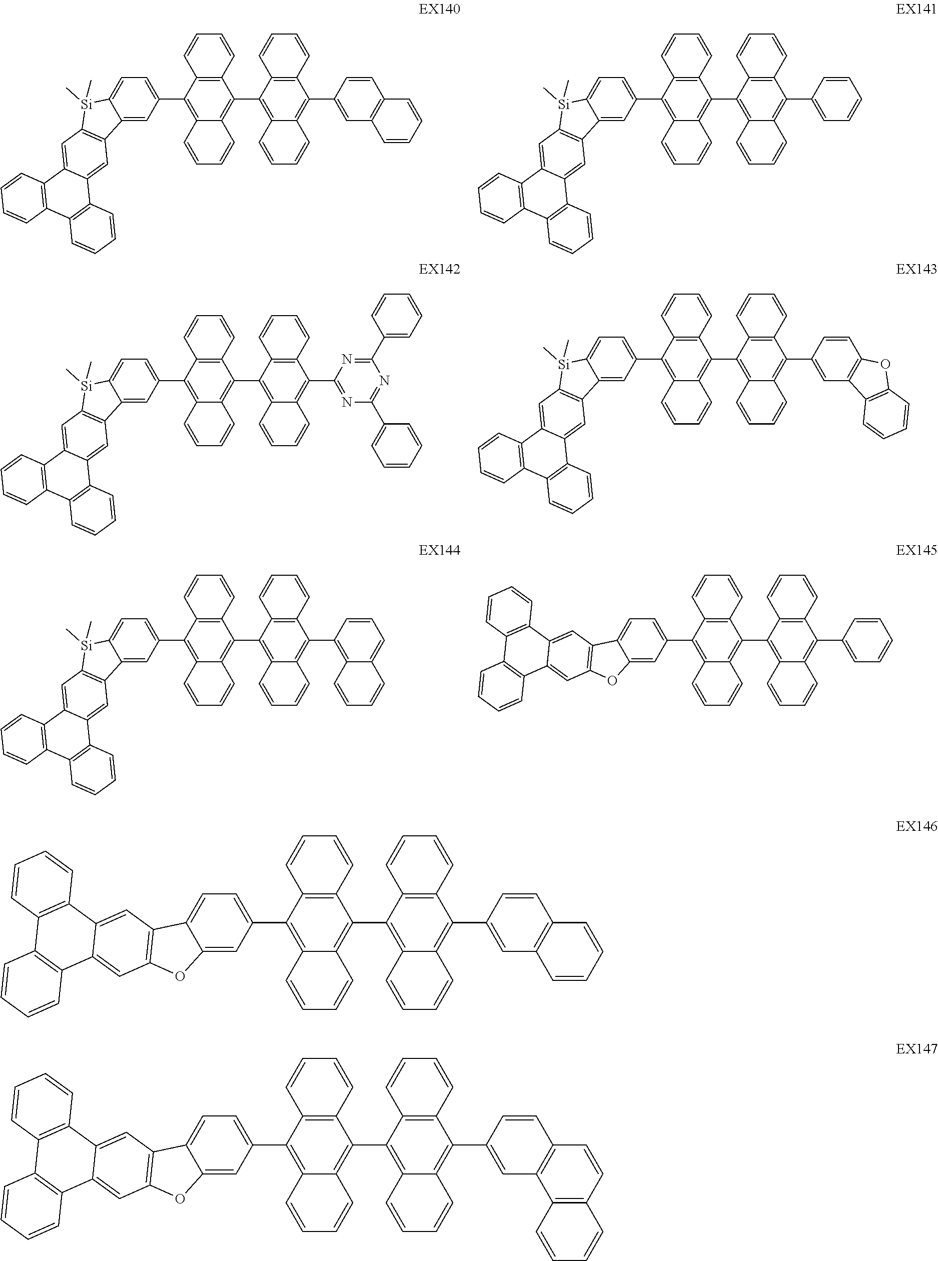

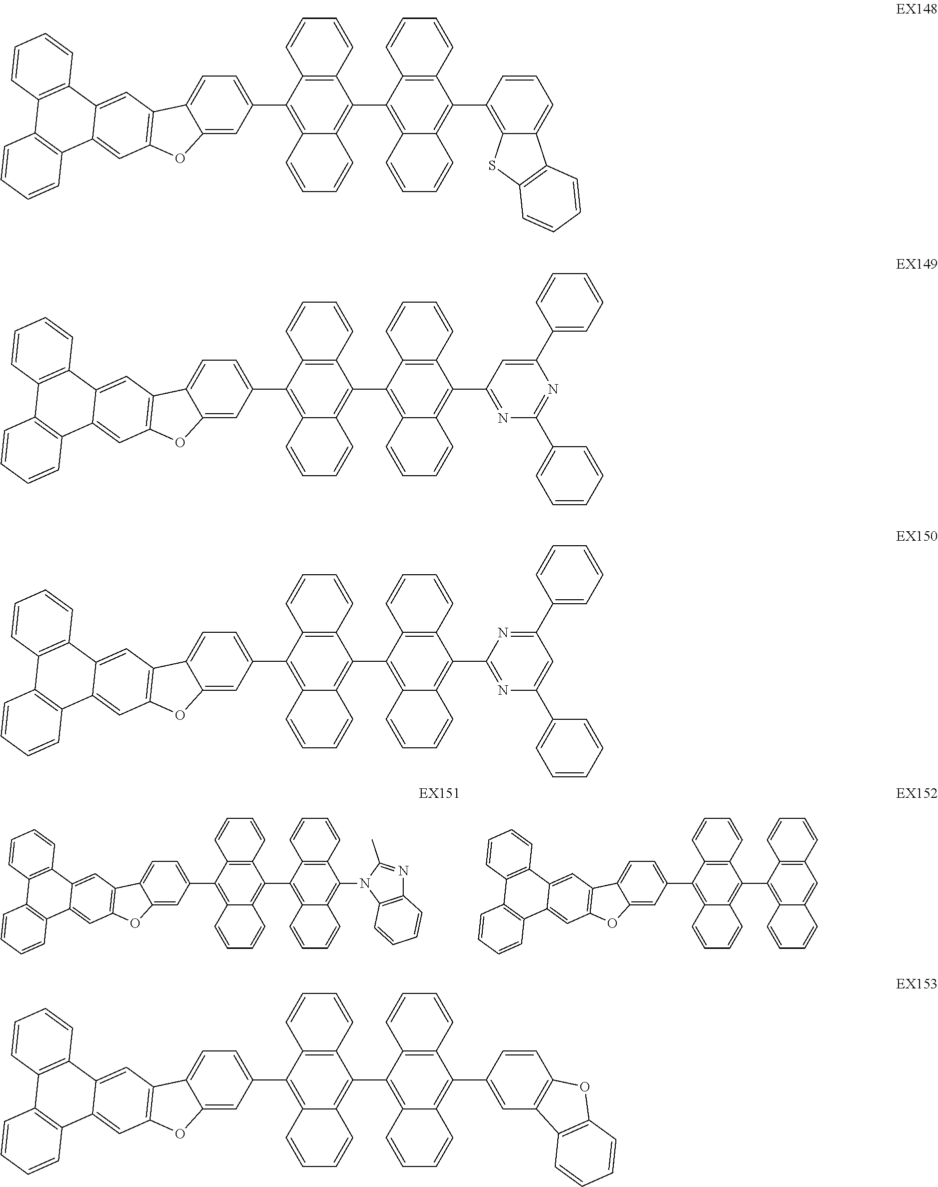

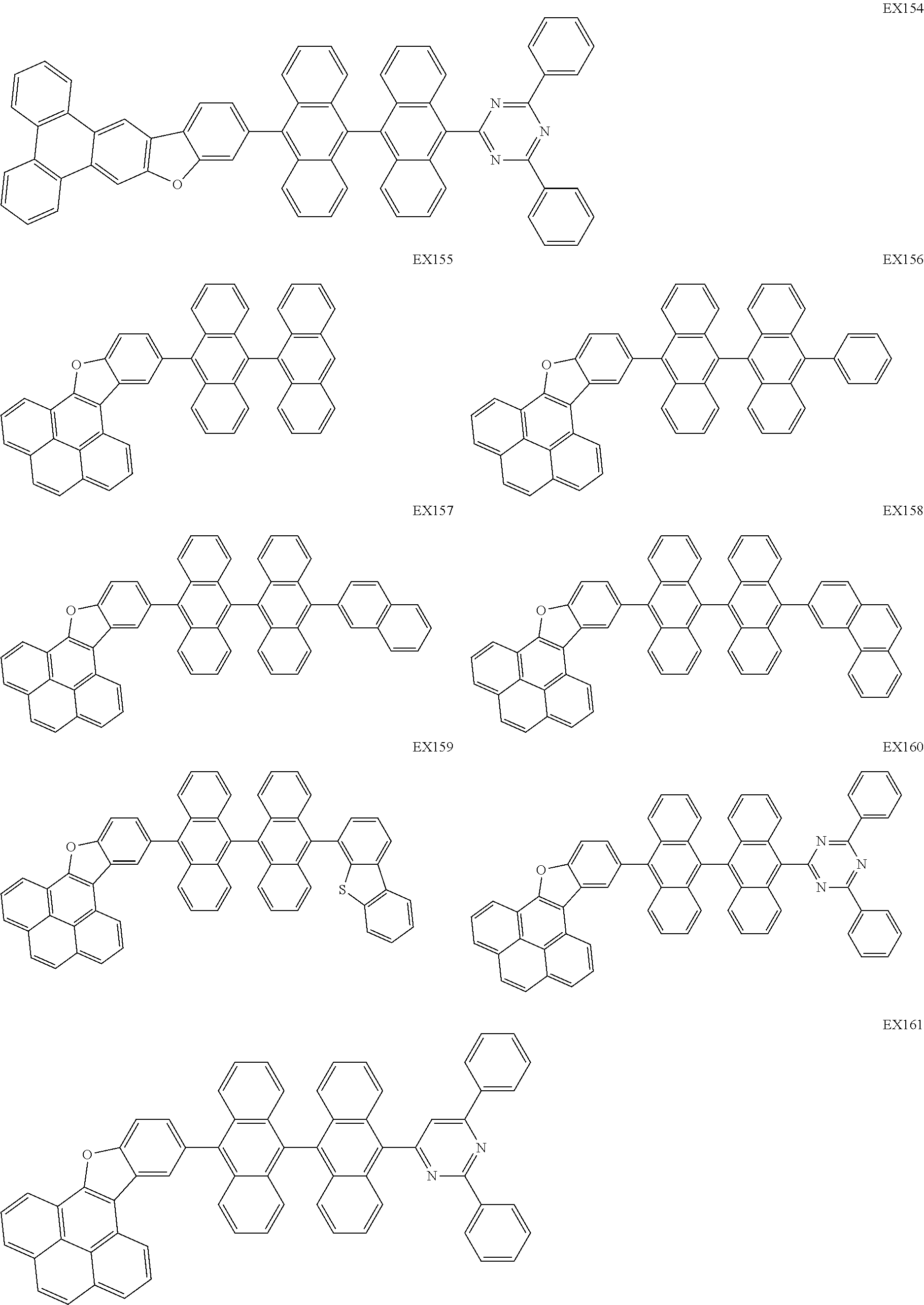

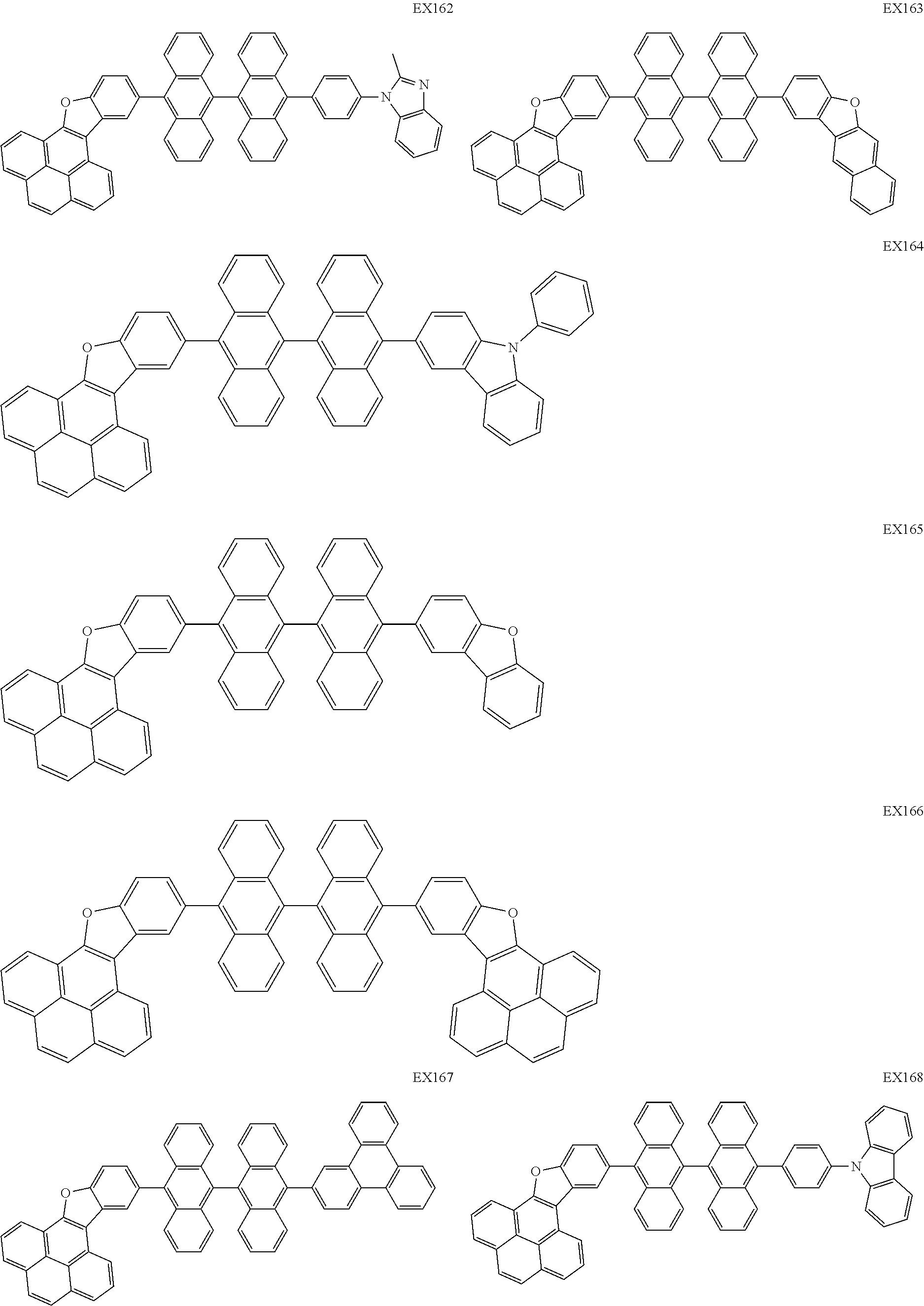

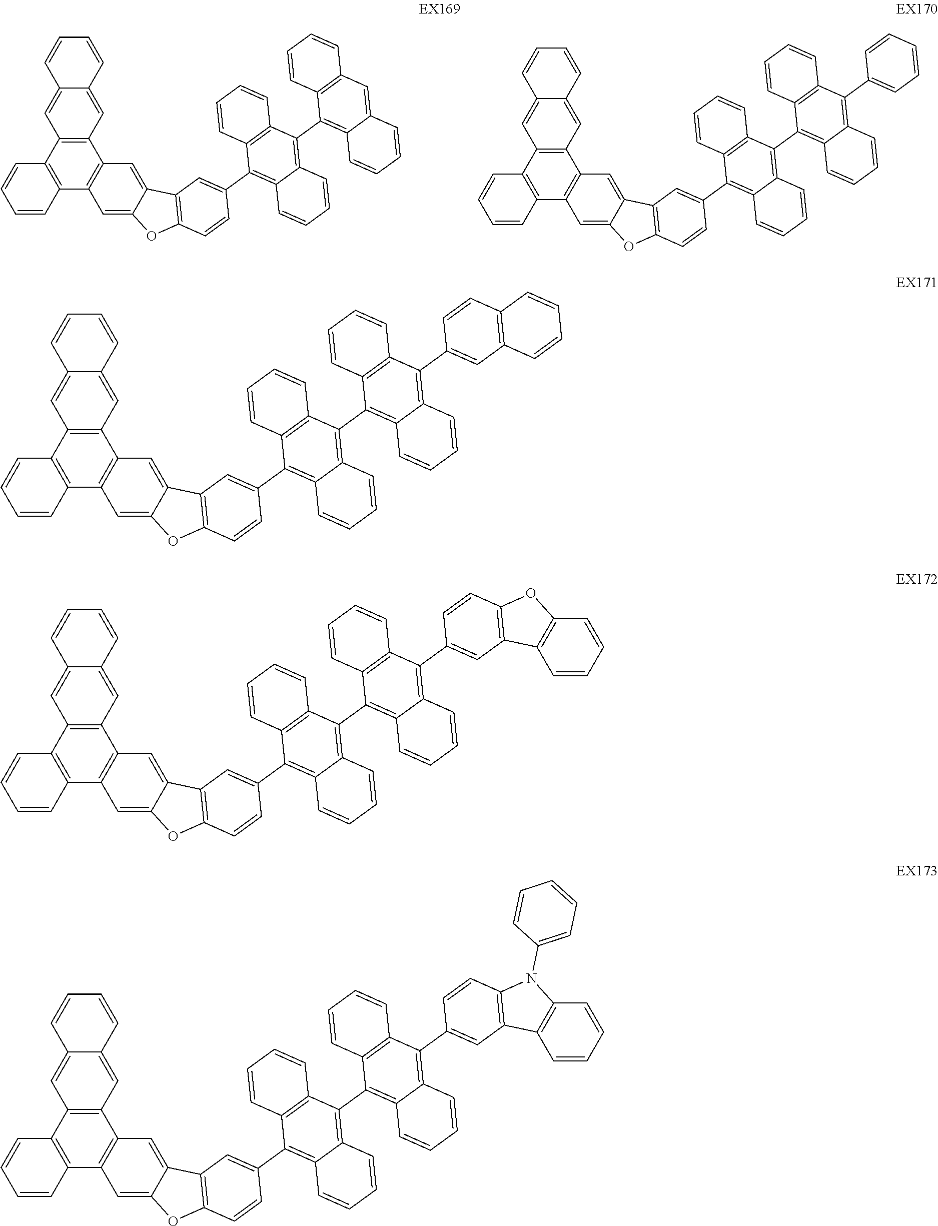

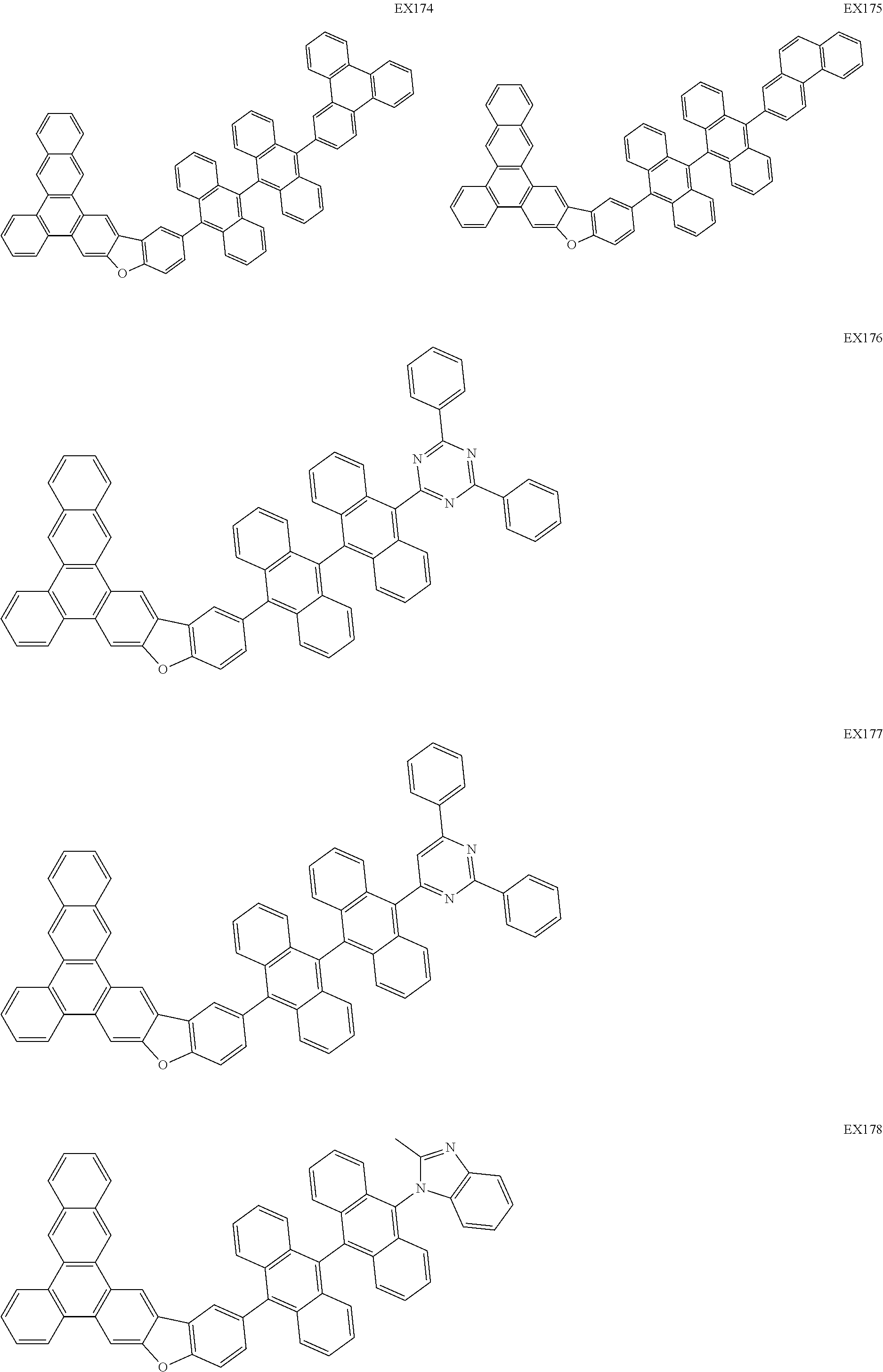

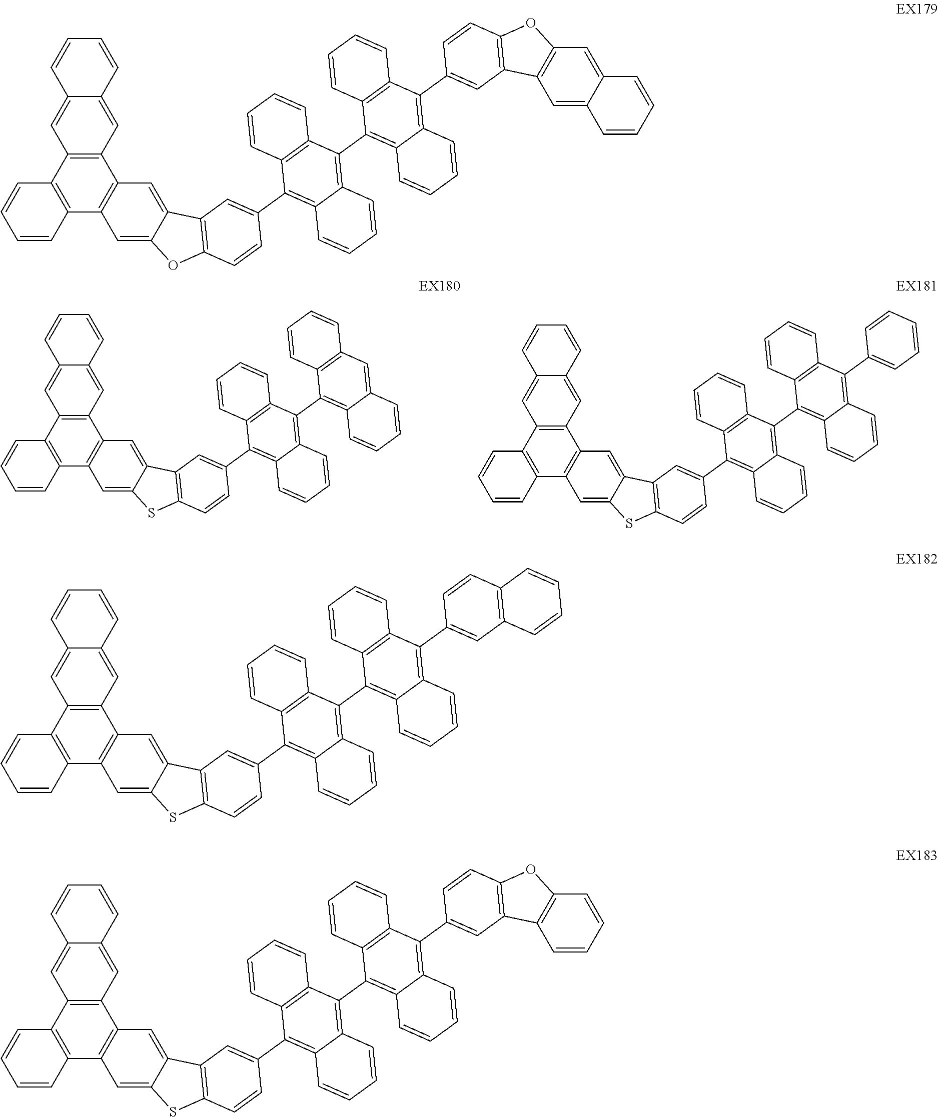

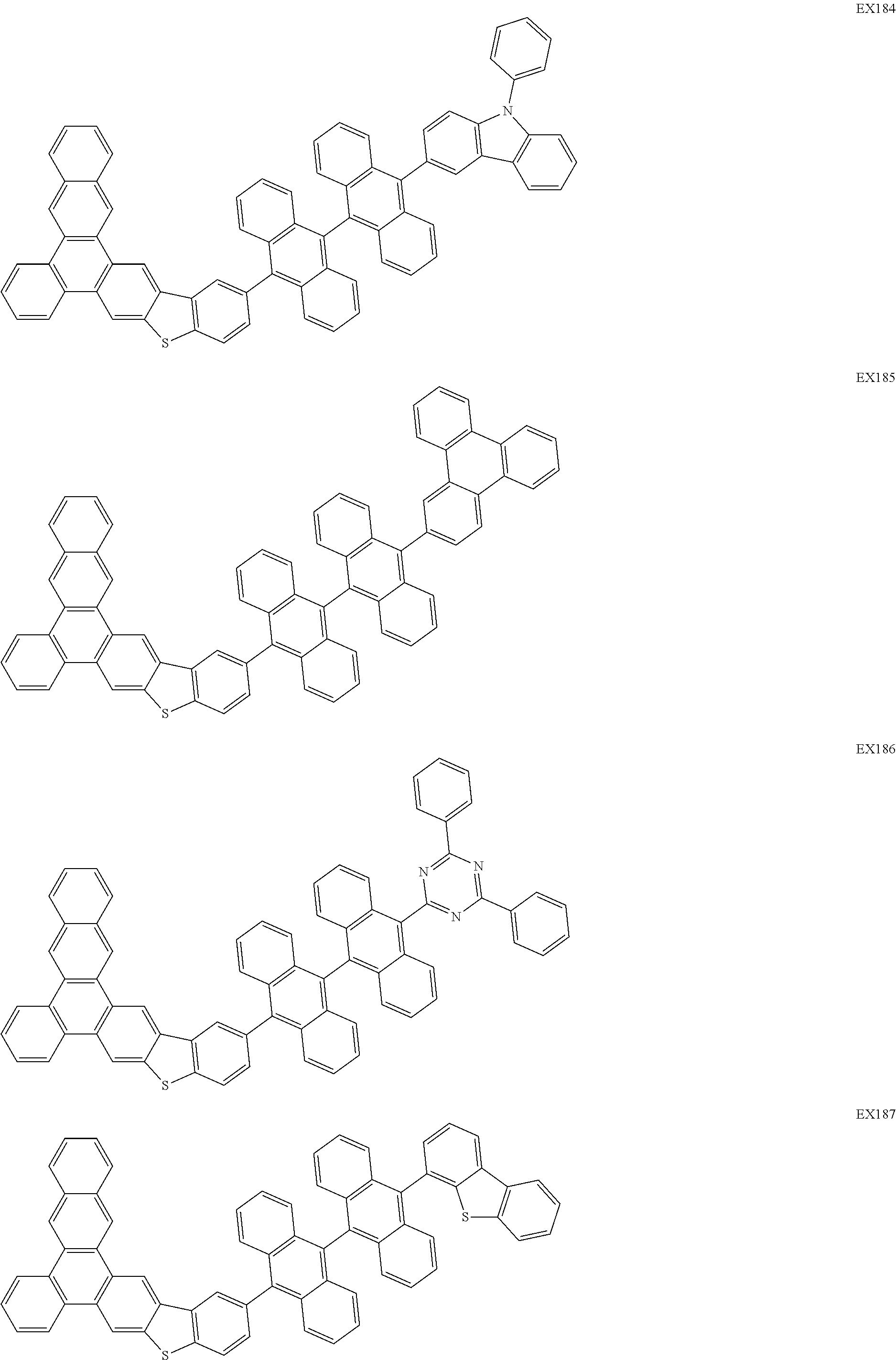

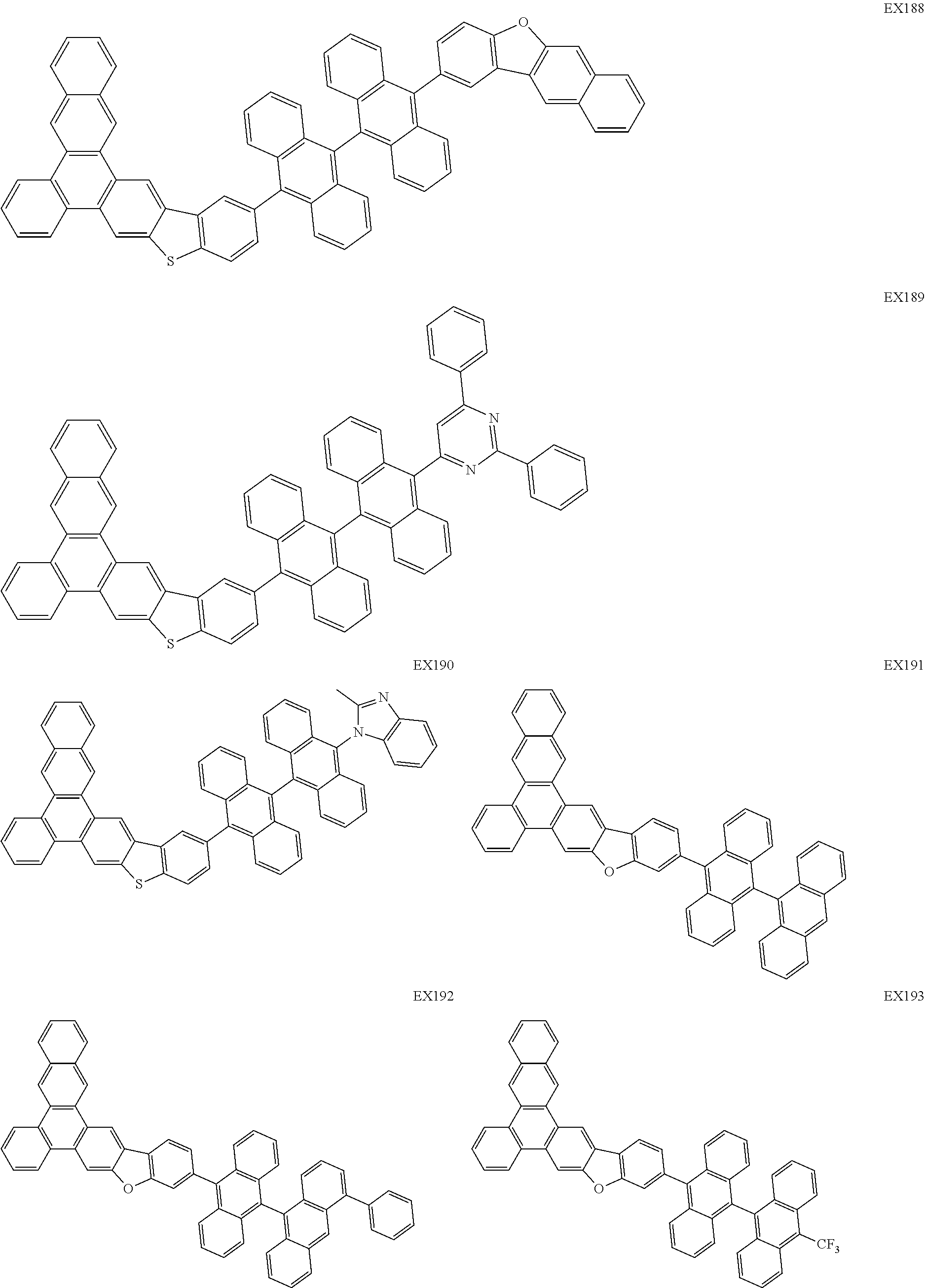

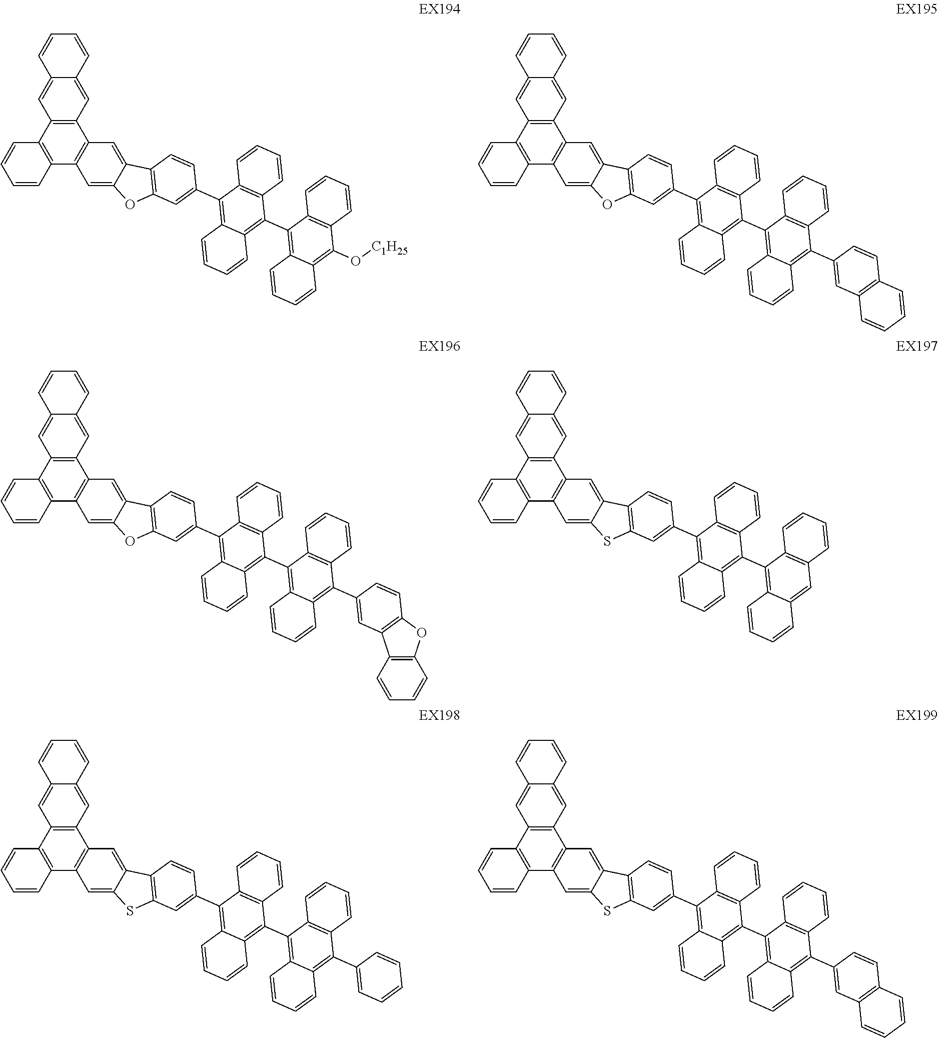

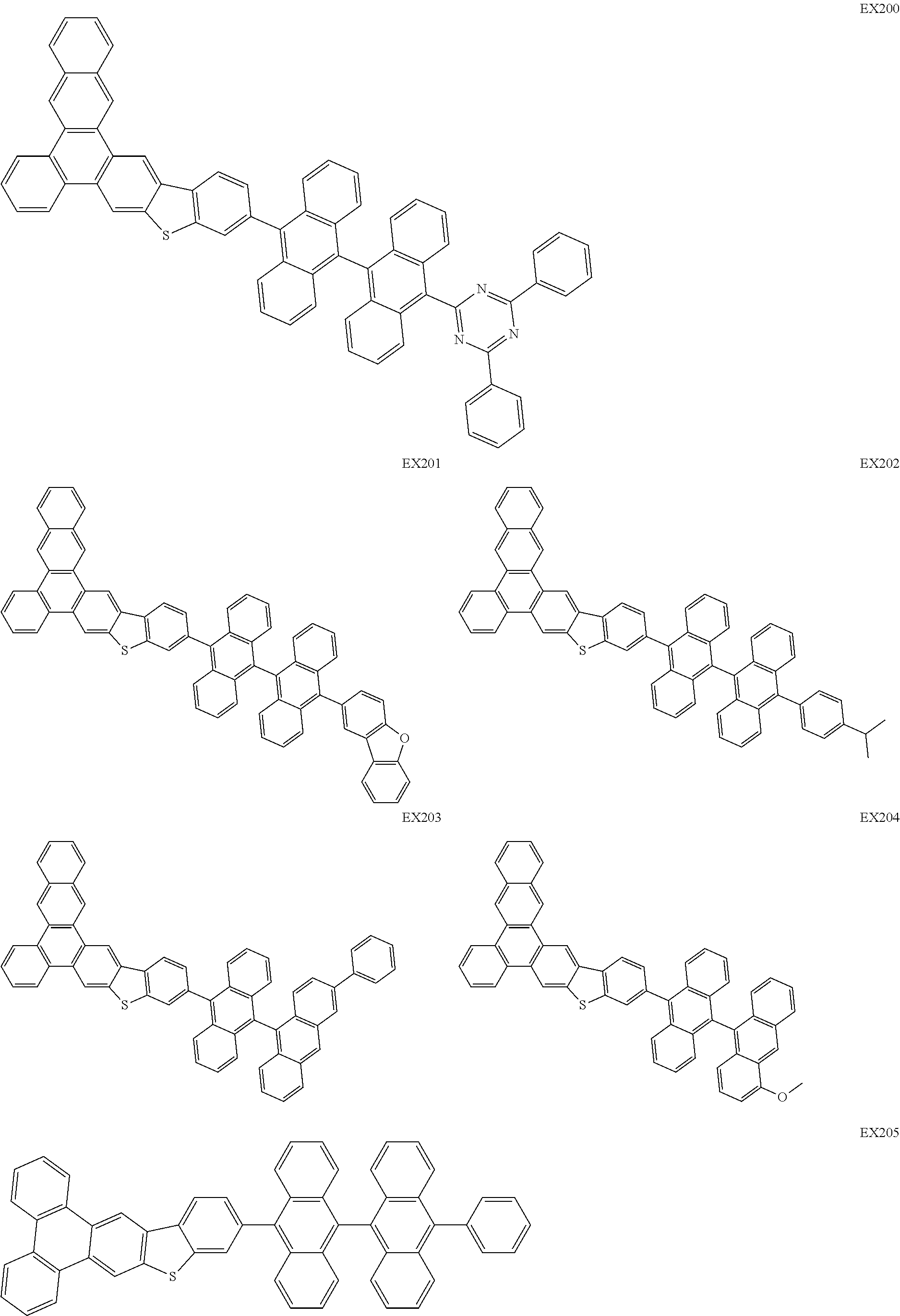

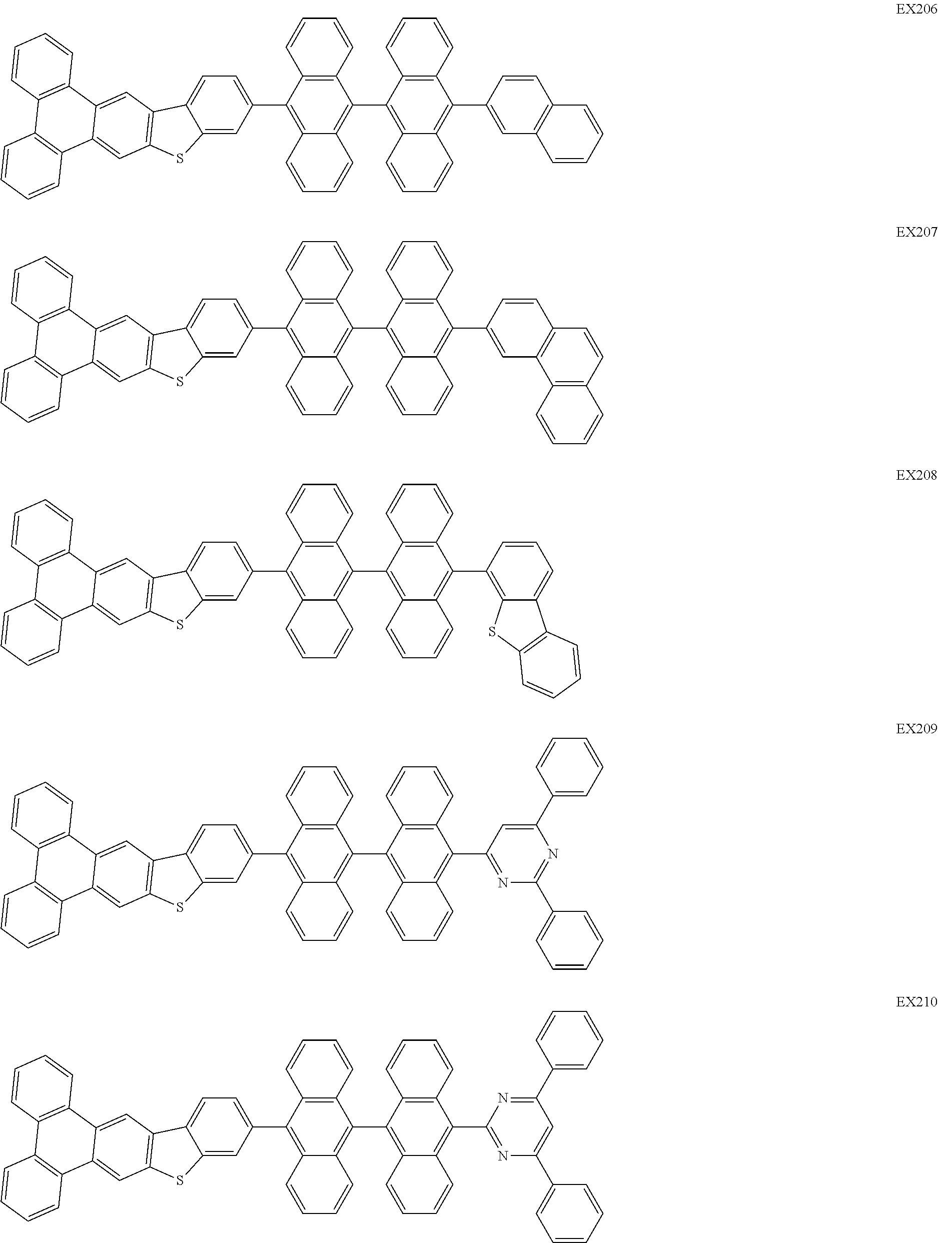

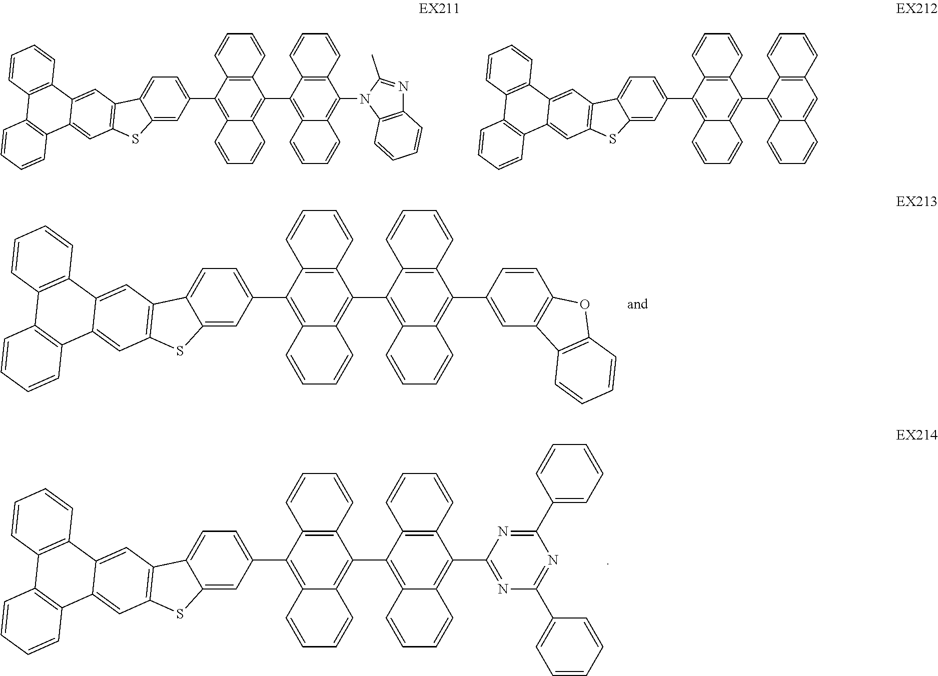

4. An organic electroluminescence device comprising a pair of electrodes having a cathode and an anode, and between the pairs of electrodes comprising at least a light emitting layer and one or more layers of organic thin film layers, wherein the light emitting layer and/or the one or more thin film layers comprise the organic compound according to claim 1.

4. The organic electroluminescence device according to claim 4, wherein the light emitting layer comprising the organic compound with a general formula (1) is a dopant material.

5. The organic electroluminescence device according to claim 4, wherein the light emitting layer comprising the organic compound with a general formula (1) is a host material.

6. The organic electroluminescence device according to claim 4, wherein the light emitting layer comprising the organic compound with a general formula (1) is a fluorescent emitter.

7. The organic electroluminescence device according to claim 4, wherein the light emitting layer emits fluorescent blue or green lights.

8. The organic electroluminescence device according to claim 4, wherein the device is an organic light emitting device.

9. The organic electroluminescent device according to claim 4, wherein the device is a lighting panel.

10. The organic electroluminescent device according to claim 4, wherein the device is a backlight panel.
